(19) United States
(12) Patent Application Publication
(10) Pub. No.: US 2026/0263512 A1
(43) Pub. Date: Sep. 10, 2026

(54) Compositions of Blood Plasma Fractions and Blood Plasma Subfractions and Their Use in Treatment of Disease

(71) Applicants: Alkahest, Inc., San Carlos, CA (US); Grifols Worldwide Operations Limited, Dublin (IE)

(72) Inventors: John G. Bromirski, Morrisville, NC (US); Rucha Deo, Sunnyvale, CA (US); Amir Hosseini, San Jose, CA (US); Viktoria Kheifets, Montain View, CA (US); Murat Kilinc, Fremont, CA (US); Annette Tennstaedt, Redwood City, CA (US)

(21) Appl. No.: 19/167,778

(22) PCT Filed: May 31, 2024
(86) PCT No.: PCT/US2024/031922

§ 371 Date: Sep. 22, 2025

Related U.S. Application Data
(60) Provisional application No. 63/470,325, filed on Jun. 1, 2023.
(60) Provisional application No. 63/556,021, filed on Feb. 21, 2024.

(51) Int. Cl. A61K 35/16; A61K 38/38; A61K 38/55; A61P 1/16; A61P 3/10; A61P 9/10; A61P 21/00; A61P 25/16; A61P 25/28; A61P 39/06

(52) U.S. Cl. CPC A61K 35/16; A61K 38/385; A61K 38/55; A61P 1/16; A61P 3/10; A61P 9/10; A61P 21/00; A61P 25/16; A61P 25/28; A61P 39/06

(57) ABSTRACT

Methods and compositions for treating and/or preventing disease are described. The compositions used in the methods include fractions and subfractions of blood plasma derived from donors, with efficacy in treating and/or preventing diseases both of the central nervous system and peripheral organs and tissues. Methods of manufacturing the compositions are also described.

### CROSS-REFERENCE TO RELATED APPLICATIONS

Pursuant to 35 U.S.C. § 119 (e), this application claims priority to the filing dates of U.S. Provisional Application Ser. No. 63/470,325 filed on Jun. 1, 2023, and U.S. Provisional Application Ser. No. 63/556,021 filed on Feb. 21, 2024; the disclosures of which applications are herein incorporated by reference.

### FIELD

This invention pertains to the prevention and treatment of disease. The invention relates to the use of blood plasma fractions and subfractions thereof to treat and/or prevent conditions often associated with aging, such as cognitive disorders, motor disorders, degenerative disorders, and inflammation.

### BACKGROUND

The following is offered as background information only and is not admitted as prior art to the present invention.

Aging is an important risk factor for multiple human diseases including cognitive impairment, neurodegeneration, cancer, arthritis, vision loss, osteoporosis, diabetes, cardiovascular disease, muscle degeneration, inflammation (including neuroinflammation) and stroke. In addition to normal synapse loss during natural aging, synapse loss is an early pathological event common to many neurodegenerative conditions and is the best correlate to the neuronal and cognitive impairment associated with these conditions. As such, aging remains the single most dominant risk factor for dementia-related neurodegenerative diseases such as Alzheimer's disease (AD) (Bishop, N. A. et al., Neural mechanisms of ageing and cognitive decline. Nature 464 (7288), 529-535 (2010); Heeden, T. et al., Insights into the ageing mind: a view from cognitive neuroscience. Nat. Rev. Neurosci. 5 (2), 87-96 (2004); Mattson, M. P., et al., Ageing and neuronal vulnerability. Nat. Rev. Neurosci. 7 (4), 278-294 (2006)). Aging affects all tissues and functions of the body including the central nervous system (CNS), and neurodegeneration and a decline in functions such as cognition or motor skills, can severely impact quality of life. Treatment for cognitive decline, motor impairment, and neurodegenerative disorders has had limited success in preventing and reversing impairment. It is therefore important to identify new treatments for maintaining cognitive integrity by protecting against, countering, or reversing the effects of aging.

Further, aging not only affects central nervous system associated disease, but diseases of the peripheral systems. These include the cardiovascular system (e.g., peripheral artery disease), musculoskeletal system (e.g., muscle degeneration and osteoporosis), and immune regulatory system (e.g., inflammation).

Blood plasma fractions have been shown previously to exhibit efficacy in reversing the effects of certain aging-associated disease and concomitant symptoms both in the CNS and peripherally (see, e.g., U.S. Patent Publication Nos. US20210128693, US20220370568, U.S. Pat. No. 20,210,145875, US20180110839, US20170340671, and US20180311280). However, the supply of said blood plasma fractions is limited to the supply of donations from plasma donors since therapeutic fractions are ultimately derived from this pool of donations. The process of blood fractionation has increased in efficiency over the decades since it was first developed in the 1940s. However, there are still inefficiencies from fractionation that produce compositions which are underutilized including blood fraction precipitates. Some even refer to these as waste products of fractionation. One such precipitate is blood plasma fraction IV-1 paste or precipitate. Thus, there is a need to identify potential therapeutic uses for such underutilized products.

### SUMMARY

The present invention is based on the production and use of blood products for treating and/or preventing disease, including those associated with aging. The present invention explores new efficiencies in the use of blood plasma fractionation. These new efficiencies include the identification of subfractions of blood plasma fraction IV-1 paste (or precipitate) that can be used to treat disease. These subfractions of blood plasma fraction IV-1 paste also provide compositions for treatment of disease.

An embodiment of the invention includes treating a subject diagnosed with a disease or disorder by administering to the subject an effective amount of a subfraction of plasma fraction IV-1 paste. Another embodiment of the invention includes administering the effective amount of a subfraction of plasma fraction IV-1 paste and subsequently monitoring the subject for improvement in symptoms related to the disease or disorder. Another embodiment of the invention includes treating a subject diagnosed with a disease or disorder by administering to the subject an effective amount of a subfraction of plasma fraction IV-1 paste wherein the subfraction of plasma fraction IV-1 paste is administered in a manner resulting in improvement of the disease or disorder, including, for example, the associated symptoms.

The current invention also recognizes that differences in protein content between different blood plasma fractions (e.g., fractions, effluents, precipitates/cryoprecipitates, pastes, Plasma Protein Fraction, Human Albumin Solution) can be responsible for preventing and/or improving certain symptoms or underlying causative factors of disease, including aging-related disease. For example, such symptoms or underlying causative factors include cognitive or motor impairments and alleviating neurodegenerative disease. Also, by way of example, and not limitation, embodiments of the current invention demonstrate that mere higher concentrations of single protein factors may not be the driving force behind improvement when treating disease. For instance, single protein factors that are concentrated in and can be derived from factor IV-1 paste (e.g., alpha-1 antitrypsin or antithrombin III) may not have the same degree or scope of therapeutic profile or effects as subfractions of factor IV-1 paste.

Blood and blood plasma from young donors have exhibited improvement and reversal of the pre-existing effects of brain aging, including at the molecular, structural, functional, and cognitive levels. (Saul A. Villeda, et al. Young blood reverses age-related impairments in cognitive function and synaptic plasticity in mice. Nature Medicine 20 659-663 (2014)). The present invention relates to fractions and effluents of the blood plasma, some of which have been traditionally used to treat patient shock, and the discovery that they are effective as methods of treatment of aging-associated cognitive impairment, reduced motor function, and neuroinflammation or neurodegenerative-related disease.

In accordance with aspects of the invention, then, methods of treatment of aging-associated cognitive impairment, age-related dementia, motor impairment, neuroinflammation, and/or neurodegenerative disease using blood product fractions of blood plasma are provided. Aspects of the methods include administering a blood plasma fraction to an individual suffering from or at risk of developing aging-associated cognitive impairment, motor impairment, neuroinflammation, or neurodegenerative disease. Additional aspects of the methods include administering a blood plasma fraction derived from a pool of donors of a specific age range to an individual suffering from or at risk of developing aging-associated cognitive impairment, motor impairment, neuroinflammation, or neurodegenerative disease. Further aspects of the methods include administration of blood plasma or Plasma Fractions using a Pulsed Dosing regimen. Also provided are reagents, devices, and kits thereof that find use in practicing the subject methods.

In an embodiment, the blood plasma fraction may be, for example, one of several blood plasma fractions obtained from a blood fractionation process, such as the Cohn fractionation process described below. In another embodiment, the blood plasma fraction may be of the type, herein referred to as “Plasma Fraction,” which is a solution comprised of normal human albumin, alpha and beta globulins, gamma globulin, and other proteins, either individually or as complexes. In another embodiment, the blood plasma fraction may be a type of blood plasma fraction known to those having skill in the art as a “Plasma Protein Fraction” (PPF). In another embodiment, the blood plasma fraction may be a “Human Albumin Solution” (HAS) fraction. In yet another embodiment, the blood plasma fraction may be one in which substantially all of the clotting factors are removed in order to retain the efficacy of the fraction with reduced risk of thromboses. Embodiments of the invention may also include administering, for example, a fraction derived from a young donor or pools of young donors. Another embodiment of the invention may include the monitoring of cognitive improvement, improved motor function, decreased neuroinflammation, or increased neurogenesis in a subject treated with a blood plasma fraction.

An embodiment of the invention includes treating a subject diagnosed with a cognitive impairment, neurodegenerative motor impairment, or a neuroinflammation-associated disease by administering to the subject an effective amount of blood plasma or Plasma Fraction. Another embodiment of the invention includes administering the effective amount of blood plasma or Plasma Fraction and subsequently monitoring the subject for improved cognitive function, improved motor function, decreased neuroinflammation, or increased neurogenesis. Another embodiment of the invention includes administering the blood plasma or Plasma Fraction via a dosing regimen of at least two consecutive days and monitoring the subject for improved cognitive function, improved motor function, decreased neuroinflammation, or increased neurogenesis at least 2 days after the date of last administration. A further embodiment of the invention includes administering the blood plasma or Plasma Fraction via a dosing regimen of at least 3, 4, 5, 6, 7, 8, 9, 10, 11, 12, 13, or 14 days and monitoring the subject for improved cognitive function, improved motor function, decreased neuroinflammation, or increased neurogenesis at least 3 days after the date of last administration. Yet another embodiment of the invention includes administering the blood plasma or Plasma Fraction via a dosing regimen of a least 2 consecutive days and after the date of last administration, monitoring for cognitive improvement, improved motor function, decreased neuroinflammation, or increased neurogenesis after the average half-life of the proteins in the blood plasma or Plasma Fraction has been reached.

An embodiment of the invention includes treating a subject diagnosed with a cognitive impairment, impaired motor function, neuroinflammation, or a decline in neurogenesis by administering to the subject an effective amount of blood plasma or Plasma Fraction, with the subject following an exercise regimen after the administration. Another embodiment of the invention includes following an exercise regimen that is prescribed to the subject. Another embodiment of the invention includes the subject exercising at a higher intensity and/or greater frequency than the subject exercised preceding the administration. Another embodiment of the invention includes the subject exercising at a similar intensity and/or frequency as the subject exercised preceding the administration.

An embodiment of the invention includes treating a subject diagnosed with a cognitive impairment, impaired motor function, neuroinflammation, or a decline in neurogenesis by administering to the subject an effective amount of blood plasma or Plasma Fraction in a subject who is undergoing, will undergo, or has received stem cell therapy. Another embodiment of the invention includes administering to a subject an effective amount of blood plasma or Plasma Fraction where the subject is undergoing, will undergo, or has received stem cell therapy, and wherein the stem cells used in the therapy can be embryonic stem cells, non-embryonic stem cells, induced pluripotent stem cells (iPSCs), cord blood stem cells, amniotic fluid stem cells, and the like. Another embodiment of the invention includes treating a subject diagnosed with traumatic spinal cord injury, stroke, retinal disease, Huntington's disease, Parkinson's Disease, Alzheimer's Disease, hearing loss, heart disease, rheumatoid arthritis, or severe burns, and who is undergoing, will undergo, or has received stem cell therapy, with an effective amount of blood plasma or Plasma Fraction.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 is a depiction of the relevant manufacturing process for fractionation of blood plasma.

FIG. 2 reports a dose-response relationship between PPF1, and IV-1 paste.

FIG. 3 is a side-by-side comparison of various plasma fractions' activities across in vitro assays.

FIG. 4 shows separation of IV-1 paste into thirteen subfractions by an anion exchange chromatography column.

FIG. 5 is a Coomassie blue staining from the 13 subfractions form IV-1 paste.

FIG. 6 reports the activity of the load (IV-1 paste), flow through (FT), and 13 subfractions in brain barrier, muscle functional, and inflammation assays.

FIG. 7 depicts the treatment paradigms for isolated primary mouse microglia.

FIG. 8 reports the phagocytotic activity of treated microglia as quantified by FACS normalized to untreated microglia as described in FIG. 7.

FIG. 9 reports the effects that subfractions of IV-1 paste have on the microglial phagocytosis assay.

FIG. 10 reports the effects that single purified protein products (A1At and ATIII) from IV-1 paste have on microglial phagocytotic activity.

FIG. 11 is a treatment paradigm for analysis of adhesion molecule surface expression in HUVEC.

FIG. 12 through FIG. 14 show the results of individual proteins and plasma fractions in the assay described in FIG. 11.

FIG. 15 through FIG. 17 report the effects of the 13 subfractions of Fraction IV-1 paste on the adhesion molecules described in FIG. 11 (VCAM1, ICAM1, and CD62E).

FIG. 18 through FIG. 21 report the effects of single protein products purified from fraction IV-1 paste (AlAT and ATIII) on adhesion molecule surface expression.

FIG. 22 is a depiction of the treatment paradigm for a Barrier Function Assay.

FIG. 23 reports the effects of Fraction IV-1 paste and PPF1 on relative TEER normalized to pre-treatment.

FIG. 24 is a dose-response study of Fraction IV-1 paste 72 hours post treatment using the TEER assay.

FIG. 25 shows the results of the TEER assay with cells that were treated with Fraction IV-1 paste, flow through (FT), and the subfractions 1 through 13 from Fraction IV-1 paste.

FIG. 26 shows the results of the TEER assay with cells that were treated with Fraction IV-1 paste, the single protein alpha-1 antitrypsin (A1AT) at various concentrations, and the single protein antithrombin III (ATIII) at various concentrations.

FIG. 27 shows a treatment paradigm for an endothelial cell proliferation assay.

FIG. 28 shows the results of the HUVEC endothelial cell proliferation assay as described in FIG. 27.

FIG. 29 shows the result of a time-response experiment for the HUVEC endothelial cell proliferation assay treated with Fraction IV-1 paste.

FIG. 30 is a depiction of the treatment paradigm for a cytokine release assay.

FIG. 31 shows the results for IL-6 release using multiple plasma fractions (HAS1, PPF1, and IV-1 paste) under TNFα stress.

FIG. 32 shows the results for IL-8 release using multiple plasma fractions (HAS1, PPF1, and IV-1 paste) or recombinant human albumin (rhAlbumin) under TNFα stress.

FIG. 33 shows the results for IL-6 release using multiple plasma fractions (HAS1, PPF1, and IV-1 paste) or recombinant human albumin (rhAlbumin) under no TNFα stress.

FIG. 34 shows the results for IL-8 release using multiple plasma fractions (HAS1, PPF1, and IV-1 paste) or recombinant human albumin (rhAlbumin) under no TNFα stress.

FIG. 35 shows the results of the thirteen Q-Sepharose subfractions from Fraction IV-1 paste tested in the cytokine release assay with TNFα stressor alongside Fraction IV-1 paste and the flow through (FT).

FIG. 36 reports release of IL-8 when testing thirteen (13) Q-Sepharose subfractions in the cytokine release assay described in FIG. 30.

FIG. 37 reports release of IL-6 when testing the single purified protein product alpha-1 antitrypsin (A1AT) derived from IV-1 paste, without TNFα stressor.

FIG. 38 reports release of IL-8 when testing the single purified protein product alpha-1 antitrypsin (A1AT) derived from IV-1 paste, without TNFα stressor.

FIG. 39 reports release of IL-6 when testing the single purified protein product alpha-1 antitrypsin (A1AT) derived from IV-1 paste, with TNFα stressor.

FIG. 40 reports release of IL-8 when testing the single purified protein product alpha-1 antitrypsin (A1AT) derived from IV-1 paste, with TNFα stressor.

FIG. 41 reports release of IL-6 when testing the single purified protein product antithrombin III (ATIII) derived from IV-1 paste, without TNFα stressor.

FIG. 42 reports release of IL-8 when testing the single purified protein product antithrombin III (ATIII) derived from IV-1 paste, without TNFα stressor.

FIG. 43 reports release of IL-6 when testing the single purified protein product antithrombin III (ATIII) derived from IV-1 paste, with TNFα stressor.

FIG. 44 reports release of IL-8 when testing the single purified protein product antithrombin III (ATIII) derived from IV-1 paste, with TNFα stressor.

FIG. 45 is a depiction of the treatment paradigm for a C2C12 myoblast differentiation assay.

FIG. 46 shows the results of the effects of the 13 subfractions of fraction IV-1 paste on the myotube formation assay described in FIG. 45.

FIG. 47 reports the effects of the single protein product alpha-1 antitrypsin (A1AT) which is purified from fraction IV-1 paste on the myotube formation assay described in FIG. 45.

FIG. 48 reports the effects of the single protein product antithrombin III (ATIII) which is purified from fraction IV-1 paste on the myotube formation assay described in FIG. 45.

FIG. 49 is a depiction of the treatment paradigm for C2C12-derirved myotube formation.

FIG. 50 shows the results of a dose-response relationship between PPF1, and fraction IV-1 paste in the glucose utilization assay described in FIG. 49.

FIG. 51 reports the results of the metabolic assay described in FIG. 49 using the 13 subfractions from fraction IV-1 paste.

FIG. 52 shows the results of separation of a Fraction IV-1 suspension into distinct protein pools by Q Sepharose chromatography.

FIG. 53 shows the elution pools corresponding to the chromatography of FIG. 52.

FIG. 54 shows gel electrophoresis results corresponding to the chromatography of FIG. 52.

FIG. 55 illustrates a treatment paradigm survival assay as described in the Experimental section below.

FIG. 56 provides the results of the treatment paradigm survival assay illustrated in FIG. 55, as described in the Experimental section below.

FIG. 57 illustrates a treatment paradigm ROS assay as described in the Experimental section below.

FIG. 58 provides the results of the treatment paradigm ROS assay illustrated in FIG. 57, as described in the Experimental section below.

FIG. 59 shows the results of the survival assay performed on dopaminergic neurons, under neurotoxin stress (MPP+1 mM), using single purified proteins (A1AT and ATIII). A1AT and ATIII were administered as described in FIG. 55.

FIG. 60 shows the results of the ROS production assay on dopaminergic neurons, under hyperoxide stress (TBHP 50 μM), single purified proteins (A1AT and ATIII). A1AT and ATIII were administered as described in FIG. 57.

FIG. 61 shows the heatmap for abundant proteins in fraction IV-1 paste, normalized to amount of protein in fraction PPF1.

FIG. 62 shows the heatmap for IGF1, IGF2, IGFBP3 and IGFALS proteins in fraction IV-1 paste, normalized to amount of protein in fraction PPF1.

### DETAILED DESCRIPTION

### 1. Introduction

The present invention relates to the identification and discovery of methods and compositions for the treatment and/or prevention of disease or bodily disorders. Described herein are methods and compositions for the treatment of subjects suffering from such diseases and disorders, which are aspects of the present invention. Also described herein are dosing regimens which improve the efficacy of the compositions to treat said diseases/disorders. An implementation of the invention includes using blood plasma fractions as treatment, such as one or more fractions or effluents obtained from blood fractionation processes, e.g., like the Cohn fractionation process described below. An embodiment of the invention includes using Plasma Fraction (a solution comprised of normal human albumin, alpha and beta globulins, gamma globulin, and other proteins either individually or as complexes, hereinafter referred to as “Plasma Fraction”). Another embodiment of the invention includes using Plasma Protein Fraction (PPF) as treatment. Another embodiment of the invention includes using Human Albumin Solution (HAS) fraction as treatment. Yet another embodiment includes using effluents from blood fractionation processes such as Effluent I or Effluent II/III described below. An additional embodiment includes the use of Fraction IV-1 paste compositions such as redissolved Fraction IV-1 paste to treat said diseases/disorders. A further embodiment includes the use of subfractions of redissolved Fraction IV-1 paste to treat the diseases/disorders. An additional embodiment includes the use of one or more thirteen (13) subfractions described herein to treat the diseases/disorders. Another embodiment of the invention includes the compositions comprising subfractions of Fraction IV-1 paste, including, but not limited to the thirteen subfractions described herein. A further embodiment includes compositions resulting from the process of fractionating Fraction IV-1 paste, including the thirteen subfractions described herein, with the process exemplified as described in Example 10.

Before describing the present invention in detail, it is to be understood that this invention is not limited to a particular method or composition described, as such may, of course, vary. It is also understood that the terminology used herein is for the purpose of describing particular embodiments only, and is not intended to be limiting, since the scope of the present invention will be limited only by the appended claims.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

It is noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein have discrete components and features which may be readily separated from or combined with the features of any of the other several embodiments without departing from the scope or the spirit of the present invention. Any recited method can be carried out in the order of events recited or in any other order which is logically possible.

### 2. Definitions

Unless otherwise defined, all technical and scientific terms used herein have the same meaning as commonly understood by one having ordinary skill in the art to which the invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those having skill in the art, and so forth.

In describing methods of the present invention, the terms “host”, “subject”, “individual” and “patient” are used interchangeably and refer to any mammal in need of such treatment according to the disclosed methods. Such mammals include, e.g., humans, ovines, bovines, equines, porcines, canines, felines, non-human primate, mice, and rats. In certain embodiments, the subject is a non-human mammal. In some embodiments, the subject is a farm animal. In other embodiments, the subject is a pet. In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). As such, subjects of the invention, include but are not limited to mammals, e.g., humans and other primates, such as chimpanzees and other apes and monkey species; and the like, where in certain embodiments the subject are humans. The term subject is also meant to include a person or organism of any age, weight or other physical characteristic, where the subjects may be an adult, a child, an infant or a newborn.

By “an individual suffering from or at risk of suffering from an aging-associated cognitive impairment” is meant an individual that is about more than 50% through its expected lifespan, such as more than 60%, e.g., more than 70%, such as more than 75%, 80%, 85%, 90%, 95% or even 99% through its expected lifespan. The age of the individual will depend on the species in question. Thus, this percentage is based on the predicted life-expectancy of the species in question. For example, in humans, such an individual is 50 year old or older, e.g., 60 years old or older, 70 years old or older, 80 years old or older, 90 years old or older, and usually no older than 100 years old, such as 90 years old., i.e., between the ages of about 50 and 100, e.g., 50 . . . 55 . . . 60 . . . 65 . . . 70 . . . 75 . . . 80 . . . 85 . . . 90 . . . 95 . . . 100 years old or older, or any age between 50-1000, that suffers from an aging-associated condition as further described below, e.g., cognitive impairment associated with the natural aging process; an individual that is about 50 years old or older, e.g., 60 years old or older, 70 years old or older, 80 years old or older, 90 years old or older, and usually no older than 100 years old, i.e., between the ages of about 50 and 100, e.g., 50 . . . 55 . . . 60 . . . 65 . . . 70 . . . 75 . . . 80 . . . 85 . . . 90 . . . 95 . . . 100 years old, that has not yet begun to show symptoms of an aging-associated condition e.g., cognitive impairment; an individual of any age that is suffering from a cognitive impairment due to an aging-associated disease, as described further below, and an individual of any age that has been diagnosed with an aging-associated disease that is typically accompanied by cognitive impairment, where the individual has not yet begun to show symptoms of cognitive impairment. The corresponding ages for non-human subjects are known and are intended to apply herein.

As used herein, “treatment” refers to any of (i) the prevention of the disease or disorder, or (ii) the reduction or elimination of symptoms of the disease or disorder. Treatment may be effected prophylactically (prior to the onset of disease) or therapeutically (following the onset of the disease). The effect may be prophylactic in terms of completely or partially preventing a disease or symptom thereof and/or may be therapeutic in terms of a partial or complete cure for a disease and/or adverse effect attributable to the disease. Thus, the term “treatment” as used herein covers any treatment of an aging-related disease or disorder in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, rejuvenation of tissue or organs, ctc. The therapeutic agent may be administered before, during or after the onset of disease. The treatment of ongoing disease, where the treatment stabilizes or reduces the undesirable clinical symptoms of the patient, is of particular interest. Such treatment may be performed prior to complete loss of function in the affected tissues. The subject therapy may be administered during the symptomatic stage of the disease, and in some cases after the symptomatic stage of the disease.

In some embodiments, the condition that is treated is an impairment in cognitive ability in an individual. By cognitive ability, or “cognition,” it is meant the mental processes that include attention and concentration, learning complex tasks and concepts, memory (acquiring, retaining, and retrieving new information in the short and/or long term), information processing (dealing with information gathered by the five senses), visuospatial function (visual perception, depth perception, using mental imagery, copying drawings, constructing objects or shapes), producing and understanding language, verbal fluency (word-finding), solving problems, making decisions, and executive functions (planning and prioritizing). By “cognitive decline”, it is meant a progressive decrease in one or more of these abilities, e.g., a decline in memory, language, thinking, judgment, etc. By “an impairment in cognitive ability” and “cognitive impairment”, it is meant a reduction in cognitive ability relative to a healthy individual, e.g., an age-matched healthy individual, or relative to the ability of the individual at an earlier point in time, e.g., 2 weeks, 1 month, 2 months, 3 months, 6 months, 1 year, 2 years, 5 years, or 10 years or more previously. By “aging-associated cognitive impairment,” it is meant an impairment in cognitive ability that is typically associated with aging, including, for example, cognitive impairment associated with the natural aging process, e.g., mild cognitive impairment (M.C.I.); and cognitive impairment associated with an aging-associated disorder, that is, a disorder that is seen with increasing frequency with increasing senescence, e.g., a neurodegenerative condition such as Alzheimer's disease, Parkinson's disease, frontotemporal dementia, Huntington disease, amyotrophic lateral sclerosis, multiple sclerosis, glaucoma, myotonic dystrophy, vascular dementia, and the like.

In some embodiments, the condition that is treated is an aging-associated impairment in motor ability in an individual. By motor ability, it is meant the motor processes that include the ability to perform complex muscle-and-nerve actions that produce movement such as fine motor skills producing small or precise movements (e.g., writing, tying shoes) and gross motor skills for large movements (e.g., walking, running, kicking). By “motor decline”, it is meant a progressive decrease in one or more of these abilities, e.g., a decline in find movement or gross motor skills, etc. By “motor impaired” and “motor impairment”, it is meant a reduction in motor ability/skills relative to a healthy individual, e.g., an age-matched healthy individual, or relative to the ability of the individual at an earlier point in time, e.g., 2 weeks, 1 month, 2 months, 3 months, 6 months, 1 year, 2 years, 5 years, or 10 years or more previously. By “aging-associated motor impairment,” it is meant an impairment or decline in motor ability that is typically associated with aging, including, for example, motor impairment associated with the natural aging process and motor impairment or decline associated with an aging-associated disorder, that is, a disorder that is seen with increasing frequency with increasing senescence, e.g., a neurodegenerative condition such as Parkinson's disease, amyotrophic lateral sclerosis, and the like.

In some embodiments, the condition that is treated is an increase in neuroinflammation in an individual. By “neuroinflammation” it is meant biochemical and cellular responses of the nervous system to injury, infection, or neurodegenerative diseases. Such responses are directed at decreasing the triggering factors by involving central nervous system immunity to defend against potential harm. Neurodegeneration occurs in the central nervous system and exhibits hallmarks of loss of neuronal structure and function. Neuroinflammatory diseases or neuroinflammatory-associated conditions or diseases, includes by way of example and not limitation, neurodegenerative diseases such as Alzheimer's disease; Parkinson's disease, multiple sclerosis and the like.

Blood Products Comprising Plasma Components. In practicing the subject methods, a blood product comprising plasma components is administered to an individual in need thereof, e.g., an individual suffering or at risk of suffering from a cognitive or motor impairment, neuroinflammation and/or age-related dementia. As such, methods according to embodiments of the invention include administering a blood product comprising plasma components from an individual (the “donor individual”, or “donor”) to an individual at least at risk of suffering or suffering from cognitive or motor impairment, neuroinflammation, neurodegeneration, and/or age-related dementia (the “recipient individual” or “recipient”). By a “blood product comprising plasma components,” it is meant any product derived from blood that comprises plasma (e.g. whole blood, blood plasma, or fractions thereof). The term “plasma” is used in its conventional sense to refer to the straw-colored/pale-yellow liquid component of blood composed of about 92% water, 7% proteins such as albumin, gamma globulin, anti-hemophilic factor, and other clotting factors, and 1% mineral salts, sugars, fats, hormones and vitamins. Non-limiting examples of plasma-comprising blood products suitable for use in the subject methods include whole blood treated with anti-coagulant (e.g., EDTA, citrate, oxalate, heparin, etc.), blood products produced by filtering whole blood to remove white blood cells (“lcukorcduction”), blood products consisting of plasmapheretically-derived or apheretically-derived plasma, fresh-frozen plasma, blood products consisting essentially of purified plasma, and blood products consisting essentially of plasma fractions. In some instances, plasma product that is employed is a non-whole blood plasma product, by which is meant that the product is not whole blood, such that it lacks one or more components found in whole blood, such as erythrocytes, leukocytes, etc., at least to the extent that these components are present in whole blood. In some instances, the plasma product is substantially, if not completely, acellular, where in such instances the cellular content may be 5% by volume or less, such as 1% or less, including 0.5% or less, where in some instances acellular plasma fractions are those compositions that completely lack cells, i.e., they include no cells.

Collection of blood products comprising plasma components. Embodiments of the methods described herein include administration of blood products comprising plasma components which can be derived from donors, including human volunteers. The term, “human-derived” can refer to such products. Methods of collection of plasma comprising blood products from donors are well-known in the art. (See, e.g., AABB TECHNICAL MANUAL, (Mark A. Fung, et al., eds., 18th ed. 2014), herein incorporated by reference).

In one embodiment, donations are obtained by venipuncture. In another embodiment, the venipuncture is only a single venipuncture. In another embodiment, no saline volume replacement is employed. In a preferred embodiment, the process of plasmapheresis is used to obtain the plasma comprising blood products. Plasmapheresis can comprise the removal of a weight-adjusted volume of plasma with the return of cellular components to the donor. In the preferred embodiment, sodium citrate is used during plasmapheresis in order to prevent cell clotting. The volume of plasma collected from a donor is preferably between 690 to 880 mL after citrate administration, and preferably coordinates with the donor's weight.

### 3. Plasma Fractions

During the Second World War, there arose a need for a stable plasma expander which could be employed in the battlefield when soldiers lost large amounts of blood. As a result, methods of preparing freeze-dried plasma were developed. However, use of freeze-dried plasma was difficult in combat situations since reconstitution required sterile water. As an alternative, Dr. E. J. Cohn suggested that albumin could be used, and prepared a ready-to-use stable solution that could be introduced immediately for treatment of shock. (See Johan, Current Approaches to the Preparation of Plasma Fractions in (Biotechnology of Blood) 165 (Jack Goldstein ed., 1st ed. 1991)). Dr. Cohn's procedure of purifying plasma fractions utilized cold ethanol for its denaturing effect and employs changes in pH and temperature to achieve separation.

An embodiment of the methods described herein includes the administration of plasma fractions to a subject. Fractionation is the process by which certain protein subsets are separated from plasma. Fractionation technology is known in the art and relies on steps developed by Cohn et al. during the 1940s. (E. Cohn, Preparation and properties of serum and plasma proteins. IV. A system for the separation into fractions of the protein and lipoprotein components of biological tissues and fluids. 68 J Am Chem Soc 459 (1946), herein incorporated by reference). Several steps are involved in this process, each step involving specific ethanol concentrations as well as pH, temperature, and osmolality shifts which result in selective protein precipitation. Precipitates are also separated via centrifugation or precipitation. The original “Cohn fractionation process” involved separation of proteins through precipitates into five fractions, designated fraction I, fraction II+III, fraction IV-1, fraction IV-4 and fraction V. Albumin was the originally identified endpoint (fraction V) product of this process. In accordance with embodiments of the invention, each fraction (or effluent from a prior separation step) contains or potentially contains therapeutically-useful protein fractions. (See Thierry Burnouf, Modern Plasma Fractionation, 21 (2) Transfusion Medicine Reviews 101 (2007); Adil Denizli, Plasma fractionation: conventional and chromatographic methods for albumin purification, 4 J. Biol. & Chem. 315, (2011); and T. Brodniewicz-Proba, Human Plasma Fractionation and the Impact of New Technologies on the Use and Quality of Plasma-derived Products, 5 Blood Reviews 245 (1991), and U.S. Pat. Nos. 3,869,431, 5,110,907, 5,219,995, 7,531,513, and 8,772,461 which are herein incorporated by reference). Adjustment of the above experimental parameters can be made in order to obtain specific protein fractions.

More recently, fractionation has reached further complexity, and as such, comprises additional embodiments of the invention. This recent increase in complexity has occurred through: the introduction of chromatography resulting in isolation of new proteins from existing fractions like cryoprecipitate or paste, cryo-poor plasma, and Cohn fractions; increasing IgG recovery by integrating chromatography and the ethanol fractionation process; and viral reduction/inactivation/removal. (Id.) In order to capture proteins at physiological pH and ionic strength, anion-exchange chromatography can be utilized (e.g., use of Q-Sepharose columns). This preserves functional activity of proteins and/or protein fractions. Heparin and monoclonal antibodies are also used in affinity chromatography. One of ordinary skill in the art would recognize that the parameters described above may be adjusted to obtain specifically-desired plasma protein-containing fractions.

In an embodiment of the invention, blood plasma is fractionated in an industrial setting. Frozen plasma is thawed at 1° C. to 4° C. Continuous refrigerated centrifugation is applied to the thawed plasma and cryoprecipitate isolated. Recovered cryoprecipitate is frozen at −30° C. or lower and stored. The cryoprecipitate-poor (“cryo-poor”) plasma is immediately processed for capture (via, for example, primary chromatography) of labile coagulation factors such as factor IX complex and its components as well as protease inhibitors such as antithrombin and C1 esterase inhibitor. Serial centrifugation and precipitate isolation can be applied in subsequent steps. Such techniques are known to one of ordinary skill in the art and are described, for example, in U.S. Pat. Nos. 4,624,780, 5,219,995, 5,288,853, and U.S. patents application Nos. 20140343255 and 20150343025, which disclosures are incorporated by reference in their entirety herein.

In an embodiment of the invention, the plasma fraction may comprise a plasma fraction containing a substantial concentration of albumin. In another embodiment of the invention, the plasma fraction may comprise a plasma fraction containing a substantial concentration of IgG or intravenous immune globulin (IGIV) (e.g., Gamunex-C®). In another embodiment of the invention the plasma fraction may comprise an IGIV plasma fraction, such as Gamunex-C® which has been substantially depleted of immune globulin (IgG) by methods well-known by one of ordinary skill in the art, such as for example, Protein A-mediated depletion. (See Keshishian, H., et al., Multiplexed, Quantitative Workflow for Sensitive Biomarker Discovery in Plasma Yields Novel Candidates for Early Myocardial Injury, Molecular & Cellular Proteomics, 14 at 2375-93 (2015)). In an additional embodiment, the blood plasma fraction may be one in which substantially all the clotting factors are removed in order to retain the efficacy of the fraction with reduced risk of thromboses. For example, the plasma fraction may be a plasma fraction as described in U.S. Patent No. 62/376,529 filed on Aug. 18, 2016; the disclosure of which is incorporated by reference in its entirety herein.

### 4. Albumin Products

To those having ordinary skill in the art, there are two general categories of Albumin Plasma Products (“APP”): plasma protein fraction (“PPF”) and human albumin solution (“HAS”). PPF is derived from a process with a higher yield than HAS but has a lower minimum albumin purity than HAS (>83% for PPF and >95% for HAS). (Production of human albumin solution: a continually developing colloid, P. Matejtschuk et al., British J. of Anaesthesia 85 (6): 887-95, at 888 (2000)). In some instances, PPF has albumin purity of between 83% and 95% or alternatively 83% and 96%. The albumin purity can be determined by electrophoresis or other quantifying assays such as, for example, by mass spectrometry. Additionally, some have noted that PPF has a disadvantage because of the presence of protein “contaminants” such as PKA. Id. As a consequence, PPF preparations have lost popularity as Albumin Plasma Products, and have even been delisted from certain countries' Pharmacopoeias. Id. Contrary to these concerns, the invention makes beneficial use of these “contaminants.” Besides α, β, and γ globulins, as well as the aforementioned PKA, the methods of the invention utilize additional proteins or other factors within the “contaminants” that promote processes such as neurogenesis, neuronal cell survival, improved cognition or motor function and decreased neuroinflammation.

Those of skill in the art will recognize that there are, or have been, several commercial sources of PPF (the “Commercial PPF Preparations.”) These include Plasma-Plex™ PPF (Armour Pharmaceutical Co., Tarrytown, NY), Plasmanate™ PPF (Grifols, Clayton, NC), Plasmatein™ (Alpha Therapeutics, Los Angeles, CA), and Protenate™ PPF (Baxter Labs, Inc. Deerfield, IL).

Those of skill in the art will also recognize that there are, or have been, several commercial sources of HAS (the “Commercial HAS Preparations.”) These include Albuminar™ (CSL Behring), AlbuRx™ (CSL Behring), Albutein™ (Grifols, Clayton, NC), Buminate™ (Baxatla, Inc., Bannockburn, IL), Flexbumin™ (Baxatla, Inc., Bannockburn, IL), and Plasbumin™ (Grifols, Clayton, NC).

a. Plasma Protein Fraction (Human) (PPF)

According to the United States Food and Drug Administration (“FDA”), “Plasma Protein Fraction (Human),” or PPF, is the proper name of the product defined as “a sterile solution of protein composed of albumin and globulin, derived from human plasma.” (Code of Federal Regulations “CFR” 21 CFR 640.90 which is herein incorporated by reference). PPF's source material is plasma recovered from Whole Blood prepared as prescribed in 21 CFR 640.1-640.5 (incorporated by reference herein), or Source Plasma prepared as prescribed in 21 CFR 640.60-640.76 (incorporated by reference herein).

PPF is tested to determine it meets the following standards as per 21 CFR 640.92 (incorporated by reference herein):
(a) The final product shall be a 5.0+/−0.30 percent solution of protein; and
(b) The total protein in the final product shall consist of at least 83 percent albumin, and no more than 17 percent globulins. No more than 1 percent of the total protein shall be gamma globulin. The protein composition is determined by a method that has been approved for each manufacturer by the Director, Center for Biologics Evaluation and Research, Food and Drug Administration.

As used herein, “Plasma Protein Fraction” or “PPF” refers to a sterile solution of protein composed of albumin and globulin, derived from human plasma, with an albumin content of at least 83% with no more than 17% globulins (including α1, α2, β, and γ globulins) and other plasma proteins, and no more than 1% gamma globulin as determined by electrophoresis. (Hink, J. H., Jr., et al., Preparation and Properties of a Heat-Treated Human Plasma Protein Fraction, VOX SANGUINIS 2 (174) (1957)). PPF can also refer to a solid form, which when suspended in solvent, has similar composition. The total globulin fraction can be determined through subtracting the albumin from the total protein. (Busher, J., Serum Albumin and Globulin, CLINICAL METHODS: THE HISTORY, PHYSICAL, AND LABORATORY EXAMINATIONS, Chapter 10, Walker H K, Hall W D, Hurst J D, eds. (1990)).

b. Albumin (Human) (HAS)

According to the FDA, “Albumin (Human)” (also referred to herein as “HAS”) is the proper name of the product defined as “sterile solution of the albumin derived from human plasma.” (Code of Federal Regulations “CFR” 21 CFR 640.80 which is herein incorporated by reference.) The source material for Albumin (Human) is plasma recovered from Whole Blood prepared as prescribed in 21 CFR 640.1-640.5 (incorporated by reference herein), or Source Plasma prepared as prescribed in 21 CFR 640.60-640.76 (incorporated by reference herein). Other requirements for Albumin (Human) are listed in 21 CFR 640.80-640.84 (incorporated by reference herein).

Albumin (Human) is tested to determine if it meets the following standards as per 21 CFR 640.82:
(a) Protein concentration. Final product shall conform to one of the following concentrations: 4.0+/−0.25 percent; 5.0+/−0.30 percent; 20.0+/−1.2 percent; and 25.0+/−1.5 percent solution of protein.
(b) Protein composition. At least 96 percent of the total protein in the final product shall be albumin, as determined by a method that has been approved for each manufacturer by the Director, Center for Biologics Evaluation and Research, Food and Drug Administration.

As used herein, “Albumin (Human)” or “HAS” refers to a to a sterile solution of protein composed of albumin and globulin, derived from human plasma, with an albumin content of at least 95%, with no more than 5% globulins (including α1, α2, β, and γ globulins) and other plasma proteins. HAS can also refer to a solid form, which when suspended in solvent, has similar composition. The total globulin fraction can be determined through subtracting the albumin from the total protein.

As can be recognized by one having ordinary skill in the art, PPF and HAS fractions can also be freeze-dried or in other solid form. Such preparations, with appropriate additives, can be used to make tablets, powders, granules, or capsules, for example. The solid form can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

### 5. Clotting Factor-Reduced Fractions

Another embodiment of the invention uses a blood plasma fraction from which substantially all of the clotting factors are removed in order to retain the efficacy of the fraction with reduced risk of thromboses. Conveniently, the blood product can be derived from a young donor or pool of young donors and can be rendered devoid of IgM in order to provide a young blood product that is ABO compatible. Currently, plasma that is transfused is matched for ABO blood type, as the presence of naturally occurring antibodies to the A and B antigens can result in transfusion reactions. IgM appears to be responsible for transfusion reactions when patients are given plasma that is not ABO matched. Removal of IgM from blood products or fractions helps eliminate transfusion reactions in subjects who are administered the blood products and blood plasma fractions of the invention.

Accordingly, in one embodiment, the invention is directed to a method of treating or preventing an aging-related condition such as cognitive or motor impairment, neuroinflammation or neurodegeneration in a subject. The method comprises: administering to the subject a blood product or blood fraction derived from whole-blood from an individual or pool of individuals, wherein the blood product or blood fraction is substantially devoid of (a) at least one clotting factor and/or (b) IgM. In some embodiments, the individual(s) from whom the blood product or blood fraction is derived are young individuals. In some embodiments, the blood product is substantially devoid of at least one clotting factor and IgM. In certain embodiments, the blood product is substantially devoid of fibrinogen (Factor I). In additional embodiments, the blood product substantially lacks erythrocytes and/or leukocytes. In further embodiments, the blood product is substantially acellular. In other embodiments, the blood product is derived from plasma. Such embodiments of the invention are further supported by U.S. Patent Application No. 62/376,529 filed on Aug. 18, 2016, which is incorporated by reference in its entirety herein.

### 6. Protein-Enriched Plasma Protein Products Treatment

Additional embodiments of the invention use plasma fractions with reduced albumin concentration compared to PPF, but with increased amounts of globulins and other plasma proteins (what have been referred to by some as “contaminants”). The embodiments, as with PPF, HAS, Effluent I, and Effluent II/III are all effectively devoid of clotting factors. Such plasma fractions are hereinafter referred to as “protein-enriched plasma protein products”. For example, an embodiment of the invention may use a protein-enriched plasma protein product comprised of 82% albumin and 18% α, β, and γ globulins and other plasma proteins. Another embodiment of the invention may use a protein-enriched plasma protein product comprised of 81% albumin and 19% of α, β, and γ globulins and/or other plasma proteins. Another embodiment of the invention may use a protein-enriched plasma protein product comprised of 80% albumin and 20% of α, β, and γ globulins and/or other plasma proteins. Additional embodiments of the invention may use protein-enriched plasma protein products comprised of 70-79% albumin and a corresponding 21-30% of α, β, and γ globulins and other plasma proteins. Additional embodiments of the invention may use protein-enriched plasma protein products comprised of 60-69% albumin and a corresponding 31-40% of α, β, and γ globulins and other plasma proteins. Additional embodiments of the invention may use protein-enriched plasma protein products comprised of 50-59% albumin and a corresponding 41-50% of α, β, and γ globulins and other plasma proteins. Additional embodiments of the invention may use protein-enriched plasma protein products comprised of 40-49% albumin and a corresponding 51-60% of α, β, and γ globulins and other plasma proteins. Additional embodiments of the invention may use protein-enriched plasma protein products comprised of 30-39% albumin and a corresponding 61-70% of α, β, and γ globulins and other plasma proteins. Additional embodiments of the invention may use protein-enriched plasma protein products comprised of 20-29% albumin and a corresponding 71-80% of α, β, and γ globulins and other plasma proteins. Additional embodiments of the invention may use protein-enriched plasma protein products comprised of 10-19% albumin and a corresponding 81-90% of α, β, and γ globulins and other plasma proteins. Additional embodiments of the invention may use protein-enriched plasma protein products comprised of 1-9% albumin and a corresponding 91-99% of α, β, and γ globulins and other plasma proteins. A further embodiment of the invention may use protein-enriched plasma protein products comprised of 0% albumin and 100% of α, β, and γ globulins and other plasma proteins.

Embodiments of the invention described above may also have total gamma globulin concentrations of 1-5%.

The specific concentrations of proteins in a plasma fraction may be determined using techniques well-known to a person having ordinary skill in the relevant art. By way of example, and not limitation, such techniques include electrophoresis, mass spectrometry, ELISA analysis, and Western blot analysis.

### 7. Preparation of Plasma Fractions

Methods of preparing PPF and other plasma fractions are well-known to those having ordinary skill in the art. An embodiment of the invention allows for blood used in the preparation of human plasma protein fraction to be collected in flasks with citrate or anticoagulant citrate dextrose solution for inhibition of coagulation, with further separation of Fractions I, II+III, IV, and PPF as per the method disclosed in Hink et al. (See Hink, J. H., Jr., et al., Preparation and Properties of a Heat-Treated Human Plasma Protein Fraction, VOX SANGUINIS 2 (174) (1957), herein incorporated by reference.) According to this method, the mixture can be collected to 2-8° C. The plasma can then subsequently be separated by centrifugation at 7° C., removed, and stored at −20° C. The plasma can then be thawed at 37° C. and fractionated, preferably within eight hours after removal from −20° C. storage.

Plasma can be separated from Fraction I using 8% ethanol at pH 7.2 and a temperature at −2 to −2.5° C. with protein concentration of 5.1 to 5.6 percent. Cold 53.3 percent ethanol (176 mL/L of plasma) with acetate buffer (200 mL 4M sodium acetate, 230 mL glacial acetic acid quantum satis to 1 L with H₂O) can be added using jets at a rate, for example, of 450 mL/minute during the lowering the plasma temperature to −2° C. Fraction I can be separated and removed from the effluent (Effluent I) through ultracentrifugation. Fibrinogen can be obtained from Fraction I as per methods well-known to those having ordinary skill in the art.

Fraction II+III can be separated from Effluent I through adjustment of the effluent to 21 percent ethanol at pH 6.8, temperature at −6° C., with protein concentration of 4.3 percent. Cold 95 percent ethanol (176 mL/L of Effluent I) with 10 M acetic acid used for pH adjustment can be added using jets at a rate, for example, of 500 mL/minute during the lowering of the temperature of Effluent I to −6° C. The resulting precipitate (Fraction II+III) can be removed by centrifugation at −6° C. Gamma globulin can be obtained from Fraction II+III using methods well-known to those having ordinary skill in the art.

Fraction IV-1 can be separated from Effluent II+III (“Effluent II/III”) through adjustment of the effluent to 19 percent ethanol at pH 5.2, temperature at −6° C., and protein concentration of 3 percent. H₂O and 10 M acetic acid used for pH adjustment can be added using jets while maintaining Effluent II/III at −6° C. for 6 hours. Precipitated Fraction VI-1 can be settled at −6° C. for 6 hours and subsequently separated from the effluent by centrifugation at the same temperature. Stable plasma protein fraction can be recovered from Effluent IV-1 through adjustment of the ethanol concentration to 30 percent at pH 4.65, temperature −7° C. and protein concentration of 2.5 percent. This can be accomplished by adjusting the pH of Effluent IV-1 with cold acid-alcohol (two parts 2 M acetic acid and one-part 95 percent ethanol). While maintaining a temperature of −7° C., to every liter of adjusted Effluent IV-1 170 mL cold ethanol (95%) is added. Proteins that precipitate can be allowed to settle for 36 hours and subsequently removed by centrifugation at −7° C.

The recovered proteins (stable plasma protein fraction) can be dried (e.g. by freeze drying) to remove alcohol and H₂O. The resulting dried powder can be dissolved in sterile distilled water, for example using 15 liters of water/kg of powder, with the solution adjusted to pH 7.0 with 1 M NaOH. A final concentration of 5 percent protein can be achieved by adding sterile distilled water containing sodium acctyl tryptophanate, sodium caprylate, and NaCl, adjusting to final concentrations of 0.004 M acetyl tryptophanate, 0.004 M caprylate, and 0.112 M sodium. Finally, the solution can be filtered at 10° C. to obtain a clear solution and subsequently heat-treated for inactivation of pathogens at 60° C. for at least 10 hours.

Fraction IV-1 paste can be redissolved in 0.005 M Tris buffer, followed by heating and addition of 0.11 M NaCl. The suspension can then be used for subfractionation by anion exchange to preserve protein integrity and function for biological testing or treatment. The method of redissolving fraction IV-1 paste is further described in Hoffman DL, AM J Med (1989) 87 (suppl 3B): 23S-26S which is herein incorporated by reference.

Fraction IV-1 paste contains appreciable levels of at least two individual protein products. One is alpha-1 antitrypsin (abbreviated as “A1AT” or “AAT”), and also referred to as alpha-1 antiprotease. The second one is antithrombin III (“ATIII”).

Alpha-1 antitrypsin (AAT) is a protein that is primarily produced in the liver and circulates in the bloodstream. It is part of the serine protease inhibitor family (Serpins) and it regulates the activity of certain enzymes in the body. AAT can inhibit the activity of an enzyme called neutrophil elastase, which is produced by white blood cells and can damage tissues if not properly controlled. AAT deficiency can lead to a condition called alpha-1 antitrypsin deficiency. This is a genetic disorder that can cause lung and liver disease. This is due to a lack of AAT allowing neutrophil elastase to damage lung tissue, leading to emphysema, and liver tissue, leading to cirrhosis. Testing for AAT levels in the blood can help diagnose alpha-1 antitrypsin deficiency.

Antithrombin III is a proteinase inhibitor, also related to serpins. It acts as a regulator of hemostasis and thrombosis. It is useful in treating congenital ATIII deficiency primarily by regulating blood coagulation through inhibition of thrombin.

Subfractionation can be performed by multiple techniques including chromatography utilizing an anion-exchange column such as a Q-Sepharose Fast Flow column. Fractionation to create specific subfractions described herein is further elucidated in EXAMPLE 10.

One having ordinary skill in the art would recognize that each of the different fractions and effluents described above could be used with the methods of the invention to treat disease. For example, and not by way of limitation, Effluents I or Effluent II/III may be utilized to treat such diseases as cognitive, motor, and neurodegenerative disorders and are embodiments of the invention.

The preceding methods of preparing plasma fractions and plasma protein fraction (PPF) are only exemplary and involves merely embodiments of the invention. One having ordinary skill in the art would recognize that these methods can vary. For example, pH, temperature, and ethanol concentration, among other things can be adjusted to produce different variations of plasma fractions and plasma protein fraction in the different embodiments and methods of the invention. In another example, additional embodiments of the invention contemplate the use of nanofiltration for the removal/inactivation of pathogens from plasma fractions and plasma protein fraction.

An additional embodiment of the invention contemplates methods and composition using and/or comprising additional plasma fractions. For example, the invention, among other things, demonstrates that specific concentrations of albumin are not critical for improving cognitive or motor activity. Hence, fractions with reduced albumin concentration, such as those fractions having below 83% albumin, are contemplated by the invention.

### 8. Treatment

Aspects of the methods of the inventions described herein include treatment of a subject with a plasma comprising blood product, such as a blood plasma fraction, e.g., as described above. An embodiment includes treatment of a human subject with a plasma comprising blood product. One of skill in the art would recognize that methods of treatment of subjects with plasma comprising blood products are recognized in the art. By way of example, and not limitation, one embodiment of the methods of the inventions described herein is comprised of administering fresh frozen plasma to a subject for treatment and/or prevention of cognitive or motor impairment, neuroinflammation, neurodegeneration, or peripheral diseases. In one embodiment, the plasma comprising blood product is administered immediately, e.g., within about 12-48 hours of collection from a donor, to the individual suffering or at risk from a cognitive or motor impairment, neuroinflammation, neurodegeneration, and/or age-related dementia. In such instances, the product may be stored under refrigeration, e.g., 0-10° C. In another embodiment, fresh frozen plasma is one that has been stored frozen (cryopreserved) at −18° C. or colder. Prior to administration, the fresh frozen plasma is thawed and once thawed, administered to a subject 60-75 minutes after the thawing process has begun. Each subject preferably receives a single unit of fresh frozen plasma (200-250 mL), the fresh frozen plasma preferably derived from donors of a pre-determined age range. In one embodiment of the invention, the fresh frozen plasma is donated by (derived from) young individuals. In another embodiment of the invention, the fresh frozen plasma is donated by (derived from) donors of the same gender. In another embodiment of the invention, the fresh frozen plasma is donated by (derived from) donors of the age range between 18-22 years old.

In an embodiment of the invention, the plasma comprising blood products are screened after donation by blood type. In another embodiment of the invention, the plasma comprising blood products are screened for infectious disease agents such as HIV I & II, HBV, HCV, HTLV I & II, anti-HBc per the requirements of 21 CFR 640.33 and recommendations contained in FDA guidance documents.

In yet another embodiment of the invention, the subject is treated with a Plasma Fraction. In an embodiment of the invention, the Plasma Fraction is a Fraction IV-1 paste (or cryoprecipitate) redissolved in an aqueous solution. “Fraction IV-1 paste” as used infra, is synonymous to Fraction IV-1 paste redissolved in an aqueous solution. In another embodiment of the invention, the Plasma Fraction can be a fractionated subfraction of Fraction IV-1 paste. In another embodiment of the invention, the Plasma Fraction can be one of the thirteen (13) subfractions of Fraction IV-1 paste described further infra. In another embodiment of the invention, the Plasma Fraction is PPF or HAS. In a further embodiment of the invention, the plasma fraction is one of the Commercial PPF Preparations of the Commercial HAS Preparations. In another embodiment of the invention the plasma fraction is a PPF or HAS derived from a pool of individuals of a specific age range, such as young individuals, or is a modified PPF or HAS fraction which has been subjected to additional fractionation or processing (e.g., PPF or HAS with one or more specific proteins partially or substantially removed). In another embodiment of the invention, the plasma fraction is an IGIV plasma fraction which has been substantially depleted of immune globulin (IgG). A blood fraction which is “substantially depleted” or which has specific proteins “substantially removed,” such as IgG, refers to a blood fraction containing less than about 50% of the amount that occurs in the reference product or whole blood plasma, such as less than 45%, 40%, 35%, 30%, 25%, 20%, 15%, 5%, 4%, 3%, 2%, 1%, 0.5%, 0.25%, 0.1%, undetectable levels, or any integer between these values, as measured using standard assays well known in the art.

### 9. Administration

Aspects of the methods of the inventions described herein include treatment of a subject with a plasma comprising blood product, such as a blood plasma or Plasma Fraction, e.g., as described above. An embodiment includes treatment of a human subject with a plasma comprising blood product.

In yet another embodiment of the invention, the subject is treated with a Plasma Fraction. Another embodiment of the invention includes treatment of a human subject with Fraction IV-1 paste. Another embodiment of the invention includes treatment of a human subject with a subfraction of Fraction IV-1 paste, including by way of example and not limitation, the third (13) subfractions further discussed infra. In an embodiment of the invention, the plasma fraction is PPF or HAS. In a further embodiment of the invention, the plasma fraction is one of the Commercial PPF Preparations of the Commercial HAS Preparations. In another embodiment of the invention the plasma fraction is a PPF or HAS derived from a pool of individuals of a specific age range, such as young individuals, or is a modified PPF or HAS fraction which has been subjected to additional fractionation or processing (e.g., PPF or HAS with one or more specific proteins partially or substantially removed). In another embodiment of the invention, the plasma fraction is an IGIV plasma fraction which has been substantially depleted of immune globulin (IgG). A blood fraction which is “substantially depleted” or which has specific proteins “substantially removed,” such as IgG, refers to a blood fraction containing less than about 50% of the amount that occurs in the reference product or whole blood plasma, such as less than 45%, 40%, 35%, 30%, 25%, 20%, 15%, 5%, 4%, 3%, 2%, 1%, 0.5%, 0.25%, 0.1%, undetectable levels, or any integer between these values, as measured using standard assays well known in the art.

An embodiment of the invention includes treating a subject diagnosed with a cognitive or motor impairment, neurodegeneration, neuroinflammation, or peripheral disease by administering to the subject an effective amount of blood plasma or Plasma Fraction. Another embodiment of the invention includes administering the effective amount of blood plasma or Plasma Fraction and subsequently monitoring the subject for improvement in the disease symptoms. Another embodiment of the invention includes treating a subject diagnosed with a disease or disorder by administering to the subject an effective amount of blood plasma or Plasma Fraction wherein the blood plasma or Plasma Fraction is administered in a manner resulting improvement in the symptoms or progression of the disease after the mean or median half-life of the blood plasma proteins or Plasma Fraction proteins been reached, relative to the most recent administered dose (referred to as “Pulsed Dosing” or “Pulse Dosed” herein). Another embodiment of the invention includes administering the blood plasma or Plasma Fraction via a dosing regimen of at least two consecutive days and monitoring the subject for improved cognitive or motor function, decreased neuroinflammation or improved neurogenesis at least 3 days after the date of last administration. A further embodiment of the invention includes administering the blood plasma or Plasma Fraction via a dosing regimen of at least 3, 4, 5, 6, 7, 8, 9, 10, 11, 12, 13, or 14 consecutive days and monitoring the subject for improvement in disease symptoms or progression at least 3 days after the date of last administration. Yet another embodiment of the invention includes administering the blood plasma or Plasma Fraction via a dosing regimen of at least 2 consecutive days and after the date of last administration. Another embodiment of the invention includes administering the blood plasma or Plasma Fraction via a dosing regimen of 2 to 14 non-consecutive days wherein each gap between doses may be between 0-3 days each.

In some instances, Pulsed Dosing in accordance with the invention includes administration of a first set of doses, e.g., as described above, followed by a period of no dosing, e.g., a “dosing-free period”, which in turn is followed by administration of another dose or set of doses. The duration of this “dosing-free” period, may vary, but in some embodiments, is 7 days or longer, such as 10 days or longer, including 14 days or longer, wherein some instances the dosing-free period ranges from 15 to 365 days, such as 30 to 90 days and including 30 to 60 days. As such, embodiments of the methods include non-chronic (i.e., non-continuous) dosing, e.g., non-chronic administration of a blood plasma product. In some embodiments, the pattern of Pulsed Dosing followed by a dosing-free period is repeated for a number of times, as desired, where in some instances this pattern is continued for 1 year or longer, such as 2 years or longer, up to and including the life of the subject. Another embodiment of the invention includes administering the blood plasma or Plasma Fraction via a dosing regimen of 5 consecutive days, with a dosing-free period of 2-3 days, followed by administration for 2-14 consecutive days.

Biochemically, by an “effective amount” or “effective dose” of active agent is meant an amount of active agent that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances, reverse the progression of the disease.

### 10. Plasma Protein Fraction

In practicing methods of the invention, a plasma fraction is administered to the subject. In an embodiment, the plasma fraction is plasma protein fraction (PPF). In additional embodiments, the PPF is selected from the Commercial PPF Preparations.

In another embodiment, the PPF is comprised of 88% normal human albumin, 12% alpha and beta globulins and not more than 1% gamma globulin as determined by electrophoresis. Further embodiments of this embodiment used in practicing methods of the invention include, for example, the embodiment as a 5% solution of PPF buffered with sodium carbonate and stabilized with 0.004 M sodium caprylate and 0.004 M acetyltryptophan. Additional formulations, including those modifying the percentage of PPF (e.g. about 1% to about 10%, about 10% to about 20%, about 20% to 25%, about 25% to 30%) in solution as well as the concentrations of solvent and stabilizers may be utilized in practicing methods of the invention.

### 11. Indications

The subject methods and plasma-comprising blood products and Plasma Fractions find use in treating, including preventing, aging-associated conditions, such as impairments in the cognitive or motor ability of individuals, e.g., cognitive disorders, including (but not limited to) age-associated dementia, immunological conditions, cancer, and physical and functional decline; and motor disorders such as (but not limited to) Parkinson's disease. Individuals suffering from or at risk of developing an aging-associated cognitive or motor impairment, neuroinflammation, and/or neurodegeneration that will benefit from treatment with the subject plasma-comprising blood product, e.g., by the methods disclosed herein, include individuals that are about 50 years old or older, e.g., 60 years old or older, 70 years old or older, 80 years old or older, 90 years old or older, and 100 years old or older, i.e., between the age of about 50 and 100, e.g., 50, 55, 60, 65, 70, 75, 80, 85, 90, 95 or about 100 years old, and are suffering from cognitive or motor impairment, neuroinflammation, and/or neurodegeneration associated with natural aging process, e.g., mild cognitive impairment (M.C.I.); and individuals that are about 50 years old or older, e.g., 60 years old or older, 70 years old or older, 80 years old or older, 90 years old or older, and usually no older than 100 years old, i.e., between the ages of about 50 and 90, e.g., 50, 55, 60, 65, 70, 75, 80, 85, 90, 95 or about 100 years old, that have not yet begun to show symptoms of cognitive or motor impairment, neuroinflammation and/or neurodegeneration. Examples of cognitive and motor, neuroinflammatory, and/or neurodegenerative impairments that are due to natural aging include the following:
a. Mild cognitive impairment (M.C.I.). Mild cognitive impairment is a modest disruption of cognition that manifests as problems with memory or other mental functions such as planning, following instructions, or making decisions that have worsened over time while overall mental function and daily activities are not impaired. Thus, although significant neuronal death does not typically occur, neurons in the aging brain are vulnerable to sub-lethal age-related alterations in structure, synaptic integrity, and molecular processing at the synapse, all of which impair cognitive function.

Individuals suffering from or at risk of developing an aging-associated cognitive impairment that will benefit from treatment with the subject plasma-comprising blood product or fraction, e.g., by the methods disclosed herein, also include individuals of any age that are suffering from a cognitive impairment due to an aging-associated disorder; and individuals of any age that have been diagnosed with an aging-associated disorder that is typically accompanied by cognitive impairment, where the individual has not yet begun to present with symptoms of cognitive impairment. Examples of such aging-associated disorders include the following:
b. Alzheimer's disease. Alzheimer's disease is a progressive, inexorable loss of cognitive function associated with an excessive number of senile plaques in the cerebral cortex and subcortical gray matter, which also contains b-amyloid and neurofibrillary tangles consisting of tau protein. The common form affects persons>60 yr old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, synapses, and ultimately neurons are lost within the cerebral cortex, hippocampus, and subcortical structures (including selective cell loss in the nucleus basalis of Meynert), locus coeruleus, and nucleus raphe dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

### c. Parkinson's Disease

Parkinson's Disease (PD) is an idiopathic, slowly progressive, degenerative CNS disorder characterized by slow and decreased movement (bradykinesia), muscular rigidity, resting tremor (dystonia), muscle freezing, and postural instability. Originally considered primarily a motor disorder, PD is now recognized to also cause depression and emotional changes. PD also can affect cognition, behavior, sleep, autonomic function, and sensory function. The most common cognitive impairments include an impairment in attention and concentration, working memory, executive function, producing language, and visuospatial function. A characteristic of PD is symptoms related to reduced motor function usually precede those related to cognitive impairment, which aids in diagnosis of the disease.

In primary Parkinson's disease, the pigmented neurons of the substantia nigra, locus cocruleus, and other brain stem dopaminergic cell groups degenerate. The cause is not known. The loss of substantia nigra neurons, which project to the caudate nucleus and putamen, results in depletion of the neurotransmitter dopamine in these areas. Onset is generally after age 40, with increasing incidence in older age groups.

Parkinson's disease is newly diagnosed in about 60,000 Americans each year and currently affects approximately one million Americans. Even though PD is not fatal in itself, its complications are the fourteenth leading cause of death in the United States. At present, PD cannot be cured, and treatment is generally prescribed to control symptoms, with surgery prescribed in later, severe cases.

Treatment options for PD include administration of pharmaceuticals to help manage motor deficits. These options increase or substitute for the neurotransmitter, dopamine, of which PD patients have low brain concentrations. Such medications include: carbidopa/levodopa (which create more dopamine in the brain); apomorphine, pramipexole, ropinirole, and rotingotine (dopamine agonists); selegiline and rasagiline (MAO-B inhibitors which prevent breakdown of dopamine); entacapone and tolcapone (Catechol-O-methyltransferase [COMT] inhibitors which make more levodopa available in the brain); benztropine and trihexyphenidyl (anticholinergics); and amantadine (controls tremor and stiffness). Exercise/physical therapy is also commonly prescribed to help maintain physical and mental function.

Current treatment options, however treat the symptoms of PD, are not curative, and fail to prevent disease progression. Additionally, current medications tend to lose efficacy in late stage PD. The most prescribed drug, levodopa, commonly results in adverse effects within 5 to 10 years after commencing the medication. These adverse effects can be severe and can result in motor fluctuations and unpredictable swings in motor control between doses as well as jerking/twitching (dyskinesia) which are difficult to manage and are even as disabling as PD's own symptoms. Thus, there remains a need for new therapies with new mechanisms of action which can either be administrated along or in combination with current PD medications.
d. Parkinsonism. Secondary parkinsonism (also referred to as atypical Parkinson's disease or Parkinson's plus) results from loss of or interference with the action of dopamine in the basal ganglia due to other idiopathic degenerative diseases, drugs, or exogenous toxins. The most common cause of secondary parkinsonism is ingestion of antipsychotic drugs or reserpine, which produce parkinsonism by blocking dopamine receptors. Less common causes include carbon monoxide or manganese poisoning, hydrocephalus, structural lesions (tumors, infarcts affecting the midbrain or basal ganglia), subdural hematoma, and degenerative disorders, including nigrostriatal degeneration. Certain disorders like Progressive Supranuclear Palsy (PSP), Multiple System Atrophy (MSA), Corticobasal degeneration (CBD) and Dementia with Lewy Bodies (DLB) can exhibit Parkinsonism symptoms before the cardinal symptoms necessary to the specific diagnosis can be made, and thus may be labeled as “Parkinsonism.”
e. Frontotemporal dementia. Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. Symptoms are classified into three groups based on the functions of the frontal and temporal lobes affected:

Behavioral variant FTD (bvFTD), with symptoms include lethargy and aspontaneity on the one hand, and disinhibition on the other; progressive nonfluent aphasia (PNFA), in which a breakdown in speech fluency due to articulation difficulty, phonological and/or syntactic errors is observed but word comprehension is preserved; and semantic dementia (SD), in which patients remain fluent with normal phonology and syntax but have increasing difficulty with naming and word comprehension. Other cognitive symptoms common to all FTD patients include an impairment in executive function and ability to focus. Other cognitive abilities, including perception, spatial skills, memory and praxis typically remain intact. FTD can be diagnosed by observation of reveal frontal lobe and/or anterior temporal lobe atrophy in structural MRI scans.

A number of forms of FTD exist, any of which may be treated or prevented using the subject methods and compositions. For example, one form of frontotemporal dementia is Semantic Dementia (SD). SD is characterized by a loss of semantic memory in both the verbal and non-verbal domains. SD patients often present with the complaint of word-finding difficulties. Clinical signs include fluent aphasia, anomia, impaired comprehension of word meaning, and associative visual agnosia (the inability to match semantically related pictures or objects). As the disease progresses, behavioral and personality changes are often seen similar to those seen in frontotemporal dementia although cases have been described of ‘pure’ semantic dementia with few late behavioral symptoms. Structural MRI imaging shows a characteristic pattern of atrophy in the temporal lobes (predominantly on the left), with inferior greater than superior involvement and anterior temporal lobe atrophy greater than posterior.

As another example, another form of frontotemporal dementia is Pick's discasc (PiD, also PcD). A defining characteristic of the disease is build-up of tau proteins in neurons, accumulating into silver-staining, spherical aggregations known as “Pick bodies.” Symptoms include loss of speech (aphasia) and dementia. Patients with orbitofrontal dysfunction can become aggressive and socially inappropriate. They may steal or demonstrate obsessive or repetitive stereotyped behaviors. Patients with dorsomedial or dorsolateral frontal dysfunction may demonstrate a lack of concern, apathy, or decreased spontaneity. Patients can demonstrate an absence of self-monitoring, abnormal self-awareness, and an inability to appreciate meaning. Patients with gray matter loss in the bilateral posterolateral orbitofrontal cortex and right anterior insula may demonstrate changes in eating behaviors, such as a pathologic sweet tooth. Patients with more focal gray matter loss in the anterolateral orbitofrontal cortex may develop hyperphagia. While some of the symptoms can initially be alleviated, the disease progresses and patients often die within two to ten years.
f. Huntington's disease. Huntington's disease (HD) is a hereditary progressive neurodegenerative disorder characterized by the development of emotional, behavioral, and psychiatric abnormalities; loss of intellectual or cognitive functioning; and movement abnormalities (motor disturbances). The classic signs of HD include the development of chorea-involuntary, rapid, irregular, jerky movements that may affect the face, arms, legs, or trunk—as well as cognitive decline including the gradual loss of thought processing and acquired intellectual abilities. There may be impairment of memory, abstract thinking, and judgment; improper perceptions of time, place, or identity (disorientation); increased agitation; and personality changes (personality disintegration). Although symptoms typically become evident during the fourth or fifth decades of life, the age at onset is variable and ranges from early childhood to late adulthood (e.g., 70s or 80s).

HD is transmitted within families as an autosomal dominant trait. The disorder occurs as the result of abnormally long sequences or “repeats” of coded instructions within a gene on chromosome 4 (4p16.3). The progressive loss of nervous system function associated with HD results from loss of neurons in certain areas of the brain, including the basal ganglia and cerebral cortex.
g. Amyotrophic lateral sclerosis. Amyotrophic lateral sclerosis (ALS) is a rapidly progressive, invariably fatal, neurological disease that attacks motor neurons. Muscular weakness and atrophy and signs of anterior horn cell dysfunction are initially noted most often in the hands and less often in the feet. The site of onset is random, and progression is asymmetric. Cramps are common and may precede weakness. Rarely, a patient survives 30 years; 50% die within 3 years of onset, 20% live 5 years, and 10% live 10 years.

Diagnostic features include onset during middle or late adult life and progressive, generalized motor involvement without sensory abnormalities. Nerve conduction velocities are normal until late in the disease. Recent studies have documented the presentation of cognitive impairments as well, particularly a reduction in immediate verbal memory, visual memory, language, and executive function.

A decrease in cell body area, number of synapses and total synaptic length has been reported in even normal-appearing neurons of the ALS patients. It has been suggested that when the plasticity of the active zone reaches its limit, a continuing loss of synapses can lead to functional impairment. Promoting the formation or new synapses or preventing synapse loss may maintain neuron function in these patients.
h. Multiple Sclerosis. Multiple Sclerosis (MS) is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. Common cognitive impairments include impairments in memory (acquiring, retaining, and retrieving new information), attention and concentration (particularly divided attention), information processing, executive functions, visuospatial functions, and verbal fluency. Common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive.
i. Glaucoma. Glaucoma is a common neurodegenerative disease that affects retinal ganglion cells (RGCs). Evidence supports the existence of compartmentalized degeneration programs in synapses and dendrites, including in RGCs. Recent evidence also indicates a correlation between cognitive impairment in older adults and glaucoma (Yochim B P, et al. Prevalence of cognitive impairment, depression, and anxiety symptoms among older adults with glaucoma. J Glaucoma. 2012; 21 (4): 250-254).
j. Myotonic dystrophy. Myotonic dystrophy (DM) is an autosomal dominant multisystem disorder characterized by dystrophic muscle weakness and myotonia. The molecular defect is an expanded trinucleotide (CTG) repeat in the 3′ untranslated region of the myotoninprotein kinase gene on chromosome 19q. Symptoms can occur at any age, and the range of clinical severity is broad. Myotonia is prominent in the hand muscles, and ptosis is common even in mild cases. In severe cases, marked peripheral muscular weakness occurs, often with cataracts, premature balding, hatchet facies, cardiac arrhythmias, testicular atrophy, and endocrine abnormalities (e.g., diabetes mellitus). Mental retardation is common in severe congenital forms, while an aging-related decline of frontal and temporal cognitive functions, particularly language and executive functions, is observed in milder adult forms of the disorder. Severely affected persons die by their early 50s.
k. Dementia. Dementia describes a class of disorders having symptoms affecting thinking and social abilities severely enough to interfere with daily functioning. Other instances of dementia in addition to the dementia observed in later stages of the aging-associated disorders discussed above include vascular dementia, and dementia with Lewy bodies, described below.

In vascular dementia, or “multi-infarct dementia”, cognitive impairment is caused by problems in supply of blood to the brain, typically by a series of minor strokes, or sometimes, one large stroke preceded or followed by other smaller strokes. Vascular lesions can be the result of diffuse cerebrovascular disease, such as small vessel disease, or focal lesions, or both. Patients suffering from vascular dementia present with cognitive impairment, acutely or subacutely, after an acute cerebrovascular event, after which progressive cognitive decline is observed. Cognitive impairments are similar to those observed in Alzheimer's disease, including impairments in language, memory, complex visual processing, or executive function, although the related changes in the brain are not due to AD pathology but to chronic reduced blood flow in the brain, eventually resulting in dementia. Single photon emission computed tomography (SPECT) and positron emission tomography (PET) neuroimaging may be used to confirm a diagnosis of multi-infarct dementia in conjunction with evaluations involving mental status examination.

Dementia with Lewy bodies (DLB, also known under a variety of other names including Lewy body dementia, diffuse Lewy body disease, cortical Lewy body disease, and senile dementia of Lewy type) is a type of dementia characterized anatomically by the presence of Lewy bodies (clumps of alpha-synuclein and ubiquitin protein) in neurons, detectable in post mortem brain histology. Its primary feature is cognitive decline, particularly of executive functioning. Alertness and short term memory will rise and fall.

Persistent or recurring visual hallucinations with vivid and detailed pictures are often an early diagnostic symptom. DLB it is often confused in its early stages with Alzheimer's disease and/or vascular dementia, although, where Alzheimer's disease usually begins quite gradually, DLB often has a rapid or acute onset. DLB symptoms also include motor symptoms similar to those of Parkinson's. DLB is distinguished from the dementia that sometimes occurs in Parkinson's disease by the time frame in which dementia symptoms appear relative to Parkinson symptoms. Parkinson's disease with dementia (POD) would be the diagnosis when dementia onset is more than a year after the onset of Parkinson's. DLB is diagnosed when cognitive symptoms begin at the same time or within a year of Parkinson symptoms.
l. Progressive supranuclear palsy. Progressive supranuclear palsy (PSP) is a brain disorder that causes serious and progressive problems with control of gait and balance, along with complex cyc movement and thinking problems. One of the classic signs of the disease is an inability to aim the eyes properly, which occurs because of lesions in the area of the brain that coordinates eye movements. Some individuals describe this effect as a blurring. Affected individuals often show alterations of mood and behavior, including depression and apathy as well as progressive mild dementia. The disorder's long name indicates that the disease begins slowly and continues to get worse (progressive), and causes weakness (palsy) by damaging certain parts of the brain above pea-sized structures called nuclei that control eye movements (supranuclear). PSP was first described as a distinct disorder in 1964, when three scientists published a paper that distinguished the condition from Parkinson's disease. It is sometimes referred to as Steele-Richardson-Olszewski syndrome, reflecting the combined names of the scientists who defined the disorder. Although PSP gets progressively worse, no one dies from PSP itself.
m. Ataxia. People with ataxia have problems with coordination because parts of the nervous system that control movement and balance are affected. Ataxia may affect the fingers, hands, arms, legs, body, speech, and eye movements. The word ataxia is often used to describe a symptom of incoordination which can be associated with infections, injuries, other diseases, or degenerative changes in the central nervous system. Ataxia is also used to denote a group of specific degenerative diseases of the nervous system called the hereditary and sporadic ataxias which are the National Ataxia Foundation's primary emphases.
n. Multiple-system atrophy. Multiple-system atrophy (MSA) is a degenerative neurological disorder. MSA is associated with the degeneration of nerve cells in specific areas of the brain. This cell degeneration causes problems with movement, balance, and other autonomic functions of the body such as bladder control or blood-pressure regulation.

The cause of MSA is unknown and no specific risk factors have been identified. Around 55% of cases occur in men, with typical age of onset in the late 50s to early 60s. MSA often presents with some of the same symptoms as Parkinson's disease. However, MSA patients generally show minimal if any response to the dopamine medications used for Parkinson's.
o. Frailty. Frailty Syndrome (“Frailty”) is a geriatric syndrome characterized by functional and physical decline including decreased mobility, muscle weakness, physical slowness, poor endurance, low physical activity, malnourishment, and involuntary weight loss. Such decline is often accompanied and a consequence of diseases such as cognitive dysfunction and cancer. However, Frailty can occur even without discasc. Individuals suffering from Frailty have an increased risk of negative prognosis from fractures, accidental falls, disability, comorbidity, and premature mortality. (C. Buigues, et al. Effect of a Prebiotic Formulation on Frailty Syndrome: A Randomized, Double-Blind Clinical Trial, Int. J. Mol. Sci. 2016, 17, 932). Additionally, individuals suffering from Frailty have an increased incidence of higher health care expenditure. (Id.)

Common symptoms of Frailty can be determined by certain types of tests. For example, unintentional weight loss involves a loss of at least 10 lbs, or greater than 5% of body weight in the preceding year; muscle weakness can be determined by reduced grip strength in the lowest 20% at baseline (adjusted for gender and BMI); physical slowness can be based on the time needed to walk a distance of 15 feet; poor endurance can be determined by the individual's self-reporting of exhaustion; and low physical activity can be measured using a standardized questionnaire. (Z. Palace et al., The Frailty Syndrome, Today's Geriatric Medicine 7 (1), at 18 (2014)).

In some embodiments, the subject methods and compositions find use in slowing the progression of aging-associated cognitive, motor, neuroinflammatory, or other age-related impairment or condition. In other words, cognitive, motor, neuroinflammatory, or other abilities or conditions in the individual will decline more slowly following treatment by the disclosed methods than prior to or in the absence of treatment by the disclosed methods. In some such instances, the subject methods of treatment include measuring the progression of cognitive, motor, neuroinflammation, or other age-related ability or symptom decline after treatment, and determining that the progression of decline is reduced. In some such instances, the determination is made by comparing to a reference, e.g., the rate of decline in the individual prior to treatment, e.g., as determined by measuring cognitive, motor, neuroinflammatory, or other age-related abilities or conditions prior at two or more time points prior to administration of the subject blood product.

The subject methods and compositions also find use in stabilizing the cognitive, motor, neuroinflammatory, or other abilities or conditions of an individual, e.g., an individual suffering from aging-associated cognitive decline or an individual at risk of suffering from aging-associated cognitive decline. For example, the individual may demonstrate some aging-associated cognitive impairment, and progression of cognitive impairment observed prior to treatment with the disclosed methods will be halted following treatment by the disclosed methods. As another example, the individual may be at risk for developing an aging-associated cognitive declinc (e.g., the individual may be aged 50 years old or older, or may have been diagnosed with an aging-associated disorder), and the cognitive abilities of the individual are substantially unchanged, i.e., no cognitive decline can be detected, following treatment by the disclosed methods as compared to prior to treatment with the disclosed methods.

The subject methods and compositions also find use in reducing cognitive, motor, neuroinflammatory, or other age-related impairment in an individual suffering from an aging-associated impairment. In other words, the affected ability is improved in the individual following treatment by the subject methods. For example, the cognitive or motor ability in the individual is increased, e.g., by 2-fold or more, 5-fold or more, 10-fold or more, 15-fold or more, 20-fold or more, 30-fold or more, or 40-fold or more, including 50-fold or more, 60-fold or more, 70-fold or more, 80-fold or more, 90-fold or more, or 100-old or more, following treatment by the subject methods relative to the cognitive or motor ability that is observed in the individual prior to treatment by the subject methods.

In some instances, treatment by the subject methods and compositions restores the cognitive, motor, or other ability in the individual suffering from aging-associated cognitive or motor decline, e.g., to their level when the individual was about 40 years old or less. In other words, cognitive or motor impairment is abrogated. Methods of Diagnosing and Monitoring for Improvement
12. In some instances, among the variety of methods to diagnose and monitor disease progression and improvement in cognitive disease, motor impairment, neurodegenerative disease, and/or neuroinflammatory disease the following types of assessments are used alone or in combination with subjects suffering from neurodegenerative disease, as desired. The following types of methods are presented as examples and are not limited to the recited methods. Any convenient methods to monitor disease may be used in practicing the invention, as desired. Those methods are also contemplated by the methods of the invention.
   a. General Cognition

Embodiments of the methods of the invention further comprise methods of monitoring the effect of a medication or treatment on a subject for treating cognitive impairment and/or age-related dementia, the method comprising comparing cognitive function before and after treatment. Those having ordinary skill in the art recognize that there are well-known methods of evaluating cognitive function. For example, and not by way of limitation, the method may comprise evaluation of cognitive function based on medical history, family history, physical and neurological examinations by clinicians who specialize dementia and cognitive function, laboratory tests, and neuropsychological assessment. Additional embodiments which are contemplated by the invention include: the assessment of consciousness, such as using the Glasgow Coma Scale (EMV); mental status examination, including the abbreviated mental test score (AMTS) or mini-mental state examination (MMSE) (Folstein et al., J. Psychiatr. Res 1975; 12:1289-198); global assessment of higher functions; estimation of intracranial pressure such as by fundoscopy. In one embodiment, monitoring the effect on cognitive impairment and/or age-related dementia includes a 1, 2, 3, 4, 5, 6, 7, 8, 9, 10, 11, or 12-point improvement using the Alzheimer's Disease Assessment Scale-Cognitive Subscale (ADAS-COG).

In one embodiment, examinations of the peripheral nervous system may be used to evaluate cognitive function, including any one of the followings: sense of smell, visual fields and acuity, eye movements and pupils (sympathetic and parasympathetic), sensory function of face, strength of facial and shoulder girdle muscles, hearing, taste, pharyngeal movement and reflex, tongue movements, which can be tested individually (e.g. the visual acuity can be tested by a Snellen chart; a reflex hammer used testing reflexes including masseter, biceps and triceps tendon, knee tendon, ankle jerk and plantar (i.e. Babinski sign); Muscle strength often on the MRC scale 1 to 5; Muscle tone and signs of rigidity.

b. Parkinson's Disease

Embodiments of the methods of the invention further comprise methods of monitoring the effect of a medication or treatment on a subject for treating motor impairment, the method comprising comparing motor function before and after treatment. Those having ordinary skill in the art recognize that there are well-known methods of evaluating motor function. For example, and not by way of limitation, the method may comprise evaluation of motor function based on medical history, family history, physical and neurological examinations by clinicians who specialize neurodegeneration and motor impairment, laboratory tests, and neurodegenerative assessment. Additional embodiments which are contemplated by the invention include employment of the rating scales discussed below.

Several rating scales have been utilized for evaluating the progression of PD. The most widely-used scales include the Unified Parkinson's Disease Rating Scale (UPDRS, which was introduced in 1987) (J. Rchabil Rcs. Dev., 2012 49 (8): 1269-76), and the Hochn and Yahr scale (Neruology, 1967 17 (5): 427-42). Additional scales include the Movement Disorder Society (MDS)'s updated UPDRS scale (MDS-UPDRS) as well as the Schwab and England Activities of Daily Living (ADL) Scale.

The UPDRS scale evaluates 31 items that contributed to three subscales: (1) mentation, behavior, and mood; (2) activities of daily living; and (3) motor examination. The Hoehn and Yahr scale classifies PD into five stages with discreet substages: 0—no signs of disease; 1—symptoms on one side only; 1.5—symptoms on one side but also involving neck and spine; 2—symptoms on both sides with no balance impairment; 2.5—mild symptoms on both sides, with recovery when the ‘pull’ test is given; 3—balance impairment with mild to moderate disease; 4—severe disability, but ability to walk or stand unassisted; and 5—need a wheelchair or bedridden without assistance. The Schwab and England scale classifies PD into several percentages (from 100%—complete independent to 10%—total dependent).

General motor function can be evaluated using widely-used scales including the General Motor Function Scale (GMF). This tests three components: dependence, pain, and insecurity. (Aberg A. C., et al. (2003) Disabil. Rehabil. 2003 May 6; 25 (9): 462-72.). Motor function can also be assessed using home-monitoring or wearable sensors. For example: gait (speed of locomotion, variability, leg rigidity) can be sensed with an accelerometer; posture (trunk inclination) by a gyroscope; leg movement by an accelerometer; hand movement by an accelerometer and gyroscope; tremor (amplitude, frequency, duration, asymmetry) by an accelerometer; falling by an accelerometer; gait freezing by an accelerometer; dyskinesia by an accelerometer, gyroscope, and inertial sensors; bradykinesia (duration and frequency) by an accelerometer plus gyroscope, and aphasia (pitch) using a microphone. (Pastorino M, et al., Journal of Physics: Conference Series 450 (2013) 012055).

c. Multiple Sclerosis

In addition to monitoring improvement for symptoms associated with cognition, the progression or improvement of neurodegeneration associated with multiple sclerosis (MS) can be monitored using techniques well-known to those having ordinary skill in the art. By way of example, and not limitation, monitoring can be performed through techniques such as: cerebrospinal fluid (CSF) monitoring; magnetic resonance imaging (MRI) to detect lesions and development of demyelinating plaques; evoked potential studies; and gait monitoring.

CSF analysis may be performed, for example, through lumbar puncture to obtain pressure, appearance, and CSF content. Normal values typically range as follows: pressure (70-180 mm H20); appearance is clear and colorless; total protein (15-60 mg/100 mL); IgG is 3-12% of the total protein; glucose is 50-80 mg/100 mL; cell count is 0-5 white blood cells and no red blood cells; chloride (110-125 mEq/L). Abnormal results may indicate the presence or progression of MS.

MRI is another technique that may be performed to monitor disease progression and improvement. Typical criteria for monitoring MS with MRI include the appearance of patchy areas of abnormal white matter in cerebral hemisphere and in paraventricular areas, lesions present in the cerebellum and/or brain stem as well as in the cervical or thoracic regions of the spinal cord.

Evoked potentials may be used to monitor the progression and improvement of MS in subjects. Evoked potentials measure slowing of electrical impulses such as in Visual Evoked Response (VER), Brain Stem Auditory Evoked Responses (BAER), and Somatosensory Evoked Responses (SSER). Abnormal responses help to indicate that there is a decrease in the speed of conduction in central sensory pathways.

Gait monitoring can also be used to monitor disease progression and improvement in MS subjects. MS is often accompanied by an impairment in mobility and an abnormal gait due in part to fatigue. Monitoring may be performed, for example, with the use of mobile monitoring devices worn by subjects. (Moon, Y., et al., Monitoring gait in multiple sclerosis with novel wearable motion sensors, PLOS One, 12 (2): e0171346 (2017)).

d. Huntington's

In addition to monitoring improvement for symptoms associated with cognition, the progression or improvement of neurodegeneration associated with Huntington's Disease (HD) can be monitored using techniques well-known to those having ordinary skill in the art. By way of example, and not limitation, monitoring can be performed through techniques such as: motor function; behavior; functional assessment; and imaging.

Examples of motor function that may be monitored as an indication of disease progression or improvement include chorea and dystonia, rigidity, bradykinesia, oculomotor dysfunction, and gait/balance changes. Techniques for performing the monitoring of these metrics are well-known to those having ordinary skill in the art. (See Tang C, et al., Monitoring Huntington's disease progression through preclinical and early stages, Neurodegener Dis Manag 2 (4): 421-35 (2012)).

The psychiatric effects of HD present opportunities to monitor disease progression and improvement. For example, psychiatric diagnoses may be performed in order to determine whether the subject suffers from depression, irritability, agitation, anxiety, apathy and psychosis with paranoia. (Id.)

Functional assessment may also be employed to monitor disease progression or improvement. Total functional score techniques have been reported (Id.), and often declines by one point per year in some HD groups.

MRI or PET may be employed also to monitor disease progression or improvement. For example, there is a loss of striatal projection neurons in HD, and change in number of these neurons may be monitored in subjects. Techniques to determine neuronal change in HD subjects include imaging Dopaminc D2 receptor binding. (Id.)

e. ALS

In addition to monitoring improvement for symptoms associated with cognition, the progression or improvement of neurodegeneration associated with Amyotrophic Lateral Sclerosis (ALS) can be monitored using techniques well-known to those having ordinary skill in the art. By way of example, and not limitation, monitoring can be performed through techniques such as: functional assessment; determining muscle strength; measuring respiratory function; measuring lower motor neuron (LMN) loss; and measuring upper motor neuron (UMN) dysfunction.

Functional assessment can be performed using a functional scale well-known to those having ordinary skill in the art, such as the ALS Functional Rating Scale (ALSFRS-R), which evaluates symptoms related to bulbar, limb, and respiratory function. The rate of change is useful in predicting survival as well as disease progression or improvement. Another measure includes the Combined Assessment of Function and Survival (CAFS), ranking subjects' clinical outcomes by combining survival time with change in ALSFRS-R. (Simon N G, et al., Quantifying Disease Progression in Amyotrophic Lateral Sclerosis, Ann Neurol 76:643-57 (2014)).

Muscle strength may be tested and quantified through use of composite Manual Muscle Testing (MMT) scoring. This entails averaging measures acquired from several muscle groups using the Medical Research Council (MRC) muscle strength grading scale. (Id.) Hand-held dynamometry (HHD) may also be used, among other techniques. (Id.)

Respiratory function can be performed using portable spirometry units, used to obtain Forced Vital Capacity (FVC) at baseline to predict the progression or improvement of the disease. Additionally, maximal inspiratory pressure, sniff nasal inspiratory pressure (SNIP), and supping FVC may be determined and used to monitor disease progression/improvement. (Id.)

Loss in lower motor neurons is another metric which can be utilized to monitor disease progression or improvement in ALS. The Neurophysiological Index may be determined by measuring compound muscle action potentials (CMAPs) on motor nerve conduction studies, of which parameters include CMAP amplitude and F-wave frequency. (Id. and de Carvalho M, et al., Nerve conduction studies in amyotrophic lateral sclerosis. Muscle Nerve 23:344-352, (2000)). Lower motor neuron unit numbers (MUNE) may be estimated as well. In MUNE, the number of residual motor axons supplying a muscle through estimation of the contribution of individual motor units to the maximal CMAP response is estimated, and used to determine discasc progression or improvement. (Simon N G, et al., supra). Additional techniques for determining loss of LMN include testing nerve excitability, electrical impedance myography, and using muscle ultrasound to detect changes in thickness in muscles. (Id.)

Dysfunction of upper motor neurons is another metric which can be utilized to monitor disease progression or improvement in ALS. Techniques for determining dysfunction include performing MRI or PET scans on the brain and spinal cord, transcranial magnetic stimulation; and determining levels of biomarkers in the cerebrospinal fluid (CSF).

f. Glaucoma

In addition to monitoring improvement for symptoms associated with cognition, the progression or improvement of neurodegeneration associated with glaucoma can be monitored using techniques well-known to those having ordinary skill in the art. By way of example, and not limitation, monitoring can be performed through techniques such as: determining intraocular pressure; assessment of the optic disc or optic nerve head for damage; visual field testing for peripheral vision loss; and imaging of the optic disc and retina for topographic analysis.

g. Progressive Supranuclear Palsy (PSP)

In addition to monitoring improvement for symptoms associated with cognition, the progression or improvement of neurodegeneration associated with Progressive Supranuclear Palsy (PSP) can be monitored using techniques well-known to those having ordinary skill in the art. By way of example, and not limitation, monitoring can be performed through techniques such as: functional assessment (activities of daily living, or ADL); motor assessment; determination of psychiatric symptoms; and volumetric and functional magnetic resonance imaging (MRI).

The level of function of a subject in terms of independence, partial dependence upon others, or complete dependence can be useful for determining the progression or improvement in the disease. (See Duff, K, et al., Functional impairment in progressive supranuclear palsy, Neurology 80:380-84, (2013)). The Progressive Supranuclear Palsy Rating Scale (PSPRS) is a rating scale that comprises twenty-eight metrics in six categories: daily activities (by history); behavior; bulbar, ocular motor, limb motor and gait/midline. The result is a score ranging from 0-100. Six items are graded 0-2 and twenty-two items graded 0-4 for a possible total of 100. The PSPRS scores are practical measures, and robust predictors of patient survival. They are also sensitive to disease progression and useful in monitoring disease progression or improvement. (Golbe L I, et al., A clinical rating scale for progressive supranuclear palsy, Brain 130:1552-65, (2007)).

The ADL section from the UPDRS (Unified Parkinson's Disease Rating Scale) can also be used to quantify functional activity in subjects with PSP. (Duff K, et al., supra). Similarly, the Schwab & England Activities Daily Living Score (SE-ADL) can be used for evaluate independence. (Id.) Additionally, the motor function sections of the UPDRS are useful as a reliable measure for assessing disease progression in PSP patients. The motor section may contain, for example, 27 different measures for quantifying motor function in PSP patients. Examples of these include resting tremor, rigidity, finger tapping, posture, and gait). A subject's disease progression or improvement may also be assessed by performing a baseline neuropsychological evaluation completed by trained medical personnel, the assessment using the Neuropsychiatric Inventory (NPI) to determine the frequency and severity of behavior abnormalities (e.g., delusions, hallucinations, agitation, depression, anxiety, euphoria, apathy, disinhibition, irritability, and aberrant motor behavior). (Id.)

Functional MRI (fMRI) can be employed to monitor disease progression and improvement as well. fMRI is a technique using MRI to measure changes in brain activity in certain regions of the brain, usually based on blood flow to those regions. Blood flow is considered to correlate with brain region activation. Patients with neurodegenerative disorders like PSP can be subjected to physical or mental tests before or during being scanned in an MRI scanner. By way of example, and not limitation, tests can be a well-established force control paradigm where patients as asked to produce force with the hand most affected by PSP and maximum voluntary contraction (MVC) is measured by fMRI immediately after the test takes place. Burciu, RG, ct al., Distinct patterns of brain activity in progressive supranuclear palsy and Parkinson's disease, Mov. Disord. 30 (9): 1248-58 (2015)).

Volumetric MRI is a technique where MRI scanners determine volume differences in regional brain volume. This may be done, for example, by contrasting different disorders, or by determining differences in volume of a brain region in a patient over time. Volumetric MRI may be employed to determine disease progression or improvement in neurodegenerative disorders like PSP. The technique is well-known to those having ordinary skill in the art. (Messina D, et al., Patterns of brain atrophy in Parkinson's disease, progressive supranuclear palsy and multiple system atrophy, Parkinsonism and Related Disorders, 17 (3): 172-76 (2011)). Examples of cerebral regions which may be measured include, but are not limited to, intracranial volume, cerebral cortex, cerebellar cortex, thalamus, caudate, putamen, pallidum, hippocampus, amygdala, lateral ventricles, third ventricle, fourth ventricle, and brain stem.

h. Neurogenesis

The invention also contemplates treating or improving neurogenesis in a subject with declining or impaired neurogenesis, which may manifest itself, for example, through reduced cognitive or motor function, or through association with neuroinflammation. An embodiment of the invention includes administering, by way of example and not limitation, a blood plasma, a plasma fraction, or a PPF to the subject with reduced or impaired neurogenesis using a Pulsed Dosing treatment regimen.

An embodiment of the invention also contemplates determining the level of neurogenesis before, during, and/or after administration of the blood plasma, plasma fraction, or PPF. Noninvasive techniques for evaluating neurogenesis have been reported. (Tamura Y. et al., J. Neurosci. (2016) 36 (31): 8123-31). Positron emission tomography (PET) used with the tracer, [18F]FLT, in combinations with the BBB transporter inhibitor probenecid, allows for accumulation of the tracer in neurogenic regions of the brain. Such imaging allows for an evaluation of neurogenesis in patients being treated for neurodegenerative disease.

i. Neuroinflammation

The invention also contemplates treating or improving neuroinflammation in a subject with heightened neuroinflammation, which may manifest itself, for example, through reduced cognitive or motor function, or through association with reduced neurogenesis or neurodegeneration. An embodiment of the invention includes administering, by way of example and not limitation, a blood plasma, a plasma fraction, or a PPF to the subject with neuroinflammation using a Pulsed Dosing treatment regimen.

An embodiment of the invention also contemplates determining the level of neuroinflammation before, during, and/or after administration of the blood plasma, plasma fraction, or PPF. Noninvasive techniques for evaluating neuroinflammation have been reported such as TSPO Positron Emission Tomography (TSPO PET) using ¹¹C-PK11195 and other such tracers. (See Vivash L, et al., J. Nucl. Med. 2016, 57:165-68; and Janssen B, et al., Biochim. et Biophys. Acta, 2016, 425-41, herein incorporated by reference). Invasive techniques for evaluating neuroinflammation include drawing of cerebrospinal fluid and detecting, for example, expression levels of neuroinflammatory markers or factors such as (but not limited to) prostaglandin E2, cyclooxygenase-2, TNF-alpha, IL-6, IFN-gamma, IL-10, eotaxin, beta-2 microglobulin, VEGF, glial cell line-derived neurotrophic factor, chiotriosidase-1, MMP-9, CXC motif chemokine 13, terminal complement complex, chitinasc-3-like-protein 1, and ostcopontin. (See Vinther-Jensen T, et al., Neruol Neurimmunol Neuroinflamm, 2016, 3 (6): e287; and Mishra et al., J. Neuroinflamm., 2017, 14:251 herein incorporated by reference).

### 13. Reagents, Devices, and Kits

Also provided are reagents, devices, and kits thereof for practicing one or more of the above-described methods. The subject reagents, devices, and kits thereof may vary greatly.

Reagents and devices of interest include those mentioned above with respect to the methods of preparing plasma-comprising blood product for transfusion into a subject in need hereof, for example, anti-coagulants, cryopreservatives, buffers, isotonic solutions, etc.

Kits may also comprise blood collection bags, tubing, needles, centrifugation tubes, and the like. In yet other embodiments, kits as described herein include two or more containers of blood plasma product such as plasma protein fraction, such as three or more, four or more, five or more, including six or more containers of blood plasma product. In some instances, the number of distinct containers of blood plasma product in the kit may be 9 or more, 12 or more, 15 or more, 18 or more, 21 or more, 24 or more 30 or more, including 36 or more, e.g., 48 or more. Each container may have associated therewith identifying information which includes various data about the blood plasma product contained therein, which identifying information may include one or more of the age of the donor of the blood plasma product, processing details regarding the blood plasma product, e.g., whether the plasma product was processed to remove proteins above an average molecule weight (such as described above), blood type details, etc. In some instances, each container in the kit includes identifying information about the blood plasma contained therein, and the identifying information includes information about the donor age of the blood plasma product, e.g., the identifying information provides confirming age-related data of the blood plasma product donor (where such identifying information may be the age of the donor at the time of harvest). In some instances, each container of the kit contains a blood plasma product from a donor of substantially the same age, i.e., all of the containers include product from donors that are substantially the same, if not the same, age. By substantially the same age is meant that the various donors from which the blood plasma products of the kits are obtained differ in each, in some instances, by 5 years or less, such as 4 years or less, e.g., 3 years or less, including 2 years or less, such as 1 year or less, e.g., 9 months or less, 6 months or less, 3 months or less, including 1 month or less. The identifying information can be present on any convenient component of the container, such as a label, an RFID chip, etc. The identifying information may be human readable, computer readable, etc., as desired. The containers may have any convenient configuration. While the volume of the containers may vary, in some instances the volumes range from 10 ml to 5000 mL, such as 25 mL to 2500 mL, e.g., 50 ml to 1000 mL, including 100 mL to 500 mL. The containers may be rigid or flexible, and may be fabricated from any convenient material, e.g., polymeric materials, including medical grade plastic materials. In some instances, the containers have a bag or pouch configuration. In addition to the containers, such kits may further include administration devices, e.g., as described above. The components of such kits may be provided in any suitable packaging, e.g., a box or analogous structure, configured to hold the containers and other kit components.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

### EXAMPLES

### A. Example 1—Plasma Fractionation Process

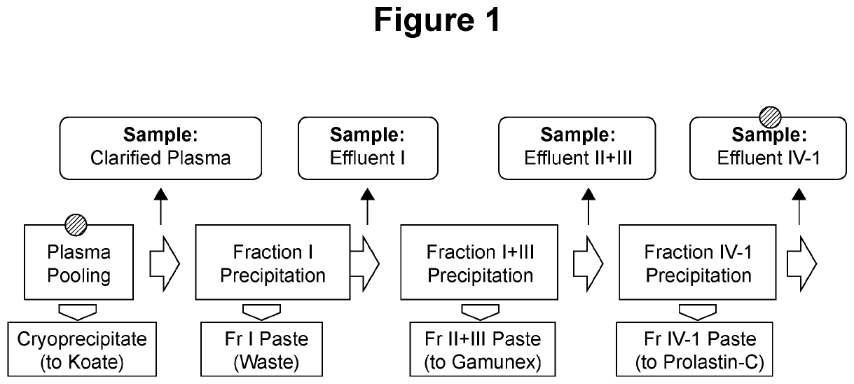

The general process of plasma fractionation is well established. FIG. 1 details the relevant manufacturing process used starting with pooled plasma. Fraction IV-1 paste is an underutilized process intermediate in the manufacturing process, resulting in Alpha-1 Antitrypsin (Prolastin® C) and ATIII products. 40% of IV-1 paste is currently utilized to manufacture the final products, whereas 60% of IV-1 paste is considered a waste. Fraction IV-1 paste can be utilized by dissolving into solution by a variety of methods as described before. (See, Viglio S, et al., Molecules, 25 (17): 4014 (2020); and Chen S X, et al., J Chromatogr A, s02021-9673 (97) (1998)). References to IV-1 paste in the following examples mean a dissolved IV-1 paste solution.

### B. Example 2—Plasma Fraction Activity in Various In Vitro Assays

a. PPF1 and IV-1 Paste and Glucose Utilization in Myotubes

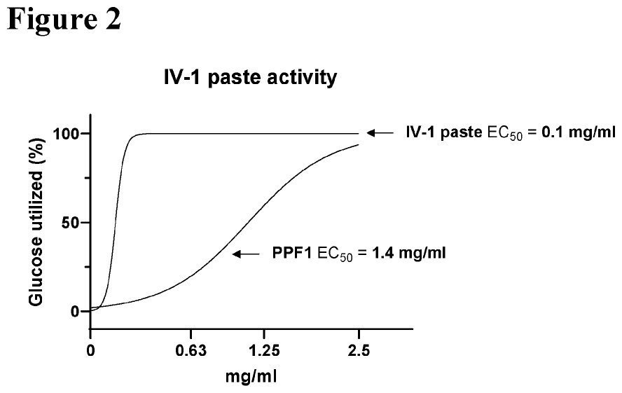

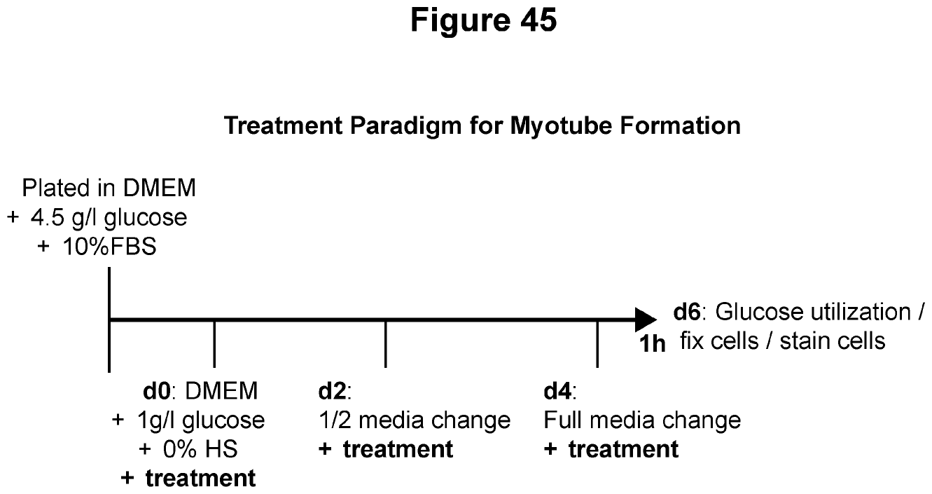

FIG. 2 reports a dose-response relationship between the PPF1, and IV-1 paste. PPF1 was previously identified as an active plasma fraction in this assay (US Patent Application No. US20210128693A1). Myotubes were differentiated to myoblasts as described in FIG. 45. Treatments were added to cells at the following concentrations 2.5, 1.25, 0.63 and 0.315 mg/mL. The two plasma fractions exhibited a dose-response relationship to glucose utilized, reflecting the degree of differentiation of myotubes.

Unexpectedly, IV-1 paste was 14-times more active compared to the positive control PPF1. This suggests that IV-1 paste is an ideal plasma fraction to treat musculoskeletal diseases such as Cachexia and frailty which result in muscle weight loss.

b. Activity of PPF1, HAS1, IV-1 Paste, and IV-4 Paste Across Assays

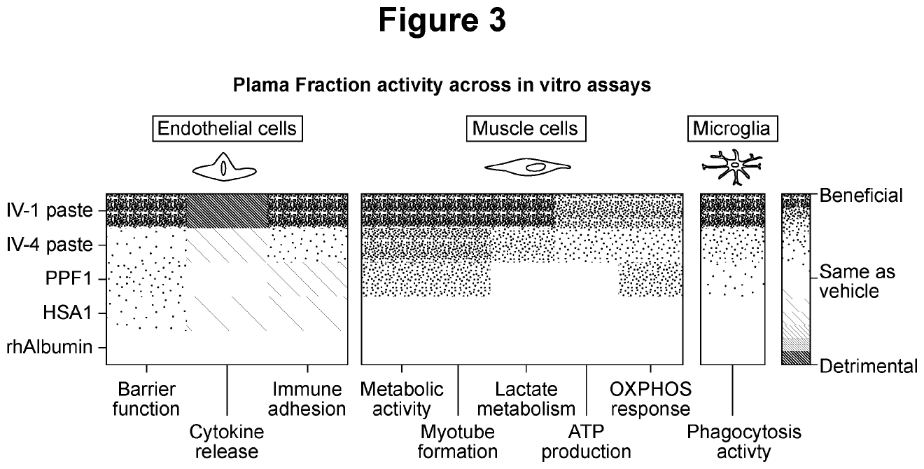

FIG. 3 is a side-by-side comparison of various plasma fractions' activities across in vitro assays. A heat map of various in vitro assays in multiple cell system is shown. The chosen cell systems were endothelial cells (HUVECs), skeletal muscle cells (C2C12), and microglia cells (primary mouse microglia). The activities of the following Plasma fractions were compared to each other: IV-1 paste, IV-4 paste, PPF1, and HAS1 (noted as HSA1 in FIG. 3). As protein loading control recombinant human Albumin (rhAlbumin) was included. Color coding for the heatmap: blue indicates a potential beneficial activity in the corresponding assay and cell type, white indicates the same activity as vehicle, while yellow indicates a potential detrimental activity. (The terms “beneficial activity” and “detrimental activity” are reflective of more common disease indications that may be treated by administration of the plasma fraction. For example, some indications such as infections may find that increased cytokine release is a beneficial activity in treating those indications). All plasma fractions were tested at the same protein concentration. The activity was visualized via prism and normalized to vehicle activity.

Interestingly, IV-1 paste is ~10 times more potent than PPF1 across multiple cellular assays. We found that the fraction IV-1 paste suspension increased barrier function and decreased adhesion molecule surface expression in HUVECs, increased metabolic and regenerative activities in C2C12 cells, and reduced the phagocytotic activity of activated primary microglia. However, the fraction IV-1 paste suspension also induced secretion of IL-8 and IL-6. While this implies a pro-inflammatory response, this data package should be carefully interpreted depending on the indication space selection.

### C. Example 3—Subfractionation of IV-1 Paste and Overall Activity

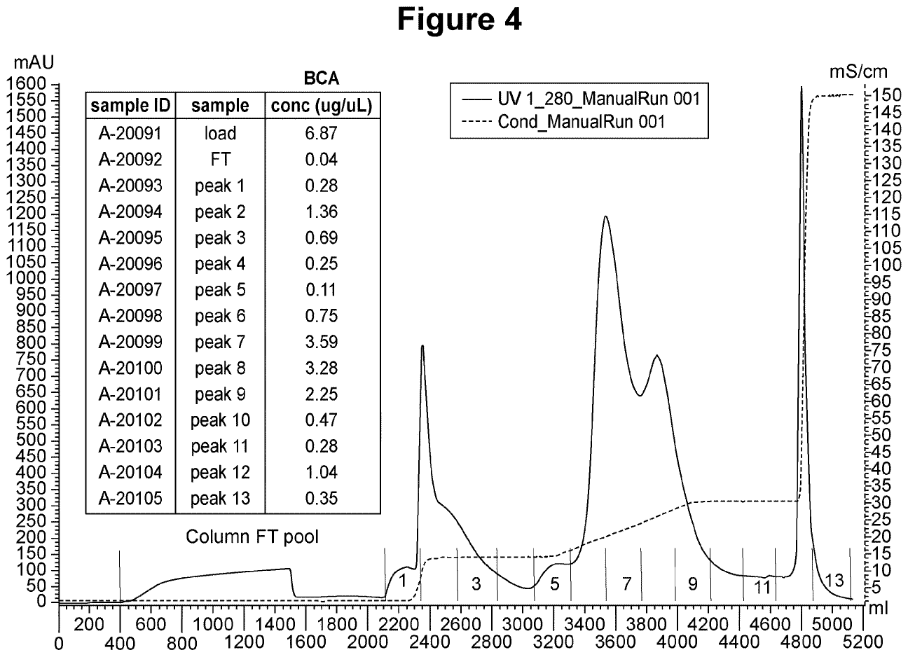

FIG. 4 shows the separation of IV-1 paste in 13 partitions by an anion exchange chromatography column. As an anion exchange matrix, Q-Sepharose was used. Each subfraction was formulated with 0.9% NaCl/10 mM HEPES to prepare test samples. Total protein concentrations from each fraction were measured by a BCA test and listed in FIG. 4. Additionally, the load (material used to load the column) and the FT (flow through) were measured for their total protein concentration.

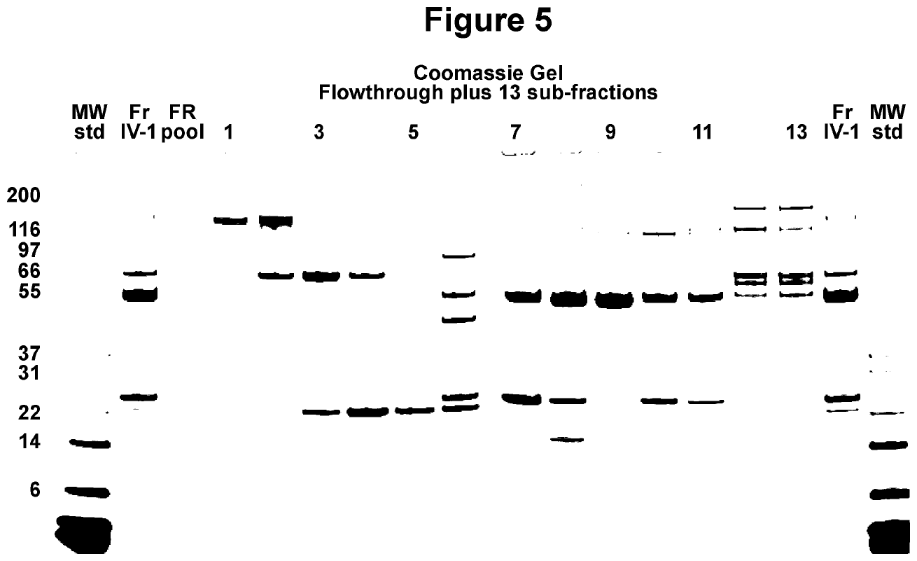

FIG. 5 shows a Coomassie blue staining from the 13 subfractions from IV-1 paste. Each subfraction was loaded with the same protein concentration on the gel. Proteins were first separated on an SDS-PAGE gel. Then the gel was soaked in Coomassie dye to visualize the proteins.

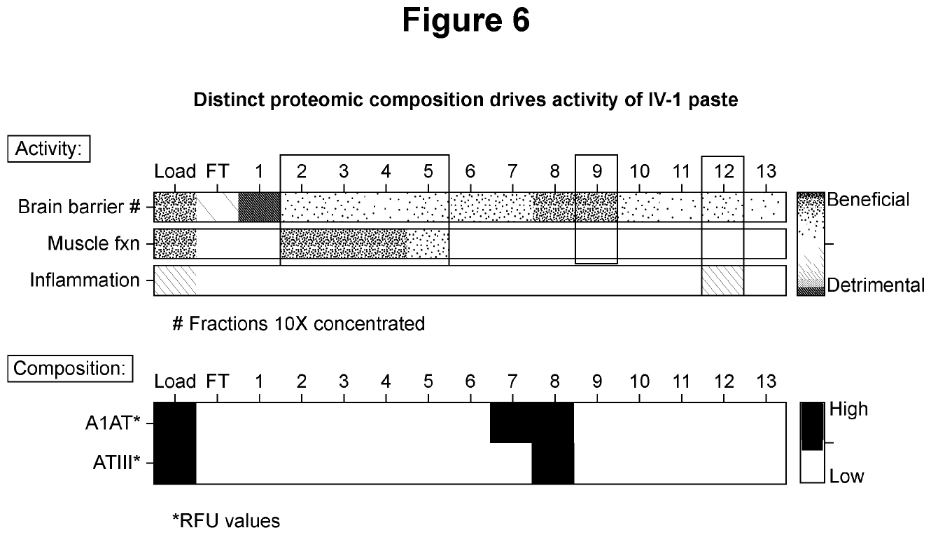

FIG. 6 reports the activity of the load (IV-1 paste), FT, and 13 subfractions in brain barrier, muscle functional, and inflammation assays. Most active fractions are highlighted with a grey rectangle. For the brain barrier assay the fractions were concentrated 10×, whereas the fractions were tested at 1× in the muscle functional and inflammation assays. It was observed that: subfractions 8 and 9 of the chromatography enhanced barrier function; subfraction 12 promoted secretion of IL-6 and IL-8; and subfractions 2 through 5 enhanced muscle activity to more significant degrees relative to their respective other subfractions. Color coding for the heatmap: blue indicates beneficial activity in the corresponding subfraction, white indicates no activity of the subfraction, while yellow indicates detrimental activity. Each subfraction was used at 10% without adjusting the protein concentration. The activity was visualized via prism and normalized to load activity.

RFU values using SomaLogic aptamer technology (SomaLogic Operating Co., Inc., Boulder, CO). were used to compare the abundances of alpha-1-antitrypsin (A1AT) and antithrombin III (ATIII). Color coding for the heatmap is as follows: black indicates high abundancy of the indicated proteins; gray indicates lower abundancy of the proteins relative to black; and white indicates lowest abundancy. Abundancies were visualized via Prism and normalized to load protein abundance. The protein abundancy of the final products from IV-1 paste (i.e., A1AT, and ATIII), did not correlate with the activities of the subfractions. This indicates that these final products (i.e., A1AT, and ATIII), cannot be the drivers of activity in these assays, which was surprising since they are the most notable products obtained from IV-1 paste.

Also surprisingly, the IV-1 paste subfractionation resulted in an uncoupling of this fraction's different biological activities; indicating that multiple therapeutic subfractions could be developed from IV-1 paste, with activities independent of the known bioactives A1AT and ATIII. It was also observed that the increased cytokine release could be separated from observed beneficial effects from IV-1 paste with the subfractionation approach. This was an exciting finding since in some chronic neurodegenerative diseases, such as Alzheimer's and Parkinson's disease, an increase in pro-inflammatory cytokines should be avoided. Based on the observed activity of subfractions 8 and 9, these may be used as therapeutic subfractions to treat or even cure vascular dementia which is caused by conditions that interrupt the flow of blood and oxygen supply to the brain and damaged blood vessels in the brain. Subfractions 2-5 could also be used as therapeutic subfractions to cure musculoskeletal diseases such as Cachexia and frailty which result in loss of muscle mass. Subfraction 12 may be used as therapeutic fraction to treat or cure infectious diseases which are caused by bacteria, viruses and fungi that enter the body and can cause infection.

### D. Example 4—Primary Mouse Microglia Phagocytosis

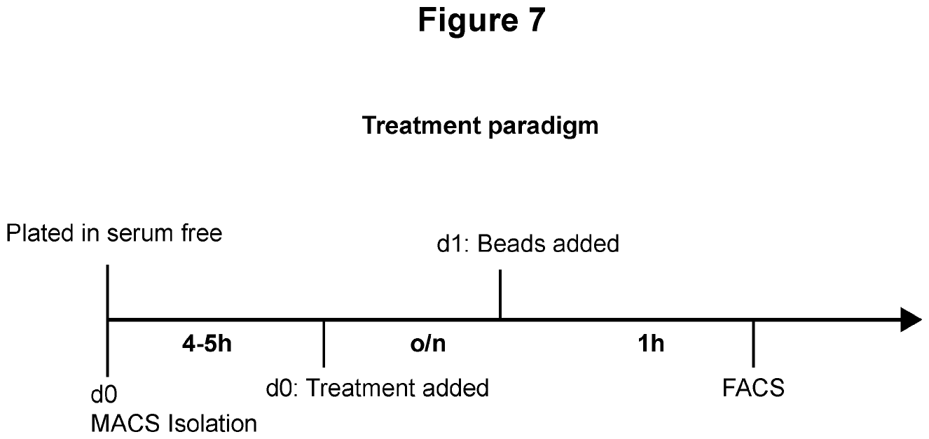

FIG. 7 depicts the treatment paradigm for isolated primary mouse microglia. Primary mouse microglia were isolated from coculture with astrocytes by MACS sorting. Microglia were plated in serum free medium for 4-5 hours. Microglia are a type of immune cell in the central nervous system (CNS) which play a role in maintaining brain homeostasis as well as in responding to injury and infection. The degree of phagocytosis is an indicator of neuroinflammation as a primary role of microglia is to clear debris for tissue repair and remodeling in the brain. Microglia phagocytosis is one of the most affected processes in neurodegenerative diseases. Over-activated microglia phagocytose neurons, neuronal synapses and myelin in conditions like aging, Multiple Sclerosis and Parkinson's disease. This leads to poor signal conductance and changes in brain physiology. Reducing phagocytosis from over-activated microglia is therefore a strategy that can be used in treating these diseases.

Microglia were isolated from post-natal pups and purified by MACS isolation. Various treatments were subsequently added overnight to the microglia. Fluorescent labeled beads were then added to the cells and cultured for an additional 1 hour followed by fixation of the cells. By FACS, the number of cells taking up beads after 1 hour were determined.

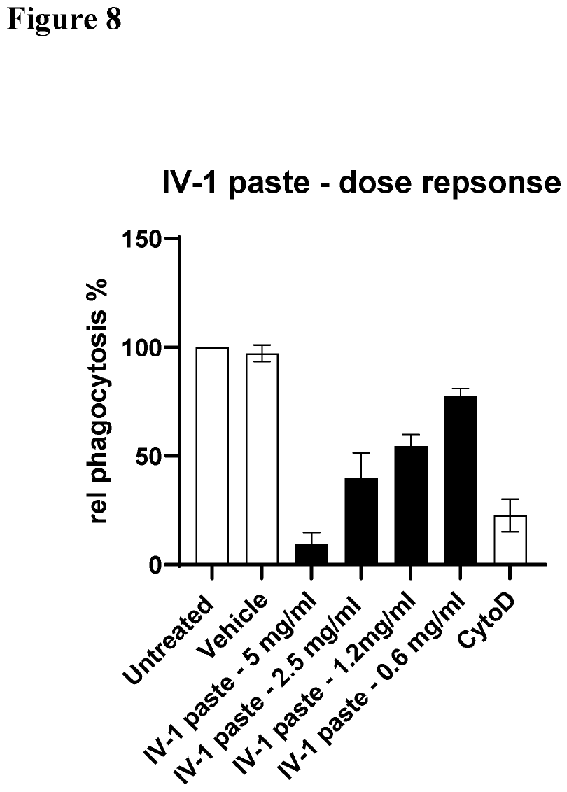

FIG. 8 reports the phagocytotic activity of treated microglia as quantified by FACS normalized to untreated microglia as described in FIG. 7. Microglia were treated with IV-1 paste in a dose responsive manner in a range from 0.6 mg/mL-5 mg/mL. Cyto D was used as a positive control. FIG. 8 shows that IV-1 paste can reduce the phagocytotic activity in microglia in a dose dependent manner. This suggests that in a disease context where microglia are activated, the neuroinflammatory effects of such disease may be attenuated.

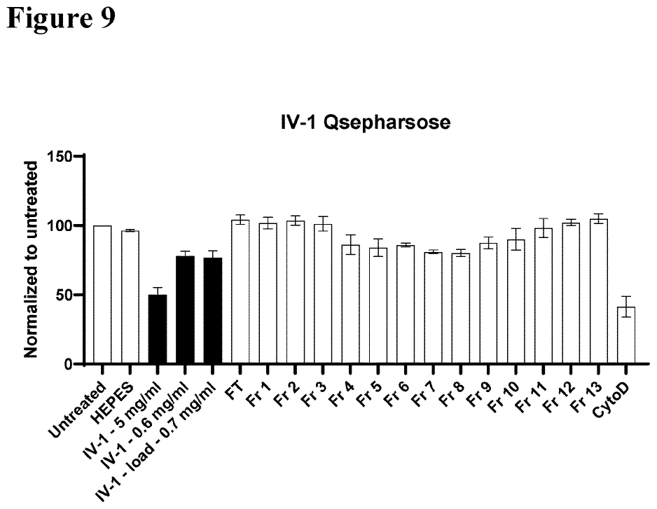

FIG. 9 reports the effects that subfractions of IV-1 paste have on the microglial phagocytosis assay. IV-1 paste was subfractionated into 13 subfractions (Fr 1-Fr 13) using a Q-Sepharose separation column, which is based on anion charge. Subfractions were tested as described in FIG. 7. Based on the separation of the proteins, phagocytic activity was determined. The highest activities were seen in subfractions 7 and 8. (FT=flow through).

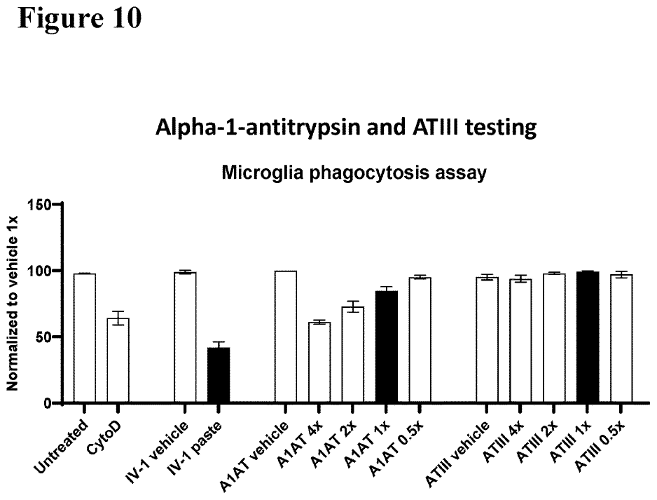

FIG. 10 reports the effects that single purified protein products (A1AT and ATIII) from IV-1 paste have on microglial phagocytotic activity. A1AT demonstrated significant activity in the phagocytic assay. Additionally, a dose response was performed for A1AT. The black bar represents the same amount of protein as is in parent IV-1 fraction (i.e., 1×). ATIII did not significantly impact phagocytotic activity in microglia, but A1AT did demonstrate activity in a dose responsive manner. Surprisingly, as the amount present in IV-1 paste, A1AT only had a slight effect compared to IV-1 paste itself, suggesting that there are additional bioactivities in IV-1 paste not accounted for by A1AT.

### E. Example 5—Adhesion Molecule Expression

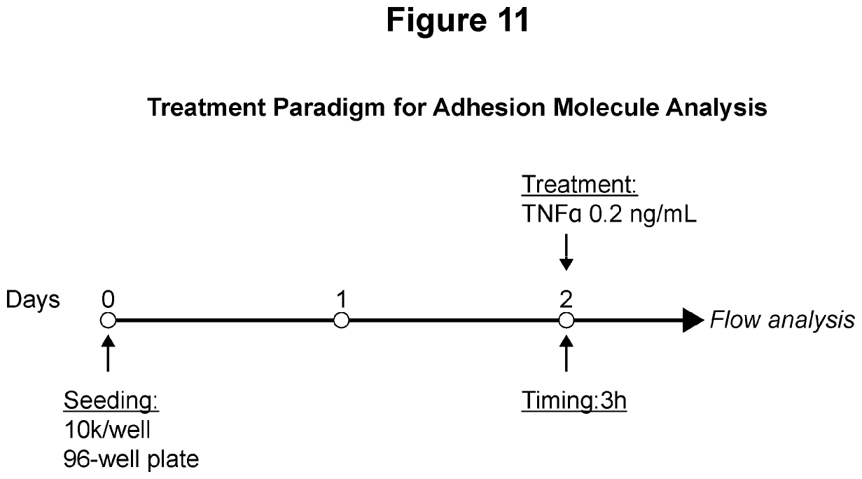

FIG. 11 is a treatment paradigm for analysis of adhesion molecule surface expression in HUVECs. HUVECs (Human Umbilical Vein Endothelial Cells) are primary cells that are essential for maintaining blood vessel integrity. They are a model for studying angiogenesis, vascular permeability, inflammation, and cell adhesion molecule surface expression. The levels of expression of certain adhesion molecules on the cells are related directly to the capability of T-cells and other immune cells to invade tissues, particularly the brain and cause inflammation associated with disease such as peripheral and neuroinflammation. Immune cell recruitment and invasion into the brain has been linked to several neurodegenerative diseases including Parkinson's disease, Alzheimer's disease, multiple sclerosis, neuromyelitis optica and acute disseminated encephalomyelitis.

HUVECs were seeded at 10 k/well in 96 well plates. Cells were cultured for 48 hours and subsequently stressed or not stressed with TNFα at 0.2 ng/ml while concurrently being treated with test proteins or plasma fractions. After 3 hours the amounts of adhesion molecules surface expression were determined via FACS. These included VCAM-1, ICAM-1, and CD62E expression. The quantity of the adhesion molecules was determined as both percent positive cells as well as MFI (mean fluorescence intensity) which quantifies the degree of surface expression of the adhesion molecules.

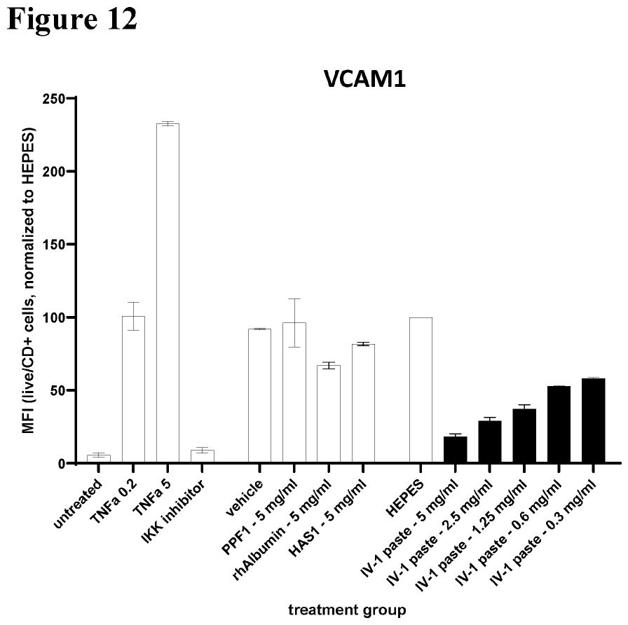

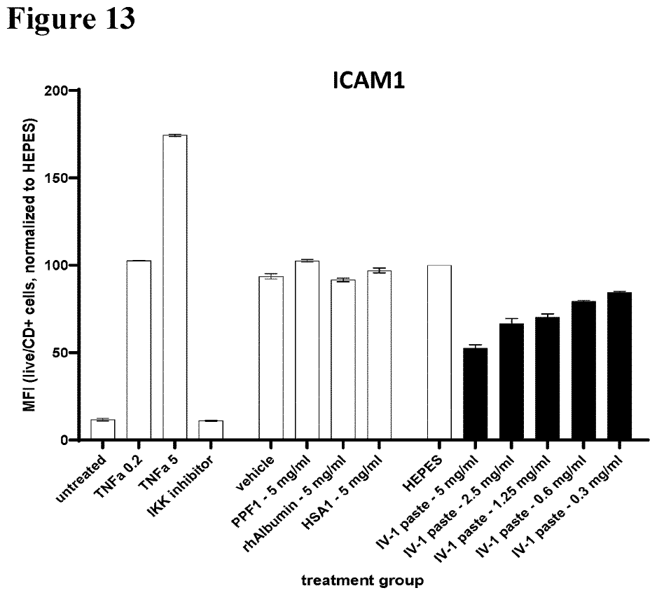

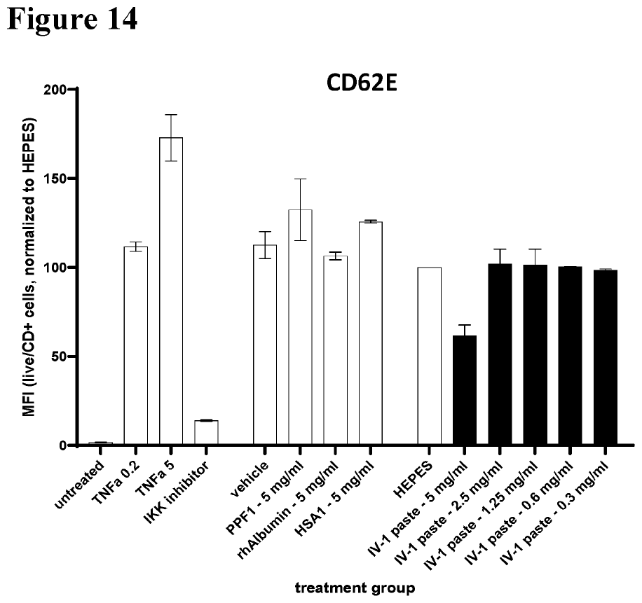

FIG. 12 through FIG. 14 show the results of individual proteins and plasma fractions in the assay described in FIG. 11. A negative control of TNFα (5 ng/ml) and positive control IKK inhibitor were both administered to cells also in the same manner as test proteins/plasma fractions described in FIG. 11. Several plasma fractions were also tested, including PPF1, HAS1, recombinant Albumin (rhAlbumin) as wells as IV-1 paste. All data were normalized to HEPES buffer. IV-1 paste inhibited expression of VCAM-1 adhesion molecule in a dose dependent manner (0.3 mg/mL-5 mg/mL). FIG. 13 reported similar results for ICAM-1 adhesion molecule expression. FIG. 14 reported similar results for CD62E adhesion molecule expression, but the effect was only seen at the highest dose of Fraction IV-1 paste.

Thus, IV-1 paste showed highest efficacy in reducing surface expression of adhesion molecules compared to other tested plasma fractions and proteins. This suggests that in a disease context where surface expression of adhesion molecules leads to invasion of T-cells and other immune cells, the effects of such disease can be reduced by IV-1 paste more effectively than other plasma fractions.

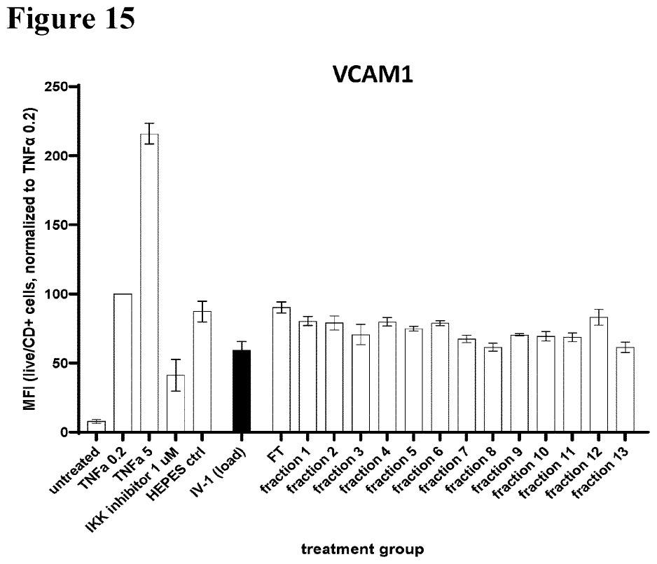

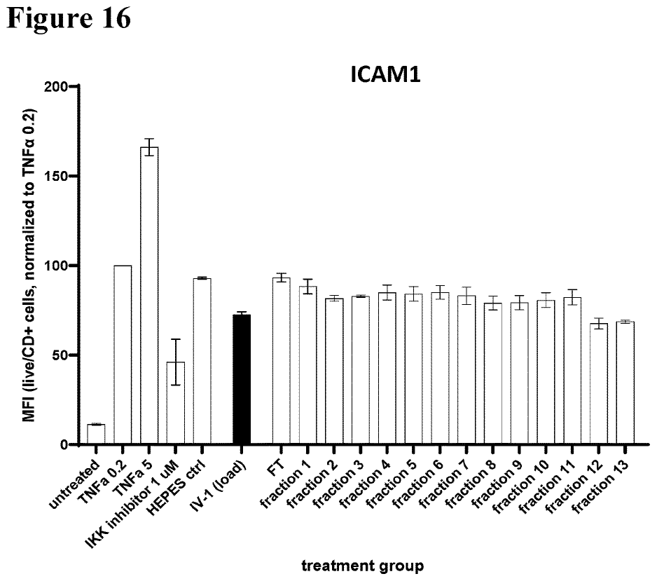

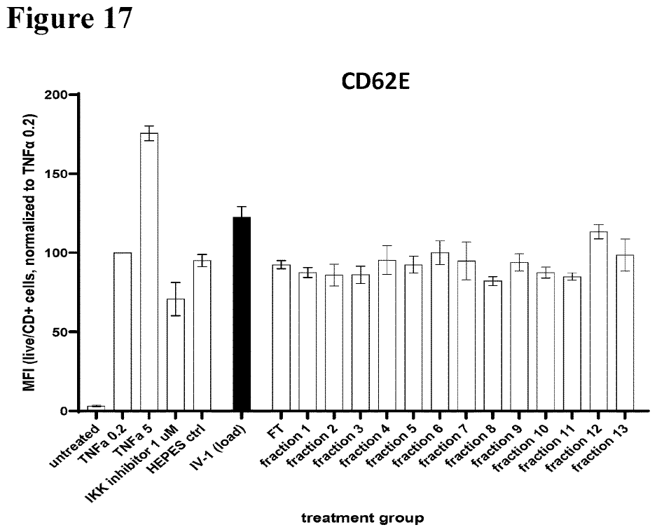

FIG. 15 through FIG. 17 report the effects of the 13 subfractions of fraction IV-1 paste on these adhesion molecules as described in the assay of FIG. 11. No subfractions stood out as showing significantly clear activity in reducing expression of the adhesion molecules VCAM-1, ICAM-1, or CD62E.

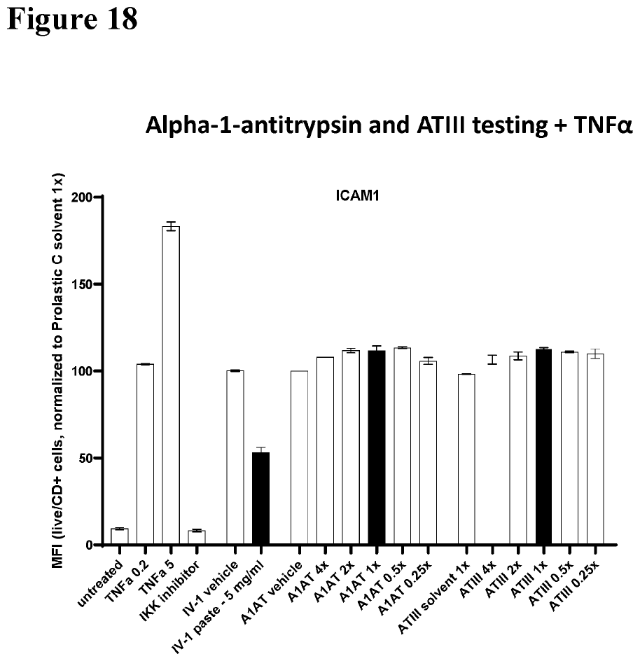

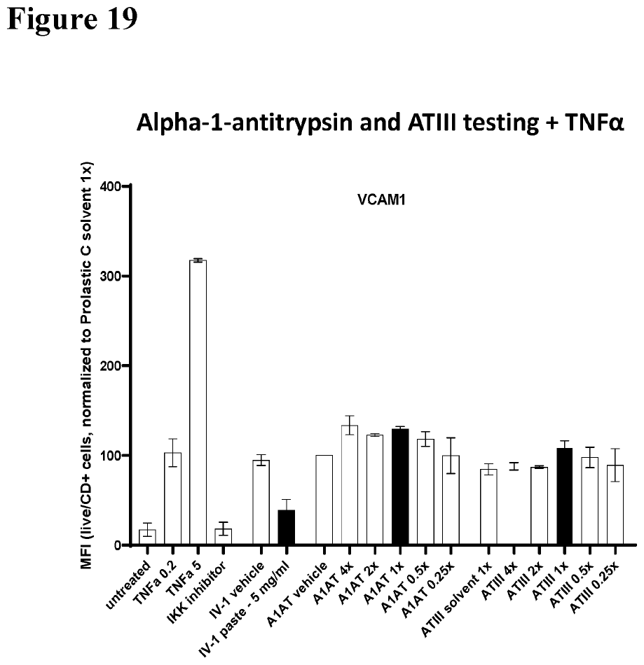

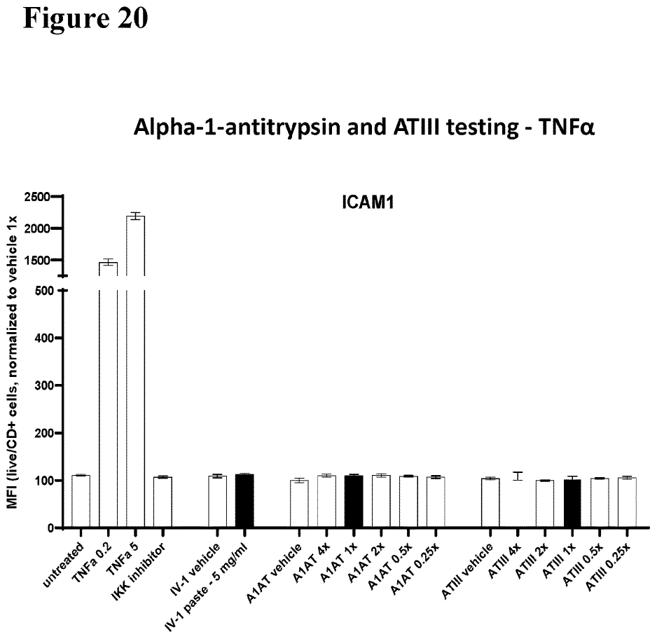

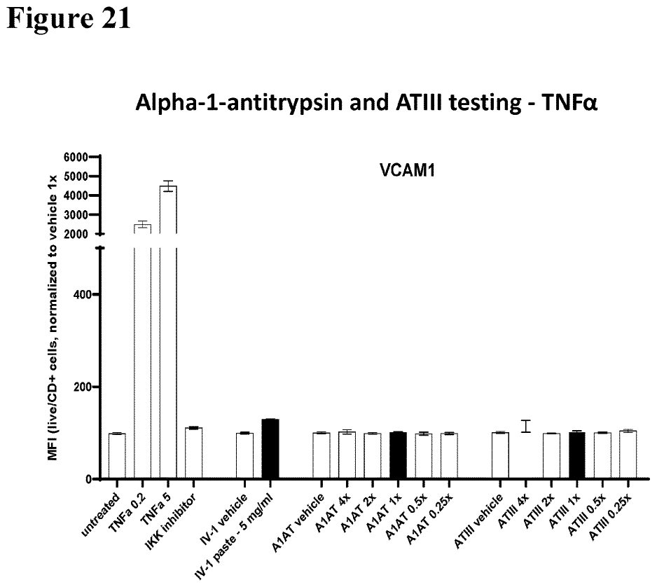

FIG. 18 through FIG. 21 report the effects of single protein products purified from fraction IV-1 paste (A1AT and ATIII) on adhesion molecule surface expression. Neither protein appears to contribute to the activity of IV-1 paste to reduce adhesion molecule surface expression (VCAM-1 and ICAM-1). This was done both in the context of TNFα stress (FIGS. 18 and 19) and without TNFα stress (FIGS. 20 and 21).

### F. Example 6—Barrier Function Assay

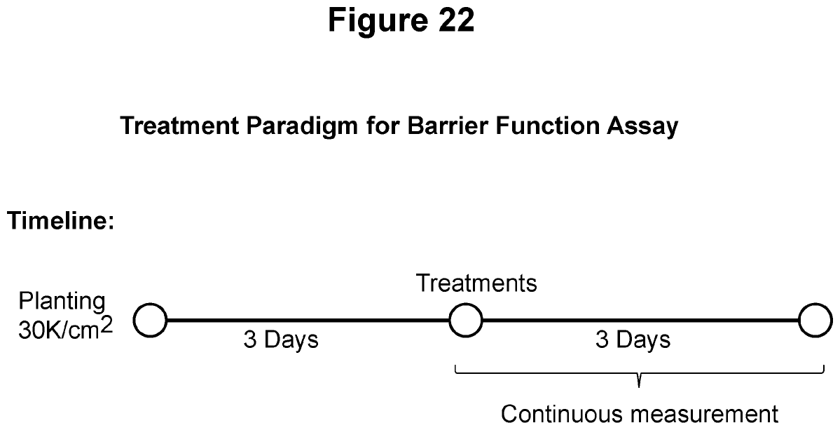

FIG. 22 is a depiction of the treatment paradigm for a Barrier Function Assay. HUVECs were seeded at 30 k/sq cm in CytoZ 96 well plates and cultured for 3 days. The cells were then treated with plasma fractions or controls and barrier function was measured continuously during those 3 days using a Maestro Pro impedance function at 1 kHz. Endothelial barrier function plays a crucial role in maintaining integrity of vasculature and blood brain barrier. Disruption of this barrier can lead to the entry of harmful substances into the surrounding tissue and contribute to disease progression. Multiple sclerosis, stroke, Alzheimer's and Parkinson's disease as well as HIV-associated neurocognitive disorders (HAND) are few examples of CNS disease where endothelial barrier function is disrupted. This disruption facilitates perfusion of inflammatory cells and toxic molecules into brain parenchyma leading to degeneration of neurons. Improving or restoring barrier function would be beneficial to treat or stop progression of such diseases. Additionally, peripheral indications have been associated with loss of barrier function including, for example, Acute respiratory distress syndrome (ARDS), sepsis, inflammatory bowel disease (IBD), atherosclerosis, diabetes mellitus, cancer, and allergic reactions.

The barrier function assay is useful in determining the integrity and function of the endothelial cell barrier that HUVEC cells form, which is critical in regulating passage of molecules and cells between the bloodstream and other tissues such as, for example, the brain. In the brain, the assay reflects the function and integrity of the blood brain barrier.

Transendothelial Electrical Resistance (TEER) is an assay that can be performed to measure resistance to the passage of an electric current across the HUVECs. Resistance to 1 kHz frequency is directly proportional to a healthy, more intact tight barrier for endothelial cells.

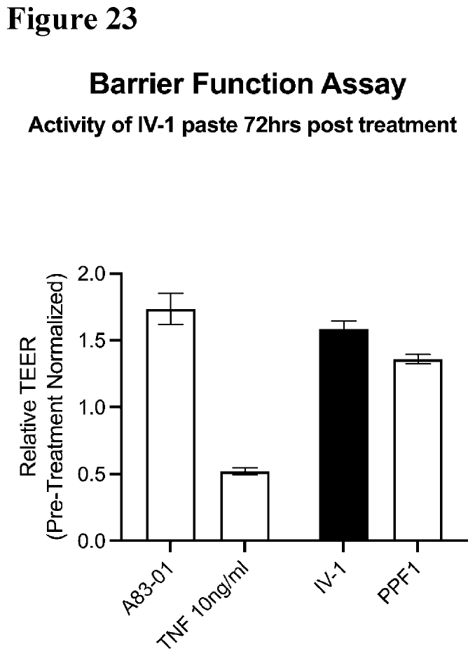

FIG. 23 reports the effects of Fraction IV-1 paste and PPF1 on relative TEER normalized to pre-treatment. A83-01, a potent TGFbeta inhibitor, is a positive control that increases barrier function. TNFα is a negative control that is known to decrease barrier function. Both Fraction IV-1 paste and PPF1 increased barrier function 72 hours post treatment, but IV-1 paste was more effective than PPF1. The TEER assay reflects how closely the endothelial cells are with one another which results in less leakiness of proteins through endothelium, which is the most important component of the blood brain barrier. Increased leakiness and loss of integrity of the blood brain barrier has been implicated in neurodegenerative disease resulting in, among other things, cognitive impairment.

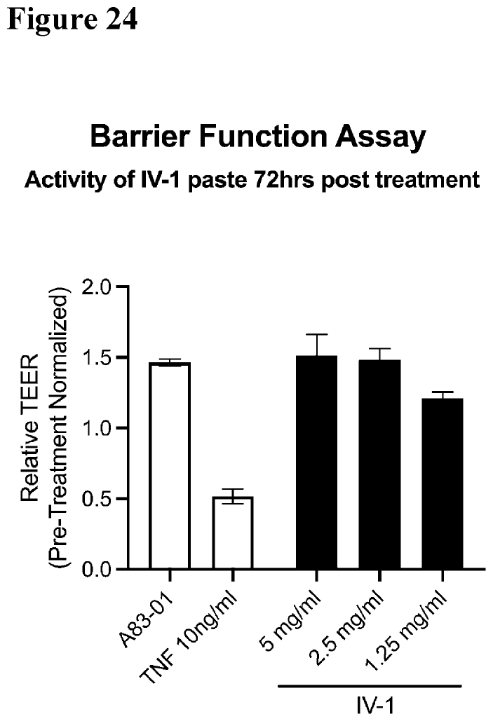

FIG. 24 is a dose-response study of Fraction IV-1 paste 72 hours post treatment using the TEER assay. A83-01 is a positive control that increases barrier function. TNFα is a negative control that is known to decrease barrier function. This study shows that Fraction IV-1 paste can improve barrier function in a dose-responsive manner.

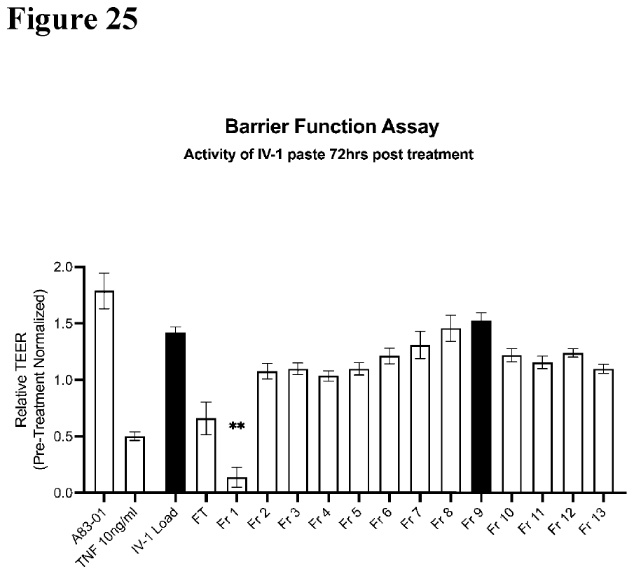

FIG. 25 shows the results of the TEER assay with cells that were treated with Fraction IV-1 paste, flow-through (FT), and the subfractions 1 through 13 from Fraction IV-1 paste. A83-01 is a positive control that increases barrier function. TNFα is a negative control that is known to decrease barrier function. The 13 subfractions were tested as described in FIG. 22. However, these subfractions were concentrated 10-fold. Fraction 9 showed the highest activity in the Barrier Function Assay. Fraction 8 also showed significantly increased activity in this assay.

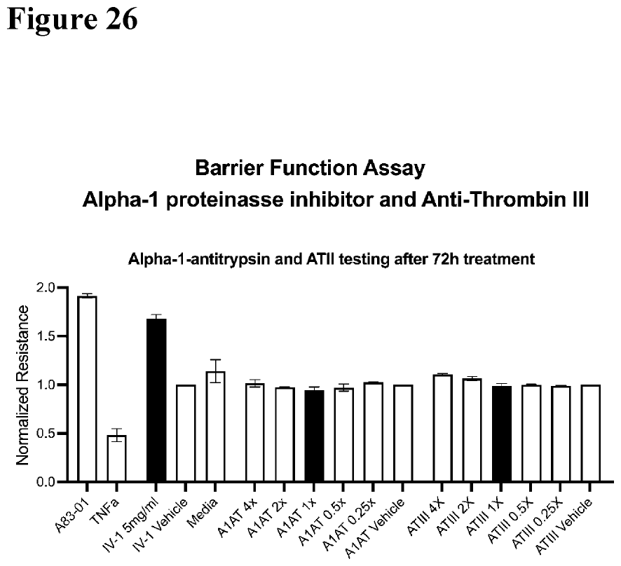

FIG. 26 shows the results of the TEER assay with cells that were treated with Fraction IV-1 paste, the single protein alpha-1 antitrypsin (A1AT) at different concentrations, and the single protein antithrombin III (ATIII) at different concentrations. A83-01 is a positive control that increases barrier function. TNFα is a negative control that is known to decrease barrier function. The single proteins A1AT and ATIII did not show significant activity remarkably revealing that other drivers must be in the Fraction IV-1 paste and certain of the 13 Fraction IV-1 paste subfractions.

### G. Example 7—Endothelial Cell Proliferation Assay

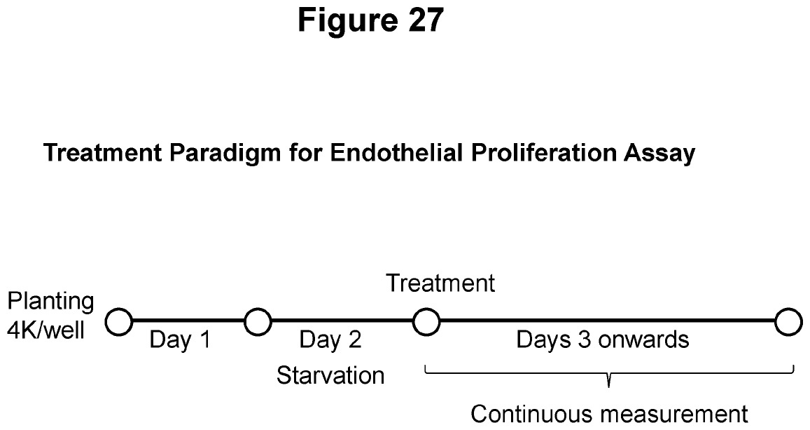

FIG. 27 shows a treatment paradigm for an endothelial cell proliferation assay. HUVECs were seeded at 4K/well in a CytoZ 96 well plate. Cells were cultured for one day and then serum starved for an additional 24 hours in low serum media (0.1% FBS). Cells were then treated with plasma fractions or subfractions for 3 days and continually measured for Maestro Pro impedance function. A resistance of 41.5 kHz was used to determine the degree of confluence which reflects endothelial cell proliferation. Endothelial cells are main constituents of cardiovascular system which allows exchange of essential cells and molecules between bloodstream and the target tissue. Loss of endothelium is observed in many cardiometabolic diseases exacerbating the patient outcome. Atherosclerosis, type 2 diabetes and septic shock are a few examples where endothelial cell loss leads to multi organ (e.g. heart, kidney, eyes) failures. Loss of endothelial cells are also a common feature of Pulmonary Arterial Hypertension and Inflammatory Bowel Diseases (IBD) contributing to dysfunction of lungs and gut. Promoting endothelial cell regeneration may hold promise in treating or managing such conditions where endothelial cell loss is a major part of the disease mechanism,

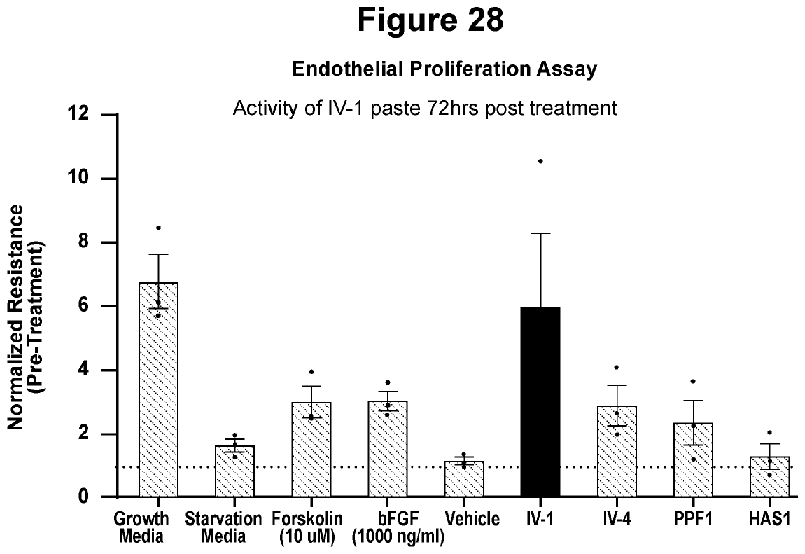

FIG. 28 shows the results of the HUVEC endothelial cell proliferation assay as described in FIG. 27. A positive control of growth media was used. Forskolin and bFGF were also used as positive controls. Fraction IV-1 paste, Fraction IV-4 paste, PPF1, and HAS1 were tested side by side. Fraction IV-1 paste showed the highest activity compared to the other fractions after 72 hours.

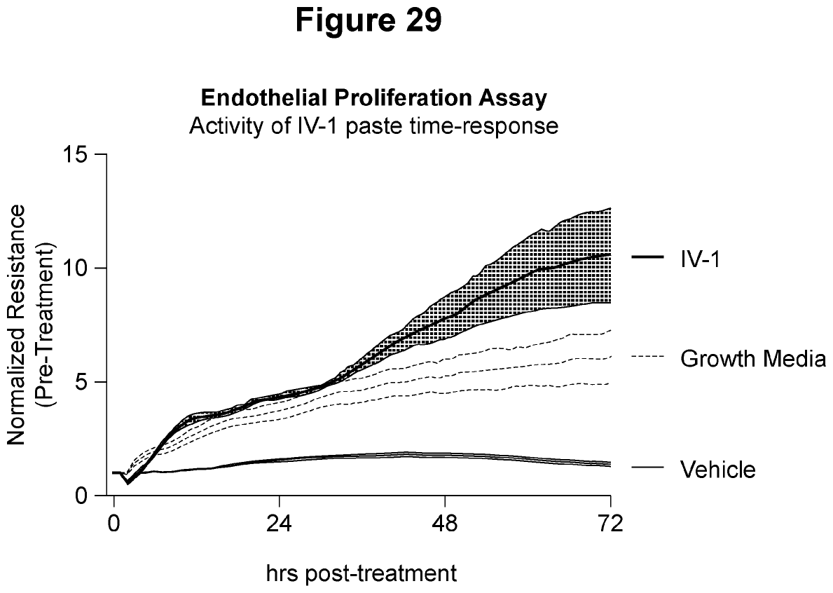

FIG. 29 shows the result of a time response experiment for the HUVEC endothelial cell proliferation assay. Growth media positive control, Fraction IV-1 paste, and vehicle were administered for three days, with measurements taken over time. This shows that Fraction IV-1 paste acts in a time-dependent manner.

### H. Example 8—Cytokine Release Assay

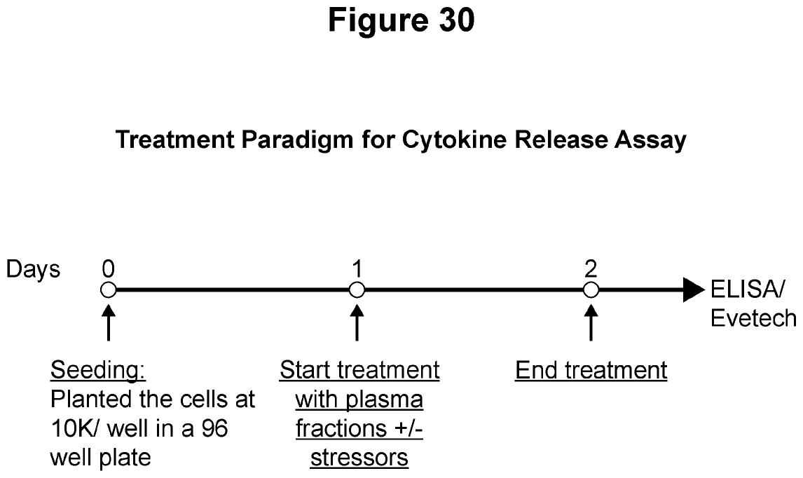

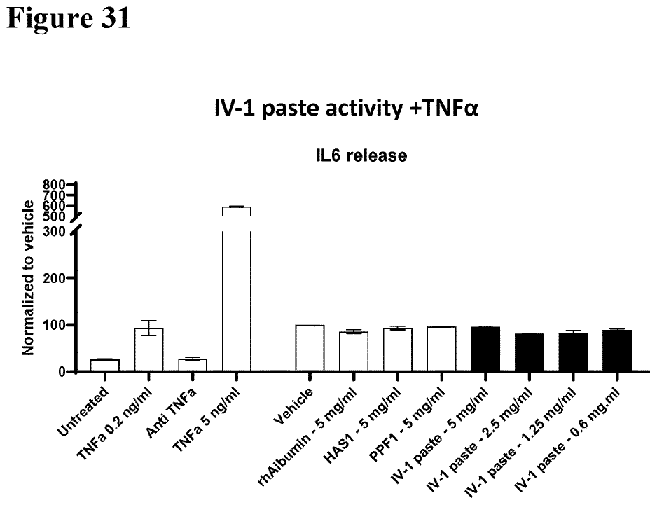

FIG. 30 is a depiction of the treatment paradigm for a cytokine release assay. HUVECs were seeded at 10 k/well in 96 well plate. After 24 hours the cells were treated with or without TNFα stressor at 0.2 ng/mL while treated concurrently with plasma fractions. After an additional 24 hours the cytokine release from the cells was measured via ELISA for IL-6 and IL-8. Triggering an inflammatory response could be beneficial in conditions like injury and infection, where the body needs to activate the immune system. Increase in pro-inflammatory cytokine release is a beneficial biological response against infectious agents, aiming to recruit immune cells and eliminate the pathogen. Such infectious agents could be bacteria, viruses, or fungus infection. Common infectious diseases include sepsis, pneumonia, meningitis, tuberculosis (TB), COVID-19, influenza, HIV, human papillomavirus (HPV), and herpes. FIG. 31 shows the results for IL-6 release using multiple plasma fractions (HAS1, PPF1, and IV-1 paste) or recombinant human albumin (rhAlbumin) under TNFα stress. The plasma fractions and rhAlbumin were administered as described in FIG. 30. None of the tested fractions or rhAlbumin stimulated IL-6 release when stressed with TNFα.

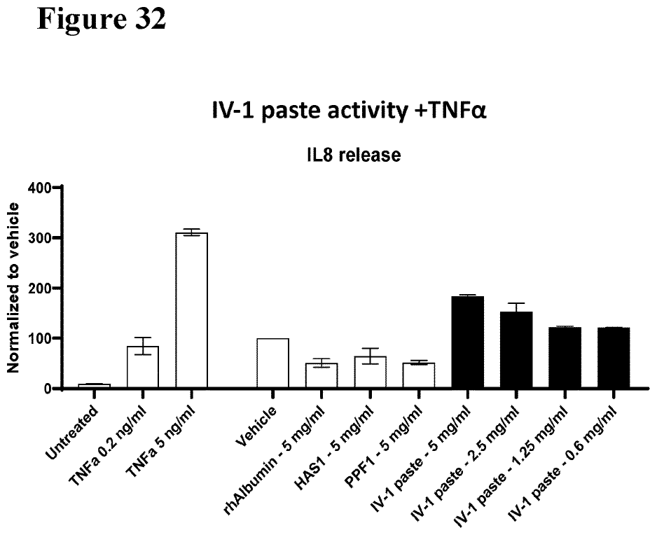

FIG. 32 shows the results for IL-8 release using multiple plasma fractions (HAS1, PPF1, and IV-1 paste) or recombinant human albumin (rhAlbumin) under TNFα stress. There was an increase in IL-8 release upon IV-1 paste treatment in a dose-dependent manner compared to HAS1, PPF1 and rhAlbumin.

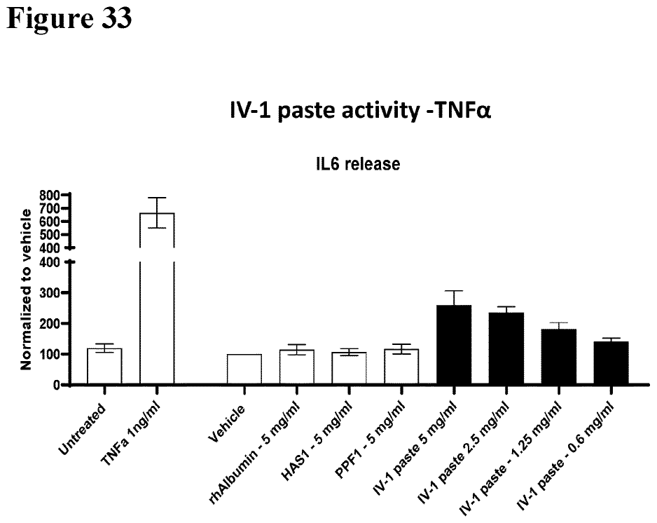

FIG. 33 shows the results for IL-6 release using multiple plasma fractions (HAS1, PPF1, and IV-1 paste) or recombinant human albumin (rhAlbumin) under no TNFα stress. The plasma fractions and rhAlbumin were administered as described in FIG. 30. Only IV-1 paste resulted in an increase in IL-6 release and did so in a dose-dependent manner.

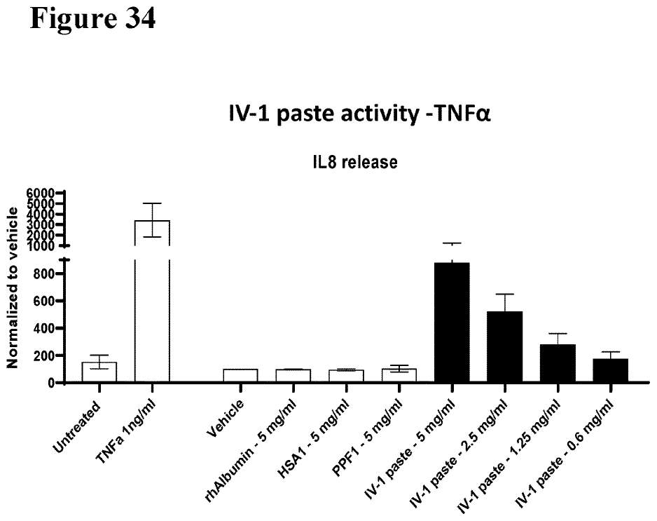

FIG. 34 shows the results for IL-8 release using multiple plasma fractions (HAS1, PPF1, and IV-1 paste) or recombinant human albumin (rhAlbumin) under no TNFα stress. The plasma fractions and rhAlbumin were administered as described in FIG. 30. Only IV-1 paste resulted in an increase in IL-8 release and did so in a dose-dependent manner.

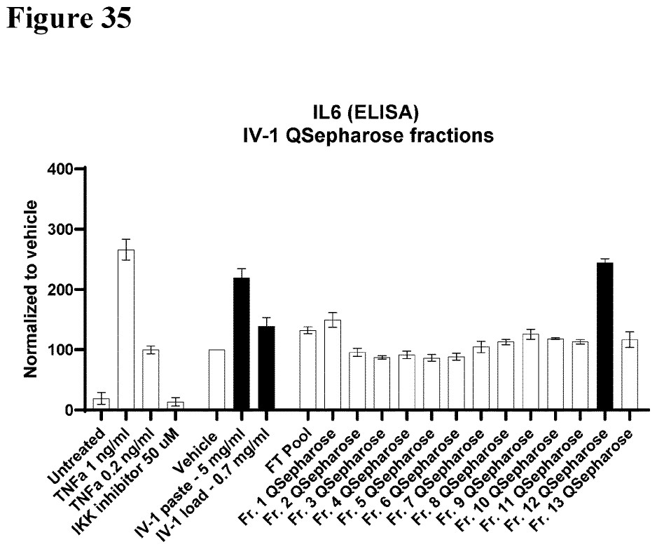

FIG. 35 Thirteen (13) Q-Sepharose subfractions subfractionated from fraction IV-1 paste were tested in the cytokine release assay with TNFα stressor alongside fraction IV-1 paste and the FT. Subfraction 12 increased IL-6 release significantly, but none of the other fractions did. From this result, it appears that the driver of the increase in IL-6 identified in fraction IV-1 paste appears to be in subfraction 12. This shows that a potential undesirable effect of fraction IV-1 paste (i.e., inflammation) can be separated from its more desirable characteristics.

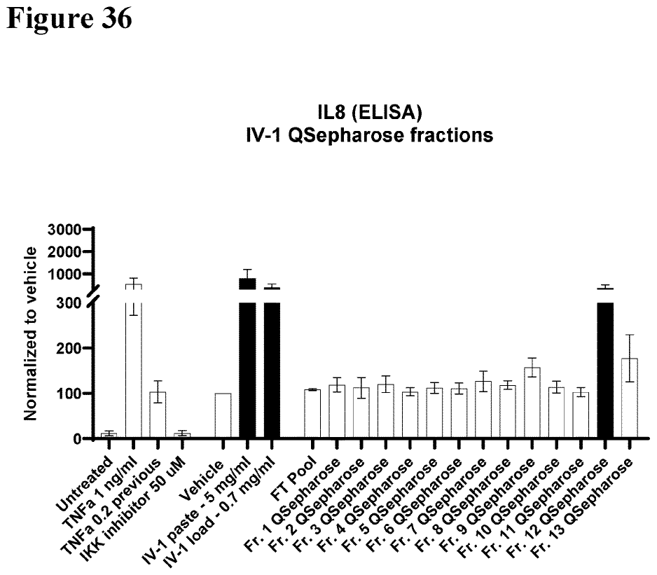

FIG. 36 reports IL-8 release results from testing thirteen (13) Q-Sepharose subfractions in the cytokine release assay described in FIG. 30. Subfractions subfractionated from fraction IV-1 paste were tested in the cytokine release assay with TNFα stressor alongside fraction IV-1 paste and FT. Subfraction 12 increased IL-8 release significantly, but none of the other fractions did. From this result, it appears that the driver of the increase in IL-6 identified in fraction IV-1 paste appears to be in subfraction 12. This shows that a potential undesirable effect of fraction IV-1 paste (i.e., inflammation) can be separated from its more desirable characteristics.

The fact that the undesirable pro-inflammatory activities could be so dramatically and cleanly fractionated from the other subfractions was unexpected. Elimination of these potentially undesirable characteristics found in Subfraction 12 allows the opportunity to use subfractions with positive activities in treating multiple disease areas.

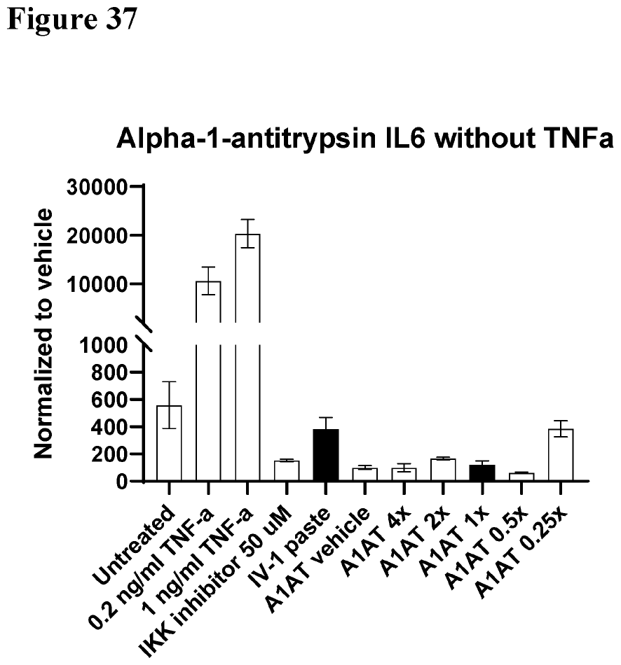

FIG. 37 reports the results of testing the single purified protein product alpha-1 antitrypsin (A1AT) derived from IV-1 paste for cytokine release. IL-6 release without TNFα stressor demonstrated that there was no increased release of IL-6 in the cells by administration of A1AT.

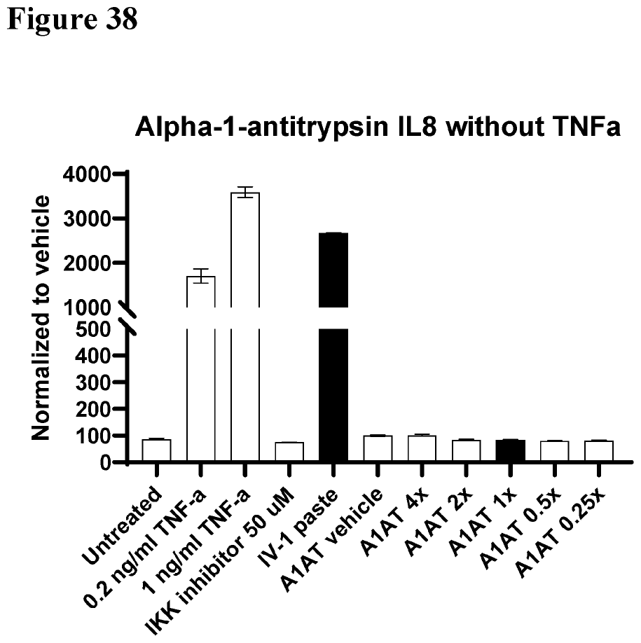

FIG. 38 reports the results of testing the single purified protein product alpha-1 antitrypsin (A1AT) derived from IV-1 paste for cytokine release. IL-8 release without TNFα stressor demonstrated that there was no increased release of IL-8 in the cells by administration of A1AT.

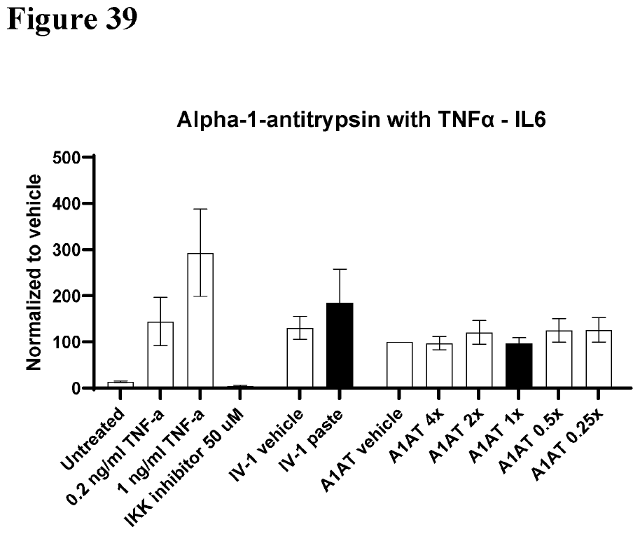

FIG. 39 reports the results of testing the single purified protein product alpha-1 antitrypsin (A1AT) derived from IV-1 paste for cytokine release. IL-6 release with TNFα stressor demonstrated that there was no increased release of IL-6 in the cells by administration of A1AT.

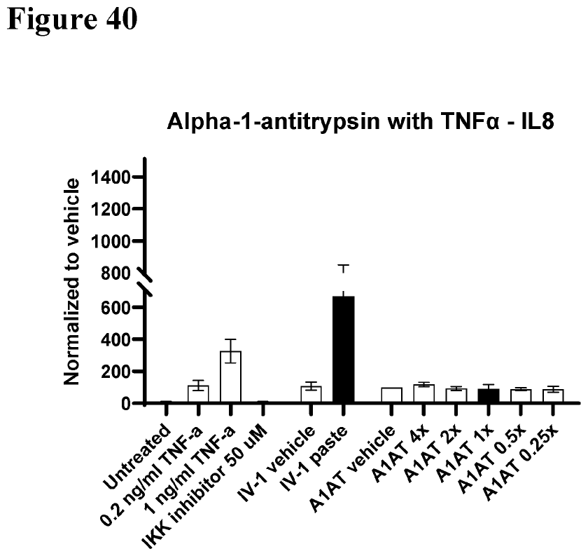

FIG. 40 reports the results of testing the single purified protein product alpha-1 antitrypsin (A1AT) derived from IV-1 paste for cytokine release. IL-8 release with TNFα stressor demonstrated that there was no increased release of IL-8 in the cells by administration of A1AT

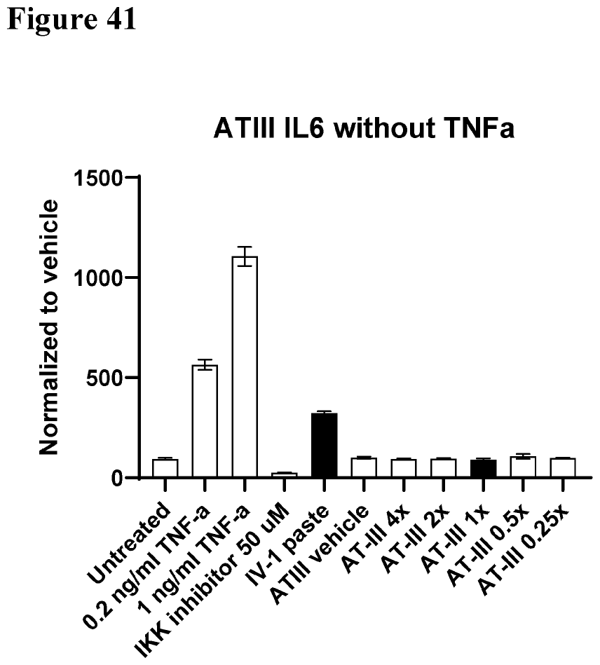

FIG. 41 reports the results of testing the single purified protein product antithrombin III (ATIII) derived from IV-1 paste for cytokine release. IL-6 release without TNFα stressor demonstrated that there was no increased release of IL-6 in the cells by administration of ATIII.

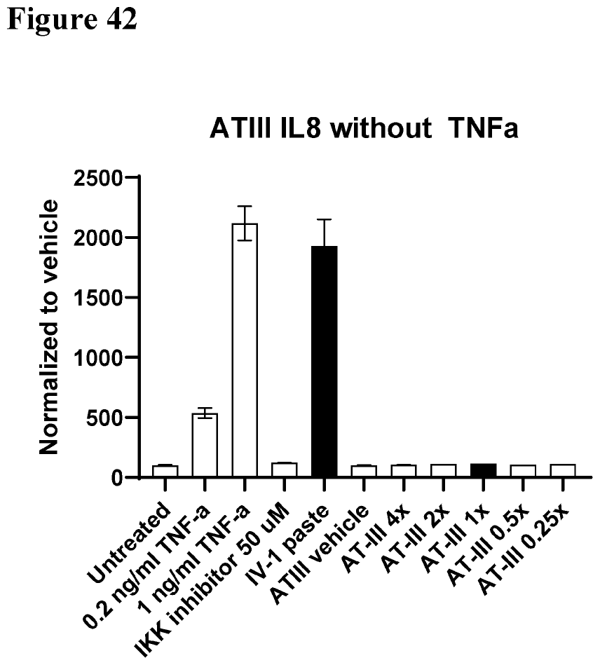

FIG. 42 reports the results of testing the single purified protein product antithrombin III (ATIII) derived from IV-1 paste for cytokine release. IL-8 release without TNFα stressor demonstrated that there was no increased release of IL-8 in the cells by administration of ATIII.

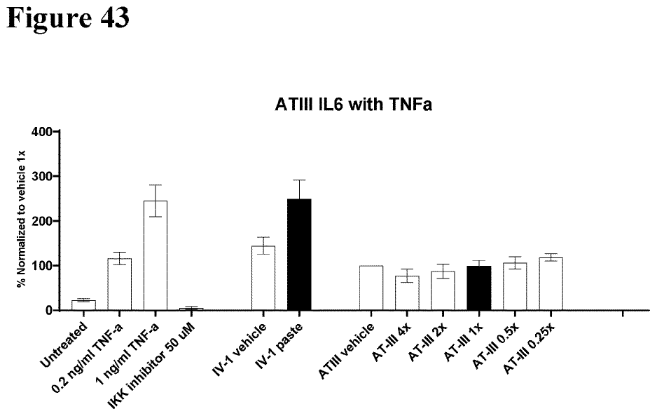

FIG. 43 reports the results of testing the single purified protein product antithrombin III (ATIII) derived from IV-1 paste for cytokine release. IL-6 release with TNFα stressor demonstrated that there was no increased release of IL-6 in the cells by administration of ATIII.

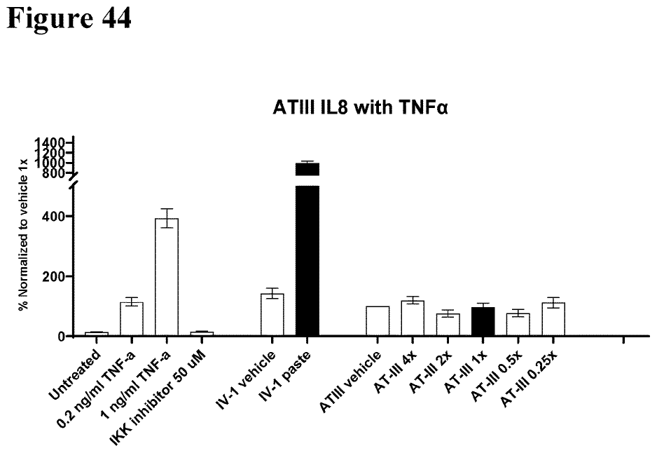

FIG. 44 reports the results of testing the single purified protein product antithrombin III (ATIII) derived from IV-1 paste for cytokine release. IL-8 release with TNFα stressor demonstrated that there was no increased release of IL-8 in the cells by administration of ATIII.

### I. Example 9—C2C12 Myotube Formation Assay

FIG. 45 is a depiction of the treatment paradigm for a C2C12 myoblast differentiation assay. C2C12 myoblasts were seeded at 8 k/well in a 96 well plate in DMEM plus 4.5 g/L glucose, and 10% fetal bovine serum (FBS). After 2 days they were differentiated to myotubes by changing the media to 1 g/L glucose and 0% horse serum. Controls and treatments were added during this differentiation. Every other day, a media change was made as well as the concurrent addition of the different treatments as outlined in the timeline. After 6 days, the amount of glucose used by the cells was determined by quantitative colorimetric assay. The amount of glucose used reflects how well the myoblasts differentiated into myotubes, the latter which are more metabolically active. Well differentiated and metabolically active myotubes are hallmarks of healthy skeletal muscle. Increase in myotube formation indicates that a therapeutic plasma fraction could be used to treat musculoskeletal diseases which result in loss of muscle mass, such as, Cachexia, frailty, Charcot-Marie-Tooth disease, congenital muscular dystrophy, and Congenital fiber type disproportion.

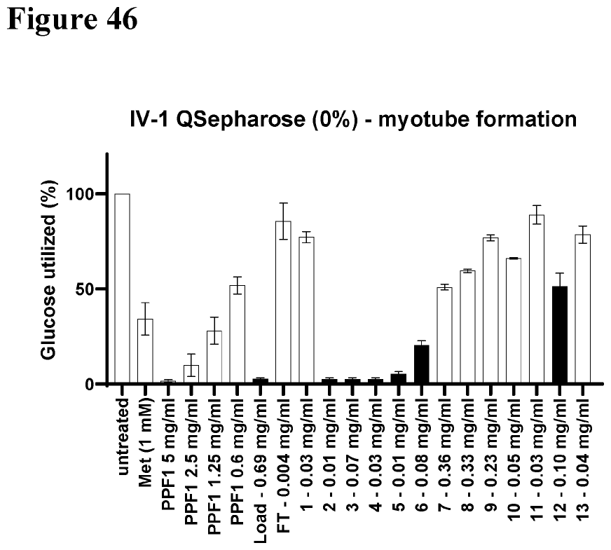

FIG. 46 shows the results of the effects of the 13 subfractions of fraction IV-1 paste on the myotube formation assay described in FIG. 45. Higher degree of myotube differentiation correlates with higher metabolic activity and with lower values on the y-axis. Subfractions 2 through 6 showed high activity in promoting myotube formation in C2C12 cells. This is unexpected and surprising because these fractions show activity when they were also different than those subfractions in HUVEC and primary microglia cells that displayed activity. (e.g., cytokine release, and microglia phagocytosis assay).

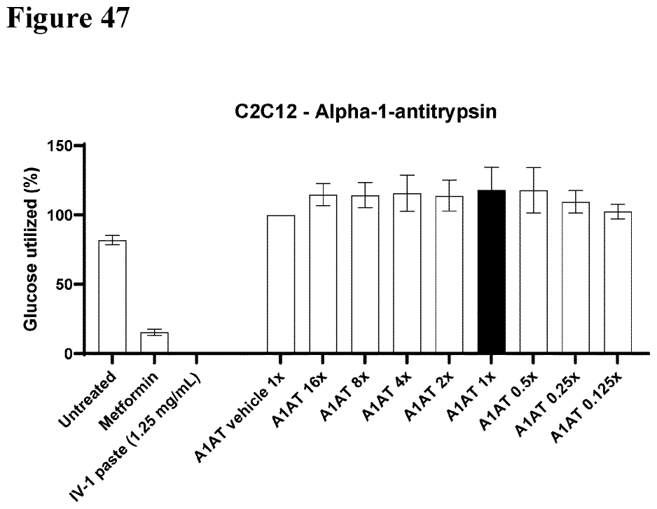

FIG. 47 reports the effects of the single protein product alpha-1 antitrypsin (A1AT) which is purified from fraction IV-1 paste on the myotube formation assay described in FIG. 45. All data were normalized to A1AT vehicle. A1AT displayed no significant activity.

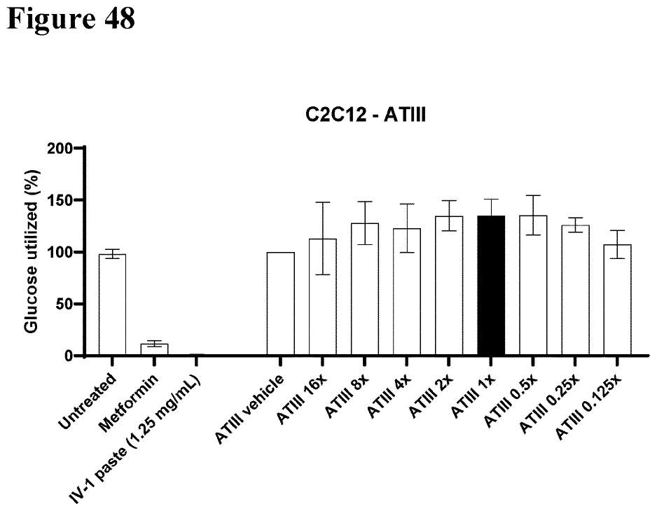

FIG. 48 reports the effects of the single protein product antithrombin III (ATIII) which is purified from fraction IV-1 paste on the myotube formation assay described in FIG. 45. All data were normalized to ATIII vehicle. ATIII displayed no significant activity.

### J. Example 10—C2C12 Myotube Metabolic Assay

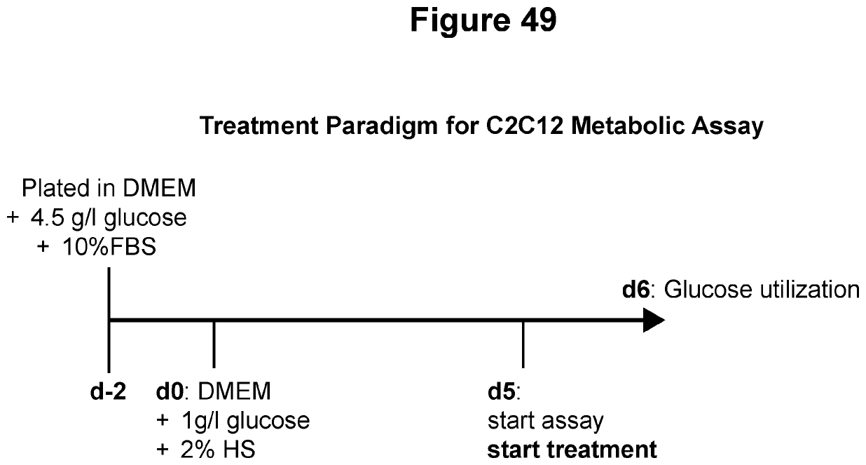

FIG. 49 is a depiction of the treatment paradigm for C2C12-derirved myotube formation. C2C12 myoblasts were seeded at 8 k/well in a 96 well plate in DMEM medium plus 4.5 g/L glucose and 10% fetal bovine serum (FBS). After 2 days they were differentiated to myotubes by changing the media to 1 g/L glucose and 2% horse serum. After 5 days, the cells were fully differentiated into myotubes and treated with various plasma fractions for 24 hours. The amounts of glucose used by the cells were determined by quantitative colorimetric assay. The amount of glucose utilized reflects the metabolic activity of the cells. Increase in metabolism of cells indicates a potential treatment of the following diseases, diabetes II, stroke, fatty liver, insulin resistance, certain metabolic myopathies and cardiovascular diseases.

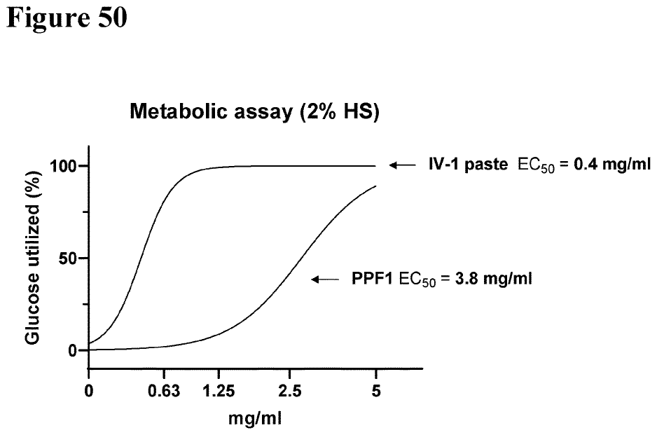

FIG. 50 shows the results of a dose-response relationship between PPF1, and fraction IV-1 paste in the glucose utilization assay described in FIG. 49. Glucose utilization was normalized and compared between treatments. Fraction IV-1 paste exhibits increased efficacy than PPF1 with an effective concentration 50% (EC₅₀) 9.5-fold more potent than PPF1.

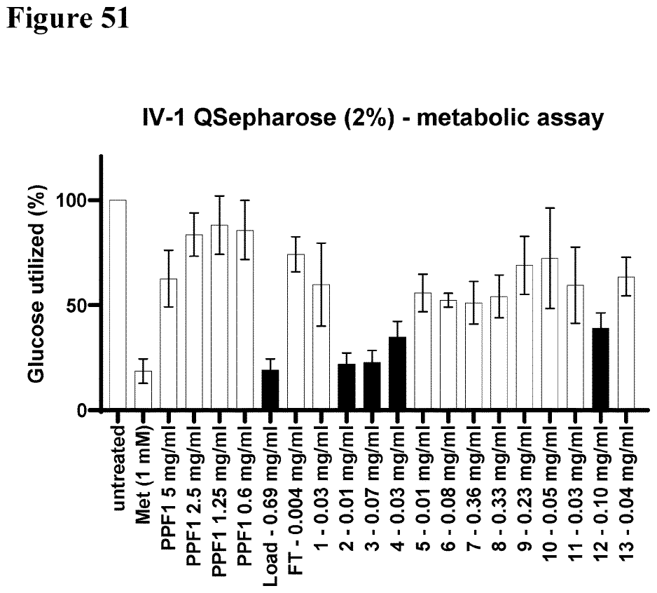

FIG. 51 reports the results of the metabolic assay described in FIG. 49 using the 13 subfractions from fraction IV-1 paste. Subfractions 2-4 and 12 demonstrated significant activity in glucose utilization. Again, it was unexpected that fractions 2-4 would be useful subfractions for muscle disease since it was unknown whether this subfractionation would separate the bioactives so precisely.

The results observed over this assortment of diverse assays were also surprising since they suggest that each assay can have its own set of bioactive drivers and the resolution in identifying these drivers was due to the quality of the subfractionation strategy. This allows for the development of multiple potential therapeutic fractions from IV-1 paste using this subfraction method. Additionally, the overall data showed that traditional protein products that can be derived from Fraction IV-1 paste (e.g., alpha-1 antitrypsin and antithrombin III) do not significantly contribute to the activities tested in these assays. Instead, it suggests that the subfractions have standalone factors that can be used to treat disease indications, and indeed the subfractions themselves may also be used as such.

### K. Example 10—Sub-Fractionation of Fraction IV-1 Suspension

Fraction IV-1 suspension was separated into distinct protein pools by Q Sepharose chromatography. The column was a 5.0×19.5 cm (442 ml) Q Sepharose Fast Flow (FF) run that was performed with 25 mmM Tris-HCl buffer at a pH of 8.0, run at 75 cm/h. The column was loaded with 450 ml of the Fraction IV-1 suspension, which corresponded to about 5 g of protein.

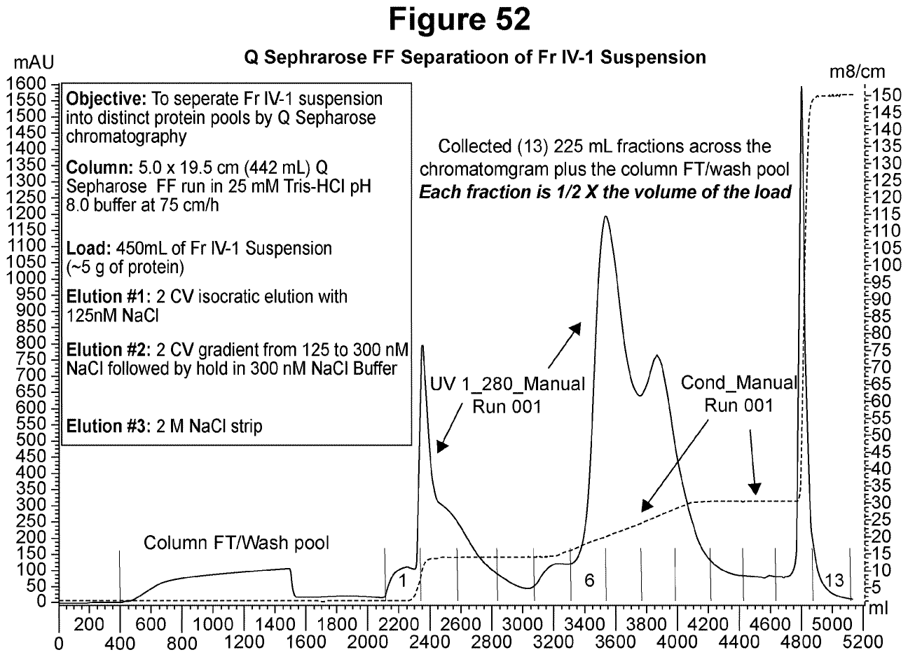

FIG. 52 shows the chromatographic results corresponding to the Q Sepharose separation of the Fraction IV-1 suspension. As shown by the light blue, vertical lines at the bottom of the figure, the column FT/wash pool was from 400 ml to 2100 ml. Next, the thirteen fractions of 225 ml each were collected (fractions 1, 6, and 13 are labeled for reference). Each fraction was half the volume of the load. The left vertical axis shows mAU and ranges from 0 to 1600. The left vertical axis corresponds to the signal at a UV wavelength of 280 nm. In contrast, the right vertical axis shows mS/cm and ranges from 0 to 150. Elution #1 was 2 CV isocratic elution with 125 mM NaCl, whereas Elution #2 was 2 CV gradient from 125 to 300 mM NaCl, followed by hold in 300 mM NaCl buffer. Elution #3 was 2 M NaCl strip.

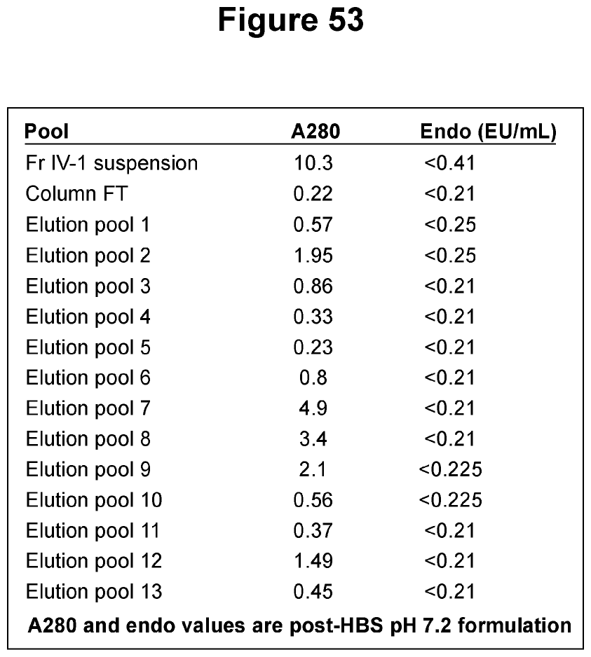

FIG. 53 shows the elution pools observed during the experiment. The A280 and endo values were post-HBS pH 7.2 formulation. The largest values were observed with elution pools 2, 7, 8, 9, and 12, which corresponded to the peaks in the mAU signal, as shown in FIG. 52.

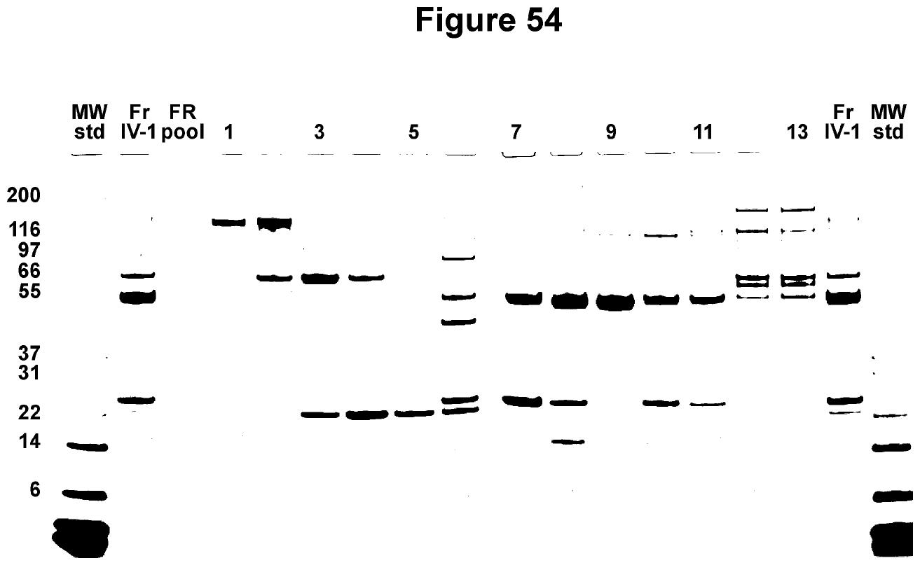

FIG. 54 shows the results of gel electrophoresis performed on the elution pools that were obtained from the Fraction IV-1 suspension separation by Q Sepharose chromatography. The left column was the MW standard, which was followed by the Fraction IV-1 and FT pool samples. Additionally, elution pools 1 through 13 were recorded, followed by another Fraction IV-1 and MW standard. Significant signals were found at: 116 in pools 1 and 2, 66 in pools 2-4, 55 in pools 6-13, and 22 in pools 3-6. Other signals were also found.

### L. Example 11—SH-SY5Y Survival Assay

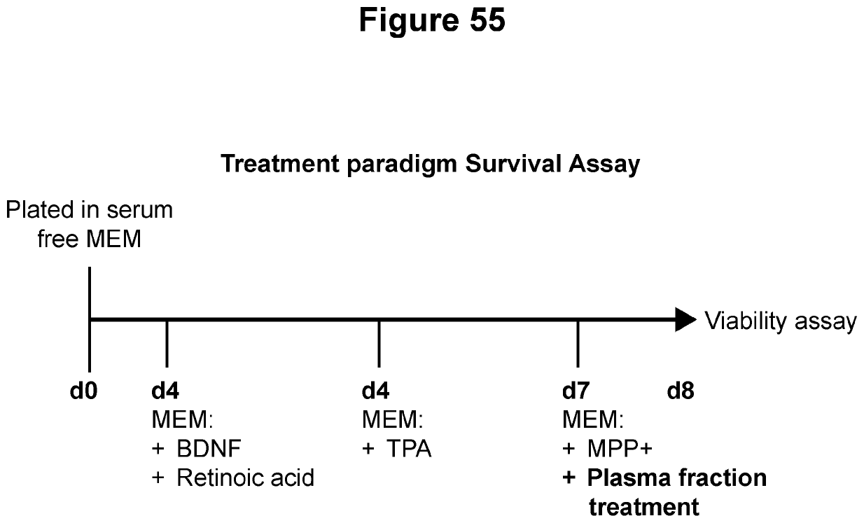

FIG. 55 SH-SY5Y cells were seeded at 20 k/well in a 96 well plate in MEM medium without serum. At day 1 the differentiation of the cells to dopaminergic neurons was initiated by the addition of BDNF (50 ng/ml) and Retinoic acid (5 μM). After 4 days the complete media was changed and TPA (80 nM) was added to the MEM media without serum. On day 7, after cells had differentiated to dopaminergic neurons, cells were stressed with a neurotoxin (MPP+1 mM) for 24 hours while treated concurrently with plasma fractions. The number of cells surviving the neurotoxin stress were quantified at day 8. The viability of cells was quantified using a fluorescence dye (Promega kit). The amount of fluorescence reflects the viability of cells. Increase in dopaminergic neuronal cell survival indicates a potential treatment of neurodegenerative diseases, such as PD.

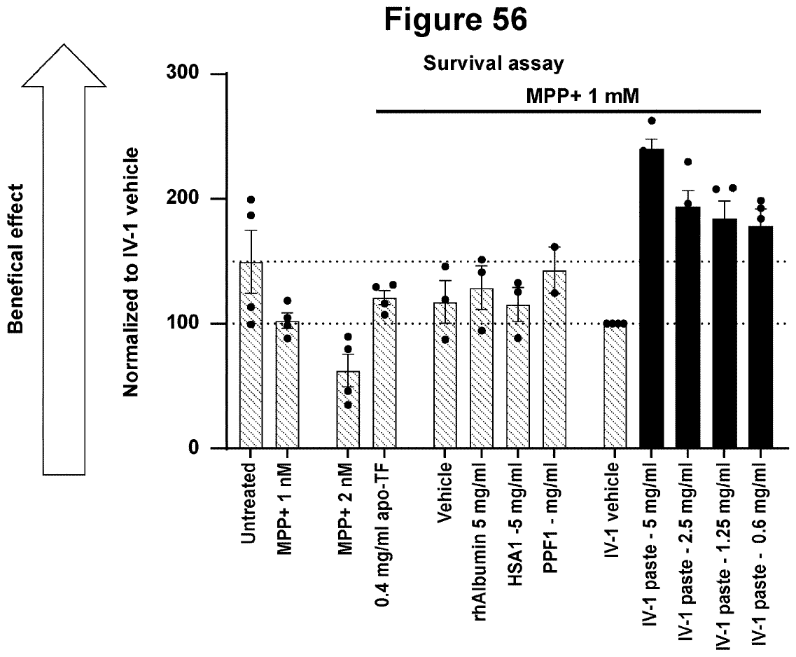

FIG. 56 shows the results of the survival assay performed on dopaminergic, under neurotoxin stress (MPP+1 mM), using multiple plasma fractions (HAS1, PPF1, and IV-1 paste) or recombinant human albumin (rhAlbumin). The plasma fractions and rhAlbumin were administered as described in FIG. 55. IV-1 paste was tested in a dose responsive manner in a range from 0.6 mg/mL-5 mg/mL. A negative control (2 mM MPP+) and positive control (0.4 mg/mL Apo-Transferrin) were both administered to cells also in the same manner as the plasma fractions described in FIG. 55. FIG. 56 shows that dopaminergic neurons stressed with a neurotoxin were rescued by IV-1 paste treatment in a dose dependent manner. This indicates that in a disease context where dopaminergic neurons are dying, the death of the cell may be attenuated. The findings are unexpected and surprising since the beneficial activity of IV-1 paste exceeded the beneficial activity of the positive control and of the other Plasma Fraction several times.

### M. Example 12—SH-SY5Y Reactive Oxygen Species (ROS) Assay

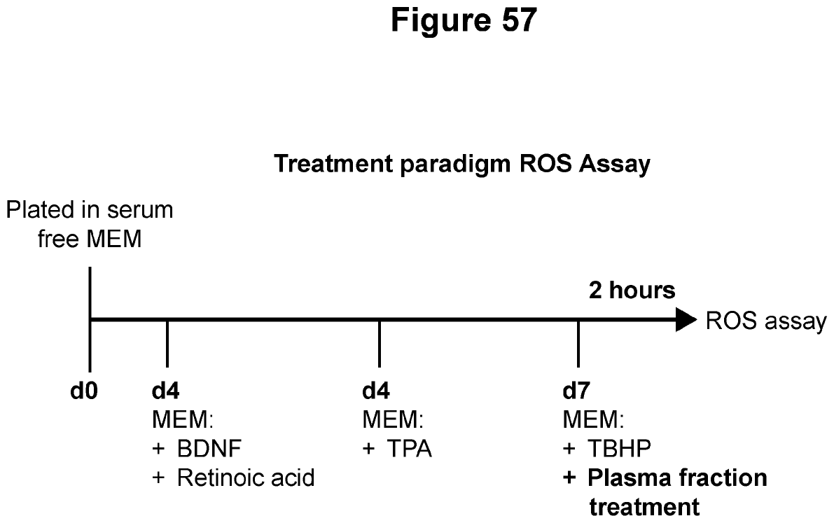

FIG. 57 SH-SY5Y cells were seeded at 20 k/well in a 96 well plate in MEM medium without serum. At day 1 the differentiation of the cells to dopaminergic neurons was initiated by the addition of BDNF (50 ng/ml) and Retinoic acid (5 μM). After 4 days the complete media was changed and TPA (80 nM) was added to the MEM media without serum. On day 7, after cells had differentiated to dopaminergic neurons, cells were stressed with a hydroperoxide (TBHP 50 μM) to increase ROS production while treated concurrently with plasma fractions. The increase in ROS production was quantified after 2 hours using a fluorescence dye (Abcam kit). The amount of fluorescence reflects the amount of ROS produced. Decrease in ROS production in dopaminergic neurons indicates a potential treatment of neurodegenerative diseases, such as PD.

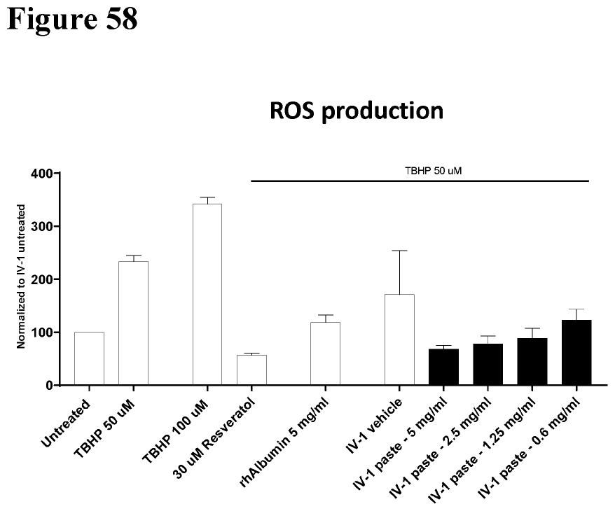

FIG. 58 shows the results of the ROS production assay on dopaminergic neurons, under hyperoxide stress (TBHP 50 μM), using a plasma fraction (IV-1 paste) or recombinant human albumin (rhAlbumin). The IV-1 paste and rhAlbumin were administered as described in FIG. 57. IV-1 paste was tested in a dose responsive manner in a range from 0.6 mg/mL-5 mg/mL. A negative control (100 μM TBHP) and positive control (30 μM Resveratrol) were both administered to cells also in the same manner as the plasma fraction described in FIG. 57. FIG. 58 shows that dopaminergic neurons stressed with peroxidase produced less ROS if they were treated with IV-1 paste in a dose dependent manner. This suggests that in a disease context where dopaminergic neurons have an increased ROS production, the detrimental effect of ROS may be attenuated.

### N. Example 13—SH-SY5Y Survival Assay treatment with purified protein products (A1AT and ATIII)

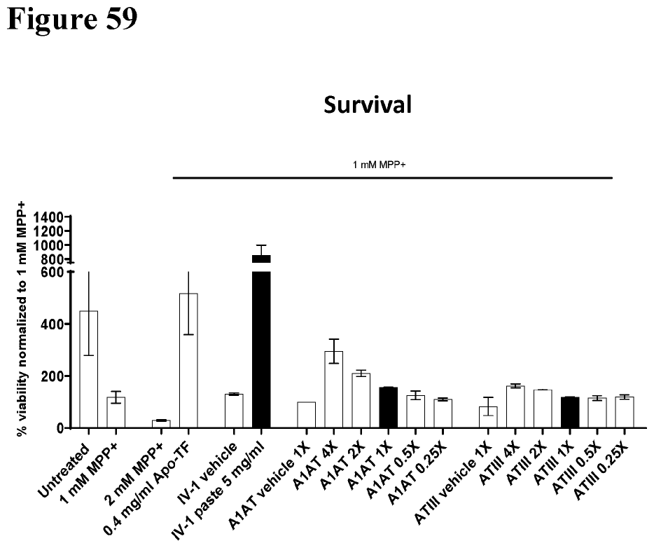

FIG. 59 shows the results of the survival assay performed on dopaminergic neurons, under neurotoxin stress (MPP+1 mM), using single purified proteins (A1AT and ATIII). A1AT and ATIII were administered as described in FIG. 55. IV-1 paste was tested at 5 mg/mL and showed significant activity. A negative control (2 mM MPP+) and positive control (0.4 mg/mL Apo-Transferrin) were both administered to cells also in the same manner as the plasma fraction described in FIG. 55. A1AT demonstrated slight but not significant activity in the survival assay. Additionally, a dose response was performed for A1AT and ATIII. The black bar represents the same amount of protein as is in parent IV-1 fraction (i.e., 1×). ATIII did not significantly impact the survival of SH-SY5Y cells, but A1AT did demonstrate activity in a dose responsive manner. Surprisingly, as the amount present in IV-1 paste, A1AT only had a slight effect compared to IV-1 paste itself, suggesting that there are additional bioactivities in IV-1 paste not accounted for by A1AT.

### O. Example 14—SH-SY5Y Reactive Oxygen Species (ROS) Assay Treatment with Purified Protein Products (A1AT and ATIII)

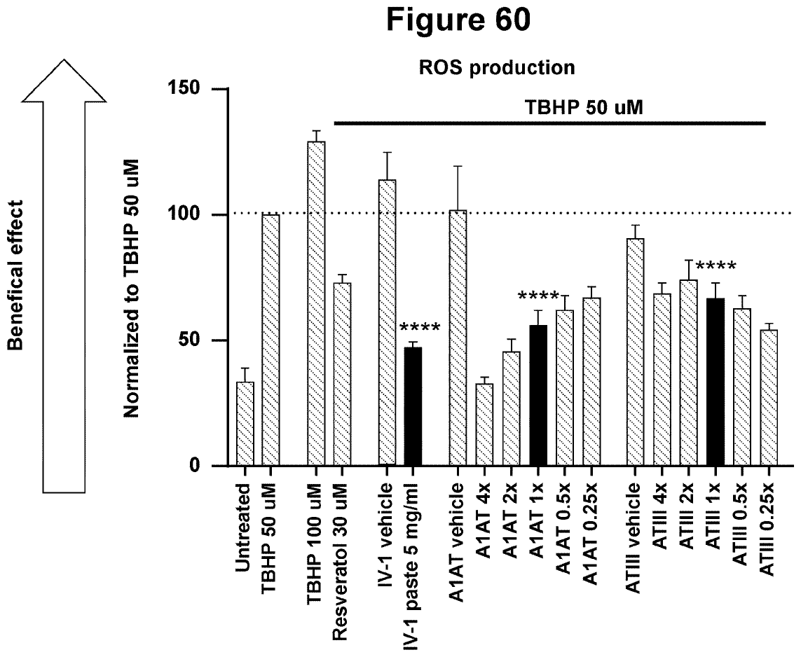

FIG. 60 shows the results of the ROS production assay on dopaminergic neurons, under hyperoxide stress (TBHP 50 μM), single purified proteins (A1AT and ATIII). A1AT and ATIII were administered as described in FIG. 57. IV-1 paste was tested at 5 mg/mL and showed significant activity. A negative control (100 μM TBHP) and positive control (30 μM Resveratrol) were both administered to cells also in the same manner as the plasma fraction described in FIG. 57. FIG. 60 shows that dopaminergic neurons stressed with peroxidase produced less ROS if they were treated with IV-1 paste. Also, A1AT and ATIII demonstrated significant activity in the survival assay. Additionally, a dose response was performed for A1AT and ATIII. The black bar represents the same amount of protein as is in parent IV-1 fraction (i.e., 1×). A1AT, but not ATIII, did demonstrate activity in a dose responsive manner. Surprisingly, as the amount present in IV-1 paste, A1AT and ATIII did significantly reduce ROS production in dopaminergic neurons stressed with peroxidase, suggesting that A1AT and ATIII are bioactivities in IV-1 paste. This furthermore suggests that in a disease context where dopaminergic neurons have an increased ROS production, the detrimental effect of ROS may be attenuated by either IV-1 paste, A1AT and ATIII treatment.

### P. Example 15—SureQuant for Most Abundant Proteins in IV-1 Paste Compared to PPF1

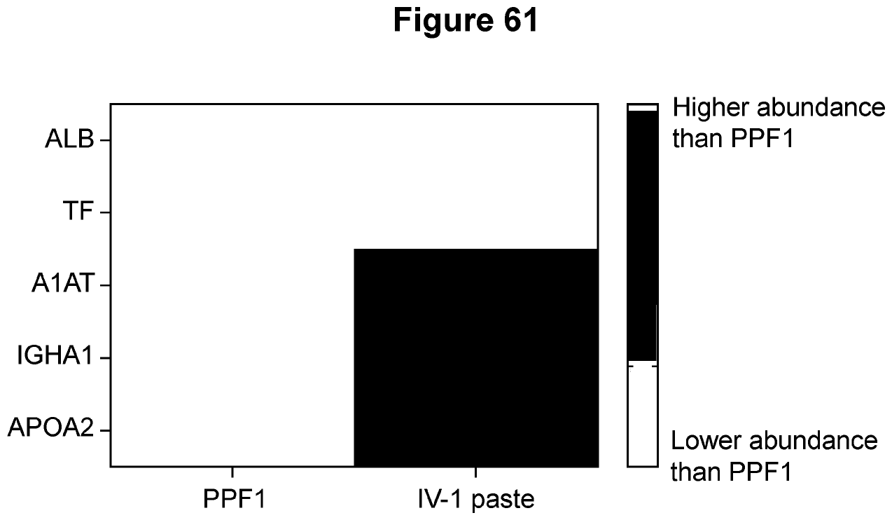

FIG. 61 shows the heatmap for abundant proteins in fraction IV-1 paste, normalized to amount of protein in fraction PPF1. Protein abundance was quantified by a quantitative mass spectrometry-based method known as SureQuant. Compared to PPF1, IV-1 paste is enriched for several beneficial proteins including A1AT, TF, IGHA1 and APOA2. This suggests that these proteins might contribute to the beneficial effects we have outlined in FIG. 1-60.

### Q. Example 16—SureQuant for IGF and related proteins in IV-1 paste compared to PPF1

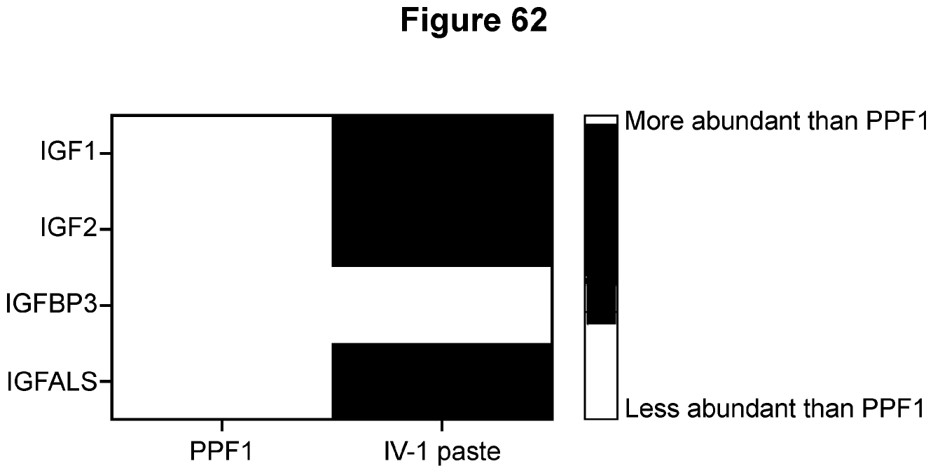

FIG. 62 shows the heatmap for IGF1, IGF2, IGFBP3 and IGFALS proteins in fraction IV-1 paste, normalized to amount of protein in fraction PPF1. Protein abundance was quantified by a quantitative mass spectrometry-based method known as SureQuant. The figure compares the abundancy of IGF1, IGF2 and the most important binding proteins (IGFBP3 and IGFALS) in the IGF signaling pathway. Compared to PPF1, IV-1 paste is enriched for both IGF1 and IGF2 and is enriched for the binding proteins (IGFBP3 and IGFALS). This suggests that these proteins might contribute to the beneficial effects we have outlined in FIGS. 1-60.

### R. Example 17—Transcriptomic Analysis: IV-1 Paste Treated Cells Showed High Differential Gene Expression Across Two Muscle Assays

IV-1 paste shows the strongest effect compared to other plasma fractions, including PPF1, in differential gene expression across two muscle assays. Plasma Fractions were administrated to C2C12 cells as described in FIGS. 45 and 49. Transcriptomic analysis showed that fraction IV-1 paste significantly changed gene expression in the following pathways: glucose metabolism, glycolysis, gluconeogenesis, glycogen metabolism, oxidative phosphorylation, extracellular matrix reorganization, collagen degradation and formation, muscle contraction, cell cycle, proliferation, ion homeostasis, neutrophil degranulation, inflammation. This suggests that the proteins enriched in fraction IV-1 paste modulate multiple pathways and account for the beneficial effects outlined in FIGS. 1-60. In conclusion the transcriptomic data supports our theory that fraction IV-1 paste is geared towards multimodal activity and poly-pharmacology.

1. A method of treating a disease in a subject diagnosed with the disease, the method comprising administering an effective amount of a Fraction IV-1 subfraction to the subject.

2. The method of claim 1 wherein the Fraction IV-1 subfraction is one of the thirteen subfractions obtained by the process described in Example 10.

3. The method of claim 1 wherein the Fraction IV-1 subfraction is at least one of Fraction IV-1 subfraction 2, 3, 4, and 12.

4. The method of claim 3 wherein the disease is one or more of the following: diabetes type II, stroke, fatty liver disease, insulin resistance, metabolic myopathy, and cardiovascular disease.

5. The method of claim 1 wherein the Fraction IV-1 subfraction is at least one of Fraction IV-1 subfraction 7 and 8.

6. The method of claim 5 wherein the disease is a neurodegenerative disease.

7. The method of claim 1 wherein the Fraction IV-1 subfraction is at least one of Fraction IV-1 subfraction 2, 3, 4, 5, 6, and 12.

8. The method of claim 7 wherein the disease is one or more of the following: Cachexia, frailty, Charcot-Marie-tooth disease, muscular dystrophy, congenital fiber type disproportion.

9. A composition comprising a Fraction IV-1 paste subfraction obtainable by the process described in Example 10.

10. The Fraction IV-1 paste subfraction of claim 9 wherein the subfraction is selected from the following: subfraction 1, subfraction 2, subfraction 3, subfraction 4, subfraction 5, subfraction 6, subfraction 7, subfraction 8, subfraction 9, subfraction 10, subfraction 11, subfraction 12, and subfraction 13.

11. The method of claim 5 wherein the neurodegenerative disease is Parkinson's disease.

12. The method of claim 11 wherein the Parkinson's disease is associated with degenerating dopaminergic neurons having increased reactive oxygen species production.
